(19) United States
(12) Patent Application Publication
(10) Pub. No.: US 2003/0176477 A1
(43) Pub. Date: Sep. 18, 2003

(54) Isothiazolecarboxylic acid derivatives and their use as microbicides

(76) Inventors: Yoshinori Kitagawa, Tochigi (JP); Koichi Ishikawa, Tochigi (JP); Haruko Sawada, Ibaraki (JP); Yasuo Araki, Tochigi (JP); Lutz Assmann, Langenfeld (DE)

Correspondence Address: BAYER CORPORATION, PATENT DEPARTMENT, 100 BAYER ROAD, PITTSBURGH, PA, 15205, US

(21) Appl. No.: 10/182,248

(22) PCT Filed: Jan. 23, 2001
(86) PCT No.: PCT/EP01/00682

(30) Foreign Application Priority Data
Jan. 28, 2000 (JP) 2000-19920

(51) Int. Cl. A61K 31/425; C07D 275/02

(52) U.S. Cl. 514/372; 548/214

(57) ABSTRACT

Novel isothiazolecarboxylic acid derivatives of the formula (I), in which A, Q, Z and k have the meanings mentioned in the specification, processes for the preparation of the new compounds and their use as microbicides.

The present invention relates to novel isothiazolecarboxylic acid derivatives, to processes for their preparation and to their use as microbicides.

It has already been known that certain isothiazolecarboxylic acid derivatives can be employed for the control of plant pests (see JP-A 59 024/1993, DE-A 1 97 50 011 and DE-A 197 50 012). The fungicidal activity of such known compounds, however, is not always satisfactory.

There have now been found novel isothiazolecarboxylic acid derivatives of the formula

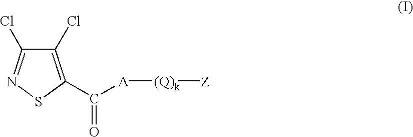

wherein

A represents an oxygen atom, a sulphur atom or a group of the formula

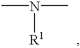

in which

R¹ represents a hydrogen atom, C₁₋₄ alkyl, C₃₋₆ cycloalkyl, phenyl or 2-hydroxyethyl,

Q represents a group selected from

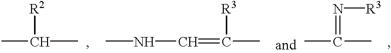

in which

R² represents a hydrogen atom, C₁₋₄ alkyl, C₁₋₄ haloalkyl, C₇₋₉ aralkyl or phenoxymethyl, which may be substituted by C₁₋₄ alkoxy-carbonyl, and

R³ represents phenyl, optionally substituted by halogen, C₁₋₄ alkyl, C₁₋₄ haloalkyl, C₁₋₄ alkoxy, C₁₋₄ haloalkyl, phenoxy, benzyloxy, cyano, oxydimethylene and/or nitro,

or represents naphthyl,

k represents 0 or 1, and

Z represents a 5-7-membered heterocyclic ring comprising 1 to 4 nitrogen atoms, wherein the heterocyclic ring may be substituted by one or more substituents selected from halogen, C₁₋₄ alkyl, C₁₋₄ alkoxy, C₁₋₄ haloalkoxy, C₃₋₆ cycloalkoxy, C₂₋₄ alkenyl, phenyl, halophenyl, oxo and/or spiro-bonded C₃₋₆ alicyclic groups, and wherein the heterocyclic ring may be condensed with a benzene or cyclohexene ring, or

Z represents a 5-7-membered heterocyclic ring comprising at least one nitrogen atom and one oxygen atom, or comprising at least one nitrogen atom and one sulphur atom, wherein each of the heterocyclic rings may be substituted by one or more substituents selected from C₁₋₄ alkyl, phenyl and/or oxo, or

Z represents cyano or a group selected from

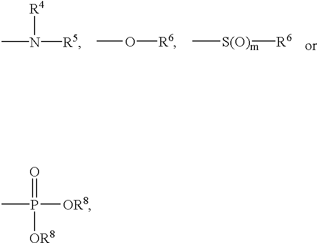

in which

R⁴ represents a hydrogen atom, C₁₋₄ alkyl, benzyl or phenyl, the last two radicals being optionally substituted by one to three radicals selected from halogen and/or C₁₋₄ alkyl, or

R⁴ represents tetrazol-5-yl-thiomethyl, which may be substituted by C₁₋₄ alkyl,

R⁵ represents formyl, C₁₋₄ alkylcarbonyl, 3-4-dichloroisothiazol-5-yl-carbonyl, C₁₋₄ alkylsulphonyl or phenylsulphonyl or

R⁵ represents phenylcarbonyl, optionally substituted by one to three radicals selected from halogen and C₁₋₄ alkyl,

R⁶ represents a hydrogen atom, C₁₋₄ alkyl, C₁₋₄ haloalkyl, benzyl, halogen-substituted benzyl, phenyl, halogen-substituted phenyl, C₁₋₄ alkylcarbonyl, benzoyl, C₁₋₄ haloalkyl-substituted benzoyl, phenylcarbamoyl or C₁₋₄ haloalkyl-substituted phenylcarbamoyl,

R⁷ represents C₁₋₄ alkyl, benzyl or phenyl the last two radicals being optionally substituted by one to three radicals selected from C₁₋₄ alkyl and/or halogen, or

R⁷ represents tetrazol-5-yl or

R⁷ represents thiadiazol-2-yl optionally substituted by C₁₋₄ alkyl or phenyl, or

R⁷ represents 2-thiazoline-2-yl, C₁₋₄ alkylcarbonyl or benzoyl,

m represents 0, 1 or 2, and

R⁸ represents C₁₋₄ alkyl,

or, in case

A represents a

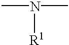

group, then

R¹, Q and Z may represent a 5- or 6-membered heterocyclic group comprising 1-3 nitrogen atoms and being optionally substituted by one to three radicals selected from C₁₋₄ alkyl, C₁₋₄ haloalkyl, hydroxy, oxo, hydroxymethyl or phenyl, which in turn may be substituted by halogen and/or C₁₋₄ alkyl, or

-(Q)ₖ-Z represents a group selected from

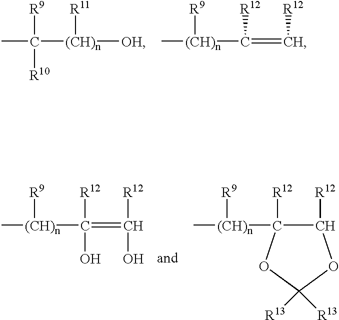

wherein

n represents 1 or 2,

R⁹ represents a hydrogen atom or C₁₋₄ alkyl,

R¹⁰ represents a hydrogen atom, hydroxymethyl or benzyl which may be substituted by 1 to 3 halogen atoms,

R¹¹ represents a hydrogen atom, C₁₋₄ alkyl or phenyl,

R¹² represents a hydrogen atom, C₁₋₄ alkyl or phenyl, or two of the R¹² radicals, together with the carbon atoms to which they are bonded, may form a 5- or 6-membered hydrocarbon ring, and

R¹³ represents a hydrogen atom, C₁₋₉ alkyl, C₃₋₆ cycloalkyl, C₇₋₈ arylalkyl, C₃₋₆ cycloalkyl-C₁₋₄ alkyl, C₁₋₄ alkoxy-C₁₋₄ alkyl or di-(C₁₋₄ alkoxy)-methyl, or the two R¹³ radicals, together with the carbon atom to which they are bonded, form a C₅₋₆ alicyclic ring which is optionally substituted by C₁₋₄ alkyl, or

-A-(Q)ₖ-Z represent —SH or a group of the formula

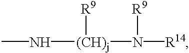

in which

R⁹ has the above-mentioned meanings,

R¹⁴ represents C₁₋₄ alkyl, C₃₋₆ cycloalkyl or hydroxy-substituted C₂₋₄ alkyl, and

j represents 2, 3 or 4,

or, in case

A represents

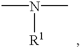

Q represents

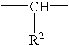

and

Z represents

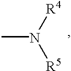

these

radicals together may represent a group of the formula

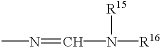

in which

R¹⁵ and R¹⁶ independently of one another represent C₁₋₄ alkyl or phenyl or

R¹⁵ and R¹⁶ together with the nitrogen atom, to which they are bonded, form a 5- or 6-membered heterocyclic group comprising at least one nitrogen atom or comprising at least one nitrogen atom and one oxygen atom,

with the proviso that

in case

Q represents a group of the formula

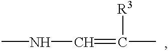

then

A represents

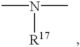

wherein

R¹⁷ represents a hydrogen atom or C₁₋₄ alkyl, and

Z represents cyano,

and in case

Q represents a group of the formula

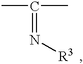

then

A represents —NH and

Z represents cyano

and in case

-(Q)ₖ-Z represents 2,3-dihydroxypropyl, then

A represents a sulphur atom or a group of the formula

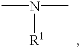

and in case

-(Q)ₖ-Z represents 2-hydroxyethyl and

A represents a group of the formula

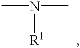

then

R¹ represents C₁₋₄ alkyl, C₃₋₆ cycloalkyl, phenyl or 2-hydroxyethyl,

and in case

A represents a group of the formula

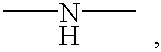

then

Q represents —CH₂— and

Z represents a group of the formula

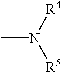

in which

R⁴ represents a hydrogen atom, benzyl or phenyl, the last two radicals being optionally substituted by halogen and/or C₁₋₄ alkyl, and

R⁵ represents formyl,

and with the further proviso that

Z does not represent cyano or a group selected from

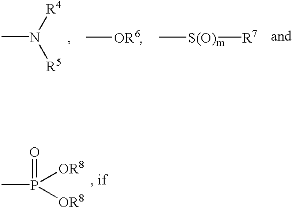

A is oxygen or sulphur and

k is o.

Further, it has been found that isothiazolecarboxylic acid derivatives of the formula (I) can be prepared by several processes. Thus,

a) the compound of the formula (I), in which

-A-(Q)ₖ-Z represents a group of the formula

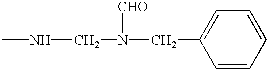

can be prepared by reacting 3,4-dichloro-isothiazole-5-carboxamide of the formula

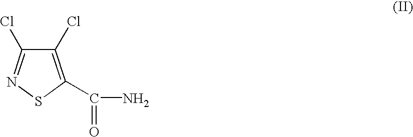

with the formylamine of the formula

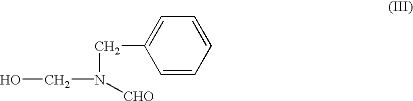

in the presence of an inert diluent and, if appropriate, in the presence of a catalyst, or

b) compounds of the formula (I), in which

-A-(Q)ₖ-Z represents a group of the formula

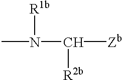

in which

R^{1b} represents a hydrogen atom or C₁₋₄ alkyl,

R^{2b} represents a hydrogen atom or C₁₋₄ haloalkyl and

Z^{b} represents a group selected from

—OR⁶, —SR⁷, —SO₂—R⁷ and

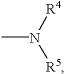

in which

R⁴, R⁵, R⁶ and R⁷ have the above-mentioned meanings,

can be prepared by reacting isothiazole derivatives of the formula

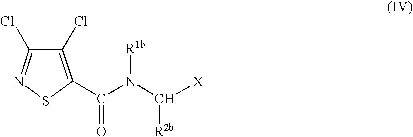

in which

R^{1b} and R^{2b} have the above-mentioned meanings and

X is chloro or bromo,

with compounds of the formula

M-Z^{b} (V)

in which

Z^{b} has the above-mentioned meanings and

M represents a hydrogen atom, lithium, sodium or potassium,

in the presence of an inert diluent and, if appropriate, in the presence of an acid binding agent and, if appropriate, in the presence of a phase-transfer catalyst, or

c) compounds of the formula (I), in which

-A-(Q)ₖ-Z represents a group of the formula

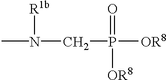

in which

R^{1b} and R⁸ have the above-mentioned meanings,

can be prepared by reacting isothiazole derivatives of the formula

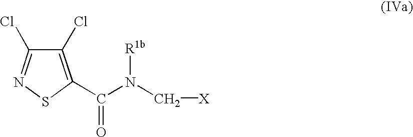

in which

R^{1b} and X have the above-mentioned meanings,

with phosphorous compounds of the formula

P(OR⁸)₃ (VI)

in which

R⁸ has the above-mentioned meanings,

in the presence of an inert diluent, or

d) compounds of the formula (I), in which

-A(Q)ₖ-Z represents a group of the formula

-A^{d}-CH₂-Z^{d},

in which

A^{d} represents

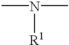

or a sulphur atom, wherein

R¹ has the above-mentioned meanings, and

Z^{d} represents a 5-7-membered heterocyclic ring comprising 1 to 4 nitrogen atoms, wherein the heterocyclic ring may be substituted by one or more substituents selected from halogen, C₁₋₄ alkyl, C₁₋₄ alkoxy, C₁₋₄ haloalkyl, C₃₋₆ cycloalkyl, C₂₋₄ alkenyl, phenyl or

Z^{d} represents a 5-7-membered heterocyclic ring comprising at least one nitrogen atom and one oxygen atom, or comprising at least one nitrogen atom and one sulphur atom, wherein each of the heterocyclic rings may be substituted by one or more substituents selected from C₁₋₄ alkyl, phenyl and/or oxo or

Z^{d} represents a group selected from

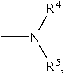

—OR⁶ a —SR⁷

in which

R⁴, R⁵, R⁶ and R⁷ have the above-mentioned meanings,

can be prepared by reacting isothiazole derivatives of the formula

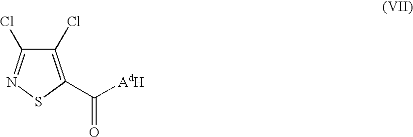

in which

A^{d} has the above-mentioned meaning,

with chloromethyl compounds of the formula

Cl—CH₂-Z^{d} (VIII)

in which

Z^{d} has the above-mentioned meanings,

in the presence of an inert diluent and, if appropriate, in the presence of an acid binding agent, or

e) compounds of the formula (I), in which

-A-(Q)ₖ-Z represents a group of the formula

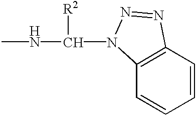

in which

R² has the above-mentioned meanings,

can be prepared by reacting 3,4-dichloro-isothiazole-5-carboxamide of the formula

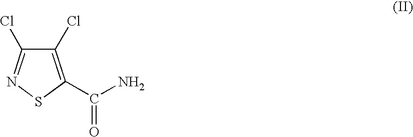

with formyl compounds of the formula

R²—CHO (IX)

in which

R² has the above-mentioned meanings,

and with 1H-benzotriazole of the formula

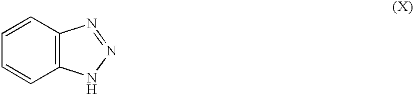

in the presence of an inert diluent and, if appropriate, in the presence of a catalyst, or

f) compounds of the formula (I), in which

-A-(Q)ₖ-Z represents —SH or a group selected from

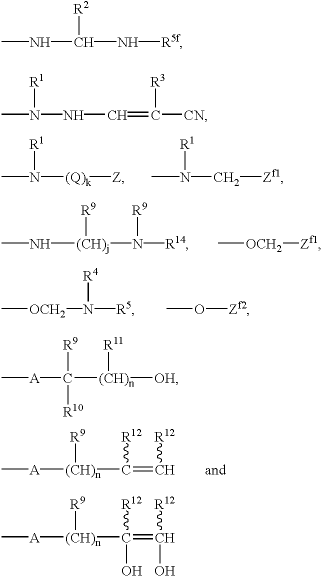

in which

A, Q, Z, j, k, n, R¹, R², R³, R⁴, R⁵, R⁹, R¹⁰, R¹¹, R¹² and R¹⁴ have the above-mentioned meanings,

Z^{f1} represents a 5-7-membered heterocyclic ring comprising 1 to 4 nitrogen atoms, wherein the heterocyclic ring may be substituted by one or more substituents selected from halogen, CIA alkyl, C₁₋₄ alkoxy, C₁₋₄ haloalkyl, C₃₋₆ cycloalkyl, C₂₋₄ alkenyl, phenyl, halophenyl, oxo and/or spiro-bonded C₃₋₆ alicyclic groups, and wherein the heterocyclic ring may be condensed with a benzene or cyclohexene ring, or

Z^{f1} represents a 5-7-membered heterocyclic ring comprising at least one nitrogen atom and one oxygen, or comprising at least one nitrogen atom and one sulphur atom, wherein each of the heterocyclic rings may be substituted by one or more substituents selected from C₁₋₄ alkyl, phenyl and/or oxo,

Z^{f2} represents a 5-membered heterocyclic group comprising 1 or 2 nitrogen atoms, which heterocycle may be substituted by C₁₋₄ alkyl and/or oxo, and

R^{5r} represents formyl, C₁₋₄ alkylcarbonyl or phenylcarbonyl, this latter radical being optionally substituted by 1 to 3 radicals selected from halogen and C₁₋₄ alkyl,

can be prepared by reacting 3,4-dichloro-isothiazole-5-carbonyl chloride of the formula

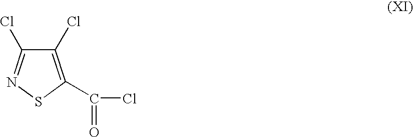

with compounds of the formula

M-Y¹ (XII)

in which

M has the above-mentioned meanings and

Y¹ represents —SH or a group selected from

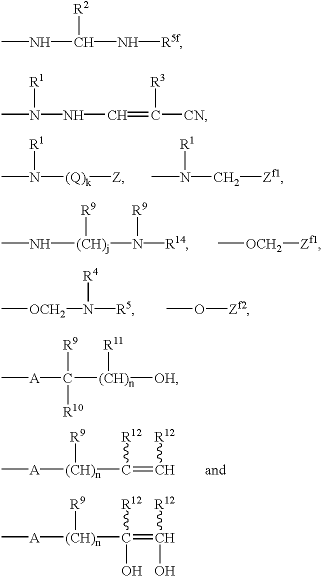

in which

A, Q, Z, j, k, n, R¹, R², R³, R⁴, R⁵, R⁹, R¹⁰, R¹¹, R¹², R¹⁴, Z^{f1}, Z^{f2}, and R^{5f} have the above-mentioned meanings,

in the presence of an inert diluent and, if appropriate, in the presence of an acid binding agent, or

g) compounds of the formula (I), in which

-A-(Q)ₖ-Z represents a group selected from

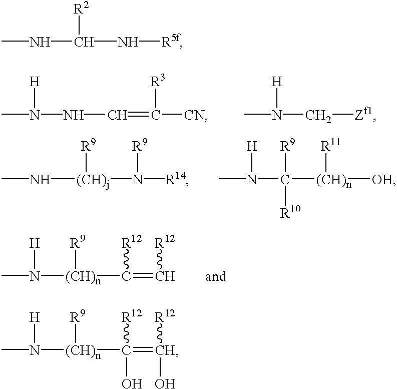

in which

Z^{f1}, j, n, R², R³, R⁹, R¹⁰, R¹¹, R¹², R¹⁴ and R^{5f} have the above-mentioned meanings,

can be prepared by reacting 3,4-dichloro-isothiazole-5-carboxylic acid esters of the formula

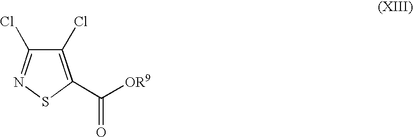

in which

R^{g} represents C₁₋₄ alkyl

with compounds of the formula

H—Y² (XIV)

in which

Y² represents a group selected from

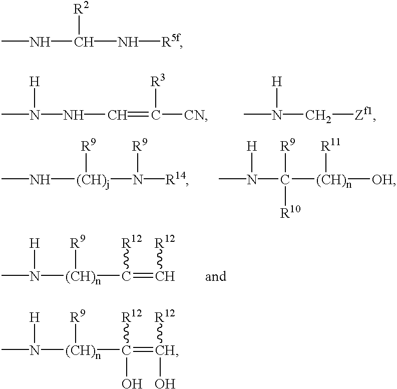

in which

Z^{f1}, j, n, R², R³, R⁹, R¹⁰, R¹¹, R¹², R¹⁴, and R^{5f} have the above-mentioned meanings,

in the presence of an inert diluent and, if appropriate, in the presence of an acid binding agent, or

h) compounds of the formula (I), in which

-A-(Q)ₖ-Z represents a group of the formula

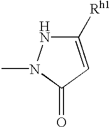

in which

R^{h1} represents phenyl optionally substituted by halogen and/or C₁₋₄ alkyl,

can be prepared by reacting 3,4-dichloro-isothiazole-5-carbohydrazide of the formula

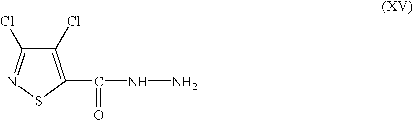

with compounds of the formula

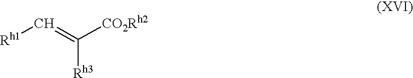

in which

R^{h1} has the above-mentioned meanings,

R^{h2} represents C₁₋₄ alkyl and

R^{h3} represents cyano or —COOR^{h2},

in the presence of an inert diluent and, if appropriate, in the presence of an acid binding agent and, if appropriate, in the presence of a catalyst, or

i) compounds of the formula (I), in which

-A-(Q)ₖ-Z represents a group of the formula

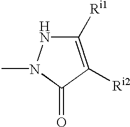

in which

R¹¹ represents a hydrogen atom or C₁₋₄ alkyl or represents phenyl $ optionally substituted by halogen and/or C₁₋₄ alkyl and

R¹² represents a hydrogen atom or C₁₋₄ alkyl,

can be prepared by reacting 3,4-dichloro-isothiazole-5-carbohydrazide of the formula

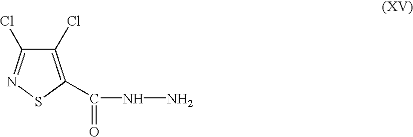

with compounds of the formula

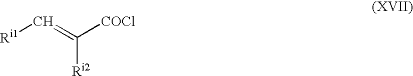

in which

R¹¹ and R¹² have the above-mentioned meanings,

in the presence of an inert diluent and, it appropriate, in the presence of an acid binding agent, or

j) compounds of the formula (I), in which

-A-(Q)ₖ-Z represents a group of the formula

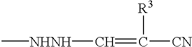

in which

R³ has the above-mentioned meanings,

can be prepared by reacting 3,4-dichloro-isothiazole-5-carbohydrazide of the formula

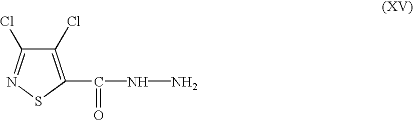

with compounds of the formula

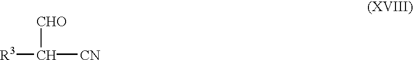

in which

R³ has the above-mentioned meanings,

in the presence of an inert diluent and, if appropriate, in the presence of a catalyst, or

k) compounds of the formula (I), in which

-A-(Q)ₖ-Z represents a group of the formula

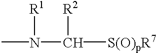

in which

R¹, R² and R⁷ have the above-mentioned meanings and

p denotes 1 or 2,

can be prepared by reacting isothiazolecarboxylic acid derivatives of the formula

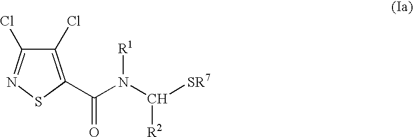

in which

R¹, R² and R⁷ have the above-mentioned meanings,

with oxidizing agents, which are suitable for providing oxygen, in the presence of an inert diluent, or

l) compounds of the formula (I), in which

-A-(Q)ₖ-Z represents a group of the formula

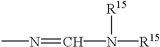

in which

R¹⁵ has the above-mentioned meanings,

can be prepared by reacting 3,4-dichloro-isothiazole-5-carboxamide of the formula

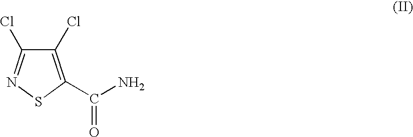

with compounds of the formula

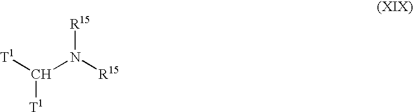

in which

R¹⁵ has the above-mentioned meanings and

T¹ represents C₁₋₄ alkoxy,

in the presence of an inert diluent and, if appropriate, in the presence of a catalyst, or

m) compounds of the formula (I), in which

-(Q)ₖ-Z represents a group of the formula

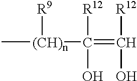

in which

R⁹, R¹² and n have the above-mentioned meanings,

can be prepared by reacting isothiazolecarboxylic acid derivatives of the formula

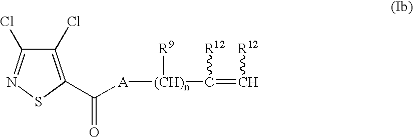

in which

R⁹, R¹² and n have the above-mentioned meanings,

with oxidizing agents, which are suitable for providing oxygen, in the presence of water and, if appropriate, in the presence of an inert organic diluent, or

n) compounds of the formula (I), in which

-(Q)ₖ-Z represents a group of the formula

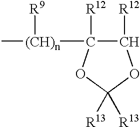

in which

R⁹, R¹², R¹³ and n have the above-mentioned meanings,

can be prepared by reacting isothiazolecarboxylic acid derivatives of the formula

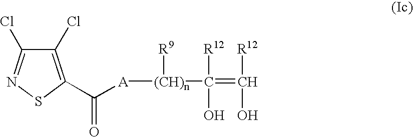

in which

A, n, R⁹ and R¹² have the above-mentioned meanings,

with carbonyl derivatives of formula

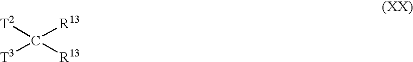

in which

R¹³ has the above-mentioned meanings and

T² represents C₁₋₄ alkoxy or the two T²-radicals together represent an oxo group,

in the presence of an inert diluent and, if appropriate, in the presence of an acid catalyst, or

o) compounds of the formula (I), in which

-A-(Q)ₖ-Z represents a group of the formula

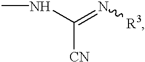

in which

R³ has the above-mentioned meanings,

can be prepared by reacting 3,4-dichloro-isothiazole-5-carboxamide of the formula

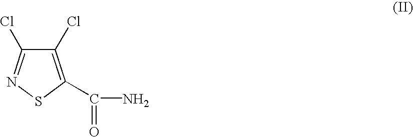

with cyano compounds of the formula

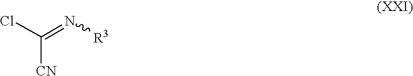

in which

R³ has the above-mentioned meanings,

in the presence of an inert diluent and, if appropriate, in the presence of an acid binding agent, or

p) compounds of the formula (I), in which

-A-(Q)ₖ-Z represents a group of the formula

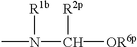

in which

R^{1b} has the above-mentioned meanings,

R^{2p} represents a hydrogen atom or C₁₋₄ haloalkyl and

R^{6p} represents a hydrogen atom or C₁₋₄ alkyl,

can be prepared by reacting 3,4-dichloro-isothiazole derivatives of the formula

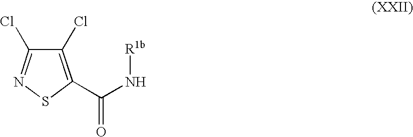

in which

R^{1b} has the above-mentioned meanings,

with compounds of the formula

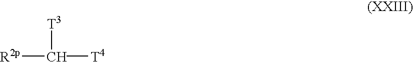

in which

R^{2p} has the above-mentioned meanings,

T³ represents hydroxy and

T⁴ represents C₁₋₄ alkoxy or

T³ and T⁴ together represent and oxo group,

in the presence of an inert diluent and, if appropriate, in the presence of an acid binding agent, or

q) compounds of the formula (I), in which

-A-(Q)ₖ-Z represents a group of the formula

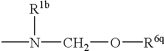

in which

R^{1b} has the above-mentioned meanings and

R^{6q} represents C₁₋₄ alkyl-carbonyl or benzoyl, which may be substituted by C₁₋₄ haloalkyl

can be prepared by reacting 3,4-dichloro-isothiazole derivatives of the formula

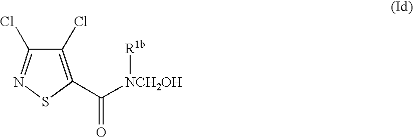

in which

R^{1b} has the above-mentioned meanings,

with chloro-substituted compounds of the formula

Cl—R^{6q} (XXIV)

in which

R^{6q} has the above-mentioned meanings,

in the presence of an inert diluent and, if appropriate, in the presence of an acid binding agent, or

r) compounds of the formula (I), in which

-A-(Q)ₖ-Z represents a group of the formula

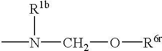

in which

R^{1b} has the above-mentioned meanings and

R^{6r} represents phenylcarbamoyl or C₁₋₄ haloalkyl-substituted phenylcarbamoyl

can be prepared by reacting 3,4-dichloro-isothiazole derivatives of the formula

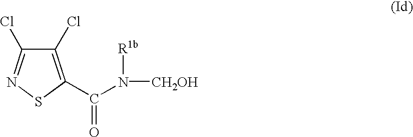

in which

R^{1b} has the above-mentioned meanings, with isocyanates of the formula

O═C═N—R^{r} (XXV)

in which

R^{r} represents phenyl or C₁₋₄ haloalkyl-substituted phenyl,

in the presence of an inert diluent and, if appropriate, in the presence of an acid binding agent and, if appropriate, in the presence of a base catalyst.

Finally, it has been found that the isothiazolecarboxylic acid derivatives of the formula (I) are outstandingly active as microbicides in agriculture and horticulture, particularly as fungicides for the direct control of plant diseases or for causing resistance in plants against plant pathogens.

Surprisingly, the isothiazolecarboxylic acid derivatives of the formula (I) according to the invention have a much better microbicidal activity than the already known compounds, which are structurally most similar and have the same type of action.

In the present context, “halogen” represents fluoro, chloro, bromo and iodo.

“Alkyl” represents straight-chain or branched groups, such as methyl, ethyl, n- or iso-propyl, n-, iso-, sec- or tert-butyl, n-pentyl, iso-pentyl, tert-amyl, pentan-3-yl, neopentyl, n-hexyl, n-heptyl, n-octyl, n-nonyl, n-decyl, n-undecyl, n-dodecyl, n-tridecyl, n-tetradecyl, n-pentadecyl and n-hexadecyl etc.

“Alkoxy” represents straight-chain or branched groups, such as methoxy, ethoxy, n- or iso-propoxy, n-, iso-, sec- or tert-butoxy etc.

“Cycloalkyl” represents cyclic alkyl groups and includes, for example, cyclopropyl, cyclobutyl, cyclopentyl, cyclohexyl, cycloheptyl, cyclooctyl etc.

“Haloalkyl” represents straight-chain or branched alkyl groups, which are substituted with one or more halogen atoms, preferably fluoro, chloro and/or bromo. As examples there may be mentioned difluoromethyl, trifluoromethyl, 2-fluoroethyl, 2-chloroethyl, 2-bromoethyl, 2,2,2-trifluoroethyl, 2,2,2-trichloroethyl, 3-chloropropyl, 3-bromopropyl, 1-chloropropan-2-yl, 1-bromopropan-2-yl, 1,3-difluoropropan-2-yl, 2,3-dibromopropyl, 2,2-dichloro-3,3,3-trifluoropropyl etc.

“Haloalkoxy” represents straight-chain or branched alkoxy groups, which are substituted with one or more halogen atoms, preferably fluoro, chloro and/or bromo.

As examples there may be mentioned difluoromethoxy, trifluoromethoxy, 2-fluoroethoxy, 2-chloroethoxy, 2-bromoethoxy, 2,2,2-trifluoroethoxy, 2,2,2-trichloroethoxy, 3-chloropropoxy etc.

“Alkenyl” represents straight-chain or branched groups and includes, for example, vinyl, allyl, isopropenyl, 1-propenyl, 1-butenyl, 2-butenyl, 3-butenyl, 1-methyl-1-propenyl, 2-methyl-1-propenyl etc.

“Aralkyl” represents groups of this type comprising a straight-chain or branched alkyl group and includes, for example, benzyl, 2-phenethyl, α-methylbenzyl, α,α-dimethylbenzyl, 2-phenylpropyl, 3-phenylpropyl, c-ethyl-benzyl etc.

A “5- to 7-membered heterocyclic groups” represents a 5-membered, 6-membered or 7-membered saturated heterocylic group, or a 5-membered unsaturated heterocyclic group, or a 5-membered or 6-membered aromatic heterocyclic group having 1-4 hetero atoms selected from nitrogen, oxygen and sulphur.

As “5-membered, 6-membered or 7-membered saturated heterocyclic groups” there may be mentioned monovalent groups, such as pyrrolidine, tetrahydrofuran, imidazolidine, pyrazolidine, oxazolidine, thiazolidine, piperidine, tetrahydropyran, piperazine, morpholine, 1,3-dioxolane, 1,3-dioxane, hexamethyleneimine etc. These heterocyclic groups may be substituted with one or more radicals selected from hydroxy, halogen (for example, fluoro, chloro, bromo etc.), oxo, thioxo, alkyl (for example, methyl, ethyl, n- or iso-propyl, n-, sec-, iso-, or tert-butyl etc.), alkoxy (methoxy, ethoxy, n- or iso-propoxy etc.), alkylthio (methylthio, ethylthio, n- or iso-propylthio etc.), alkoxyalkyl (methoxymethyl, ethoxymethyl etc.) or alkylthioalkyl (methylthiomethyl, ethylthiomethyl etc.), and in case of two or more substituents, they may be identical or different.

As “5-membered unsaturated heterocyclic groups” there may be mentioned monovalent groups, such as 2-pyrroline, 2-pyrazoline, 3-pyrazoline, 2-imidazoline, 2-oxazoline etc. These heterocyclic groups may be substituted with one or more radicals selected from hydroxy, halogen (for example, fluoro, chloro, bromo etc.), oxo, thioxo, alkyl (for example, methyl, ethyl, n- or iso-propyl, n-, sec-, iso-, or tert-butyl etc.), alkoxy (methoxy, ethoxy, n- or iso-propoxy etc.), alkylthio (for example, methylthio, ethylthio, n- or iso-propylthio etc.), alkoxyalkyl (for example, methoxymethyl, ethoxymethyl etc.) or alkylthioalkyl (for example, methylthiomethyl, ethylthiomethyl etc.), and in case of two or more substituents, they may be identical or different.

As “5- or 6-membered aromatic heterocyclic groups” there may be mentioned monovalent groups such as furan, pyrrole, thiophene, imidazole, pyrazole, oxazole, isoxazole, thiazole, isothiazole, 1,2,4-triazole, 1,3,4-thiadiazole, tetrazole, pyridine, pyridazine, pyrimidine, pyrazine etc.

These heterocyclic groups may be substituted with one or more radicals selected from hydroxy, oxo, thioxo, cyano, nitro, halogen (for example, fluoro, chloro, bromo etc.), alkyl (for example, methyl, ethyl, n- or iso-propyl, n-, sec-, iso-, or tert-butyl etc.), alkoxy (for example, methoxy, ethoxy, n- or iso-propoxy etc.), alkylthio (for example, methylthio, ethylthio, n- or iso-propylthio etc.), haloalkyl (for example, trifluoromethyl etc.), haloalkoxy (for example, trifluoromethoxy etc.), cyanoalkyl (for example, cyanomethyl, 1-cyanoethyl, 1-cyanopropyl etc.), alkoxycarbonyl (for example, methoxycarbonyl, ethoxycarbonyl etc.), alkoxyalkyl (for example, methoxymethyl, ethoxymethyl etc.) or alkylthioalkyl (for example, methylthiomethyl, ethylthiomethy etc.), and in case of two or more substituents, they may be identical or different.

A “benzo-condensed 5-membered or 6-membered heterocyclic group” represents a benzo-condensed hetero cyclic ring of any of the above-mentioned groups identified as “5- or 6-membered aromatic heterocyclic group” and includes monovalent groups selected from benzo[b]thiophene, benzothiazole, benzoimidazole, benzotriazole, quinoline etc. These benzo-condensed heterocyclic groups may be substituted with one or more radicals selected from cyano, nitro, halogen (for example, fluoro, chloro, bromo etc.), alkyl (for example, methyl, ethyl, n- or iso-propyl, n-, sec-, iso-, or tert-butyl etc.), alkoxy (for example, methoxy, ethoxy, n- or iso-propoxy etc.), alkylthio (for example, methylthio, ethylthio, n- or iso-propylthio etc.), alkoxyalkyl (for example, methoxymethyl, ethoxymethyl etc.) or alkylthioalkyl (for example, methylthiomethyl, ethylthiomethyl etc.), and in case of two or more substituents, they may be identical or different.

Formula (I) provides a general definition of the isothiazolecarboxylic acid derivatives according to the invention. Preferred compounds of the formula (I) are those, in which

A represents an oxygen atom, a sulphur atom or a group of the formula

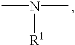

in which

R¹ represents a hydrogen atom, C₁₋₃ alkyl, cyclopentyl, cyclohexyl, phenyl or 2-hydroxyethyl,

Q represents a group selected from

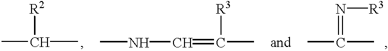

in which

R² represents a hydrogen atom, C₁₋₆ alkyl, haloalkyl with 1 to 3 carbon atoms and 1 to 5 fluorine, chlorine and/or bromine atoms, C₇₋₈ aralkyl or phenoxymethyl, which may be mono- or di-substituted by C₁₋₃ alkoxy-carbonyl, and

R³ represents phenyl, which may be substituted by 1 to 3 radicals selected from fluoro, chloro, bromo, C₁₋₃ alkyl, haloalkyl with 1 to 3 carbon atoms and 1 to 5 fluorine, chlorine and/or bromine atoms, C₁₋₃ alkoxy, haloalkoxy with 1 to 3 carbon atoms and 1 to 5 fluorine, chlorine and/or bromine atoms, phenoxy, benzyloxy, cyano and/or nitro, or may be mono-substituted by oxydimethylene, or represents naphthyl,

k represents 0 or 1, and

Z represents a 5-7-membered heterocyclic ring comprising 1 to 4 nitrogen atoms, wherein the heterocyclic ring may be substituted by up to 3 substituents selected from fluorine, chlorine, bromine, C₁₋₃ alkyl, methoxy, ethoxy, haloalkyl with 1 to 3 carbon atoms and 1 to 5 fluorine, chlorine and/or bromine atoms, cyclopropyl, cyclopentyl, C₃₋₄ alkenyl, phenyl and/or halophenyl comprising 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z represents a 5 or 6-membered heterocyclic ring comprising at least one nitrogen atom and one oxygen atom, or comprising at least one nitrogen atom and one sulphur atom, wherein each of the heterocyclic rings may be substituted by 1 to 3 substituents selected from C₁₋₃ alkyl and/or phenyl, and may also be substituted by 1 or 2 oxo groups, or

Z represents cyano or a group selected from 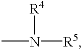 —O—R⁶, —S(O)ₘ—R⁷ and

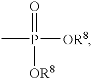

in which

R⁴ represents a hydrogen atom, C₁₋₃ alkyl, benzyl or phenyl, the last two radicals being optionally substituted by 1 to 3 radicals selected from fluorine, chlorine, methyl and/or ethyl, or

R⁴ represents tetrazol-5-yl-thiomethyl, which may be substituted by C₁₋₃ alkyl,

R⁵ represents formyl, C₁₋₄ alkylcarbonyl, 3,4-dichloroisothiazol-5-ylcarbonyl, C₁₋₂ alkylsulphonyl or phenylsulphonyl or

R⁵ represents phenylcarbonyl, optionally substituted by one to three radicals selected from fluorine, chlorine and/or C₁₋₄ alkyl,

R⁶ represents a hydrogen atom, C₁₋₃ alkyl, C₁₋₃ fluoroalkyl, or represents benzyl or phenyl, each of which may be substituted by 1 to 3 radicals selected from fluorine and/or chlorine, or represents acetyl or propionyl, or represents benzoyl or phenylcarbamoyl, each of which may be substituted by 1 to 3 radicals selected from haloalkyl with 1 to 3 carbon atoms and 1 to 3 fluorine, chlorine and/or bromine atoms,

R⁷ represents C₁₋₃ alkyl, benzyl or phenyl the last two radicals being optionally substituted by one to three radicals selected from C₁₋₃ alkyl, fluorine and/or chlorine, or

R⁷ represents tetrazol-5-yl or

R⁷ represents thiadiazol-2-yl optionally substituted by C₁₋₃ alkyl or phenyl, or

R⁷ represents 2-thiazoline-2-yl, C₁₋₂ alkylcarbonyl or benzoyl,

m represents o or 2, and

R⁸ represents methyl or ethyl,

or, in case

A represents

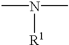

group,

then

R¹, Q and Z together with the nitrogen atom of the

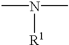

group may represent a 5- or 6-membered heterocyclic group comprising 1 to 3 nitrogen atoms and being optionally substituted by 1 to 3 radicals selected from C₁₋₄ alkyl, haloalkyl with 1 to 3 carbon atoms and 1 to 5 fluorine, chlorine and/or bromine atoms, hydroxy, oxo, hydroxymethyl and/or phenyl, which in turn may be substituted by 1 to 3 radicals selected from fluorine, chlorine and/or C₁₋₃ alkyl, or

-(Q)ₖ-Z represents a group selected from

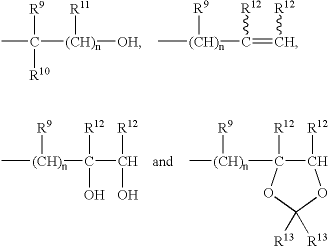

wherein

n represents 1 or 2,

R⁹ represents a hydrogen atom or C₁₋₃ alkyl,

R¹⁰ represents a hydrogen atom, hydroxymethyl or benzyl which may be substituted by 1 to 3 chlorine atoms,

R¹¹ represents a hydrogen atom, methyl, ethyl, n-propyl, isopropyl, tert-butyl or phenyl,

R¹² represents a hydrogen atom, C₁₋₃ alkyl or phenyl, or two of the R¹² radicals, together with the carbon atoms to which they are bonded, may form a 5- or 6-membered hydrocarbon ring, and

R¹³ represents a hydrogen atom, C₁₋₆ alkyl, cyclohexyl, 2-phenethyl, α-methylbenzyl, 2-cyclohexylethyl, C₁₋₃ alkoxy-C₁₋₃ alkyl or di(C₁₋₂ alkoxy)methyl, or the two R¹³ radicals, together with the carbon atom to which they are bonded, form a C₅₋₆ alicyclic ring which is optionally substituted by C₁₋₃ alkyl, or

-A-(Q)ₖ-Z represents —SH or a group of the formula

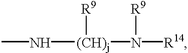

in which

R⁹ has the above-mentioned meanings,

R¹⁴ represents C₁₋₃ alkyl, cyclopentyl, cyclohexyl or hydroxy-substituted C₂₋₃ alkyl, and

j represents 2, 3 or 4,

or, in case

A represents

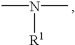

Q represents

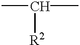

and

Z represents

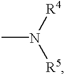

these

radicals together may represent a group of the formula

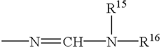

in which

R¹⁵ and R¹⁶ independently of one another represent C₁₋₃ alkyl or phenyl or

R¹⁵ and R¹⁶ together with the nitrogen atom, to which they are bonded, form a 5- or 6-membered heterocyclic group comprising at least one nitrogen atom or comprising at least one nitrogen atom and one oxygen atom,

with the proviso that

in case

Q represents a group of the formula

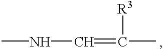

then

A represents

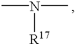

wherein

R¹⁷ represents a hydrogen atom or C₁₋₃ alkyl, and

Z represents cyano,

and in case

Q represents a group of the formula

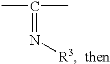

A represents —NH and

Z represents cyano

and in case

-(Q)ₖ-Z represents 2,3-dihydroxypropyl, then

A represents a sulphur atom or a group of the formula

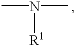

and in case

-(Q)ₖ-Z represents 2 hydroxyethyl and

A represents a group of the formula

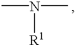

then

R¹ represents C₁₋₃ alkyl, cyclopentyl, cyclohexyl, phenyl or 2-hydroxyethyl

and in case

A represent a group of the formula

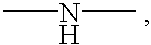

then

Q represents —CH₂— and

Z represents a group of the formula

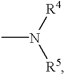

in which

R⁴ represents a hydrogen atom, benzyl or phenyl, the last two radicals being optionally substituted by 1 to 3 radicals selected from fluorine, chlorine, methyl and/or ethyl, and

R⁵ represents formyl,

and with the further provisio that

Z does not represent cyano or a group selected from 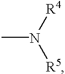 —OR⁶, —S(O)ₘ—R⁷ and

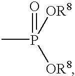

if

A is oxygen or sulphur and

k is o.

Particularly preferred are the compound of the formula (I), in which

A represents an oxygen atom, a sulphur atom or a group of the formula

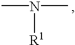

in which

R¹ represents a hydrogen atom, methyl, ethyl, n-propyl, iso-propyl, cyclopentyl, cyclohexyl, phenyl or 2-hydroxyethyl,

Q represents a group selected from

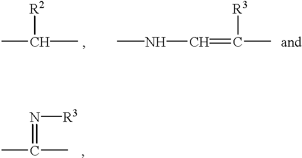

in which

R² represents a hydrogen atom, C₁₋₆ alkyl, trifluoromethyl, trichloromethyl, 2-phenylethyl or phenoxymethyl, which may be substituted by methoxycarbonyl, and

R³ represents phenyl, which may be substituted by 1 to 3 radicals selected from fluoro, chloro, methyl, ethyl, trifluoromethyl, methoxy, trifluoromethoxy, phenoxy, benzyloxy, cyano and/or nitro, or may be mono-substituted by oxydimethylene,

k represents o or 1, and

Z represents a 5- or 6-membered heterocyclic ring comprising 1 to 4 nitrogen atoms, wherein the heterocyclic ring may be substituted by up to 3 substituents selected from fluorine, chlorine, methyl, ethyl, propyl, methoxy, trifluoromethyl, cyclopropyl, cyclopentyl, 2-methyl-1-propenyl and/or phenyl, the latter radical being optionally substituted by 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z represents a 5- or 6-membered heterocyclic ring comprising at least one nitrogen atom and one oxygen atom, or comprising at least one nitrogen atom and one sulphur atom, wherein each of the heterocyclic rings may be substituted by 1 to 3 substitutents selected from methyl and/or phenyl, and may also be substituted by 1 or 2 oxo groups, or

Z represents cyano or a group selected from 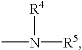 —O—R⁶, S(O)ₘ—R⁷ and

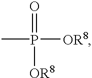

in which

R⁴ represents a hydrogen atom, methyl, ethyl, propyl, benzyl or phenyl, the last two radicals being optionally substituted by 1 to 3 radicals selected from fluorine, chlorine, methyl and/or ethyl, or

R⁴ represents tetrazol-5-yl-thiomethyl, which may be substituted by methyl,

R⁵ represents formyl, acetyl, pivaloyl, 3,4-dichloroisothiazol-5-ylcarbonyl, methylsulphonyl or phenylsulphonyl, or

R⁵ represents phenylcarbonyl, optionally substituted by 1 to 3 radicals selected from fluorine, chlorine and/or methyl,

R⁶ represents a hydrogen atom, methyl, ethyl, 2,2,3,3-tetrafluoropropyl, or represents benzyl or phenyl, each of which may be substituted by 1 to 3 radicals selected from fluorine and or chlorine, or

represents benzoyl or phenylcarbamoyl, each of which may be substituted by trifluormethyl, or

represents acetyl or propionyl,

R⁷ represents methyl, ethyl, phenyl or benzyl, the last two radicals being optionally substituted by 1 to 3 radicals selected from methyl, fluorine and/or chlorine, or

R⁷ represents tetrazol-5-yl or

R⁷ represents thiadiazol-2-yl optionally substituted by methyl or phenyl, or

R⁷ represents 2-thiazoline-2-yl, methylcarbonyl or benzoyl,

m represents o or 2, and

R⁸ represents methyl or ethyl,

or, in case

A represents a

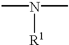

group,

then

R¹, Q and Z together with the nitrogen atom of the

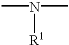

group may represent a 5- or 6-membered heterocyclic group comprising 1 or 2 nitrogen atoms and being optionally substituted by 1 to 3 radicals selected from methyl, ethyl, n-propyl, iso-propyl, tert-butyl, trifluoromethyl, hydroxy, oxo, hydroxymethyl and/or phenyl, which in turn may be substituted by 1 to 3 radicals selected from fluorine, chlorine and/or methyl, or

-(Q)ₖ-Z represents a group selected from

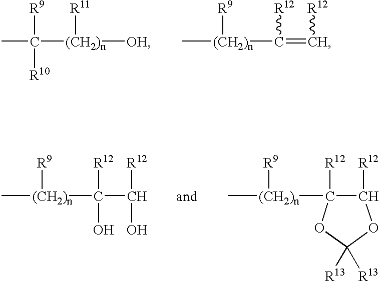

wherein

n represents 1 or 2,

R⁹ represents a hydrogen atom, methyl or ethyl,

R¹⁰ represents a hydrogen atom, hydroxymethyl or benzyl, which may be substituted by chlorine,

R¹¹ represents a hydrogen atom, methyl, ethyl, n-propyl, iso-propyl, tert-butyl or phenyl,

R¹² represents a hydrogen atom, methyl or phenyl, or two of the R¹² radicals, together with the atoms to which they are bonded, may form a 5- or 6-membered hydrocarbon ring, and

R¹³ represents a hydrogen atom, C₁₋₄ alkyl, cyclohexyl, 2-phenethyl, α-methylbenzyl, 2-cyclohexylethyl, ethoxymethyl, 2-ethoxyethyl or dimethoxymethyl, or the two R¹³ radicals, together with the carbon atom to which they are bonded, form a C₅₋₆ alicyclic ring which is optionally substituted by C₁₋₃ alkyl, or

-A-(Q)ₖ-Z represents —SH or a group of the formula

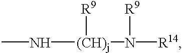

in which

R⁹ has the above-mentioned meanings,

R¹⁴ represents methyl, ethyl, cyclopentyl, cyclohexyl or hydroxyethyl, and

j represents 2 or 3,

or, in case

A represents

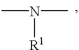

Q represents

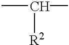

and

Z represents

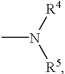

these

radicals together may represent a group of the formula

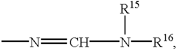

in which

R¹⁵ and R¹⁶ independently of one another represent methyl, ethyl or phenyl or

R¹⁵ and R¹⁶ together with the nitrogen atom, to which they are bonded, form a 5- or 6-membered heterocyclic group comprising at least one nitrogen atom or comprising at least one nitrogen atom and one oxygen atom,

with the proviso that

in case

Q represents a group of the formula

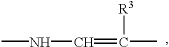

then

A represents —NH— or

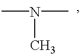

and

Z represents cyano,

and in case

Q represents a group of the formula

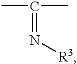

then

A represents —NH and

Z represents cyano

and in case

-(Q)ₖ-Z represents 2,3-dihydroxypropyl, then

A represents a sulphur atom or a group of the formula

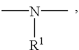

and in case

-(Q)ₖ-Z represents 2 hydroxyethyl and

A represents a group of the formula

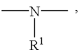

then

R¹ represents methyl, cyclopentyl, cyclohexyl, phenyl or 2-hydroxyethyl

and in case

A represents a group of the formula

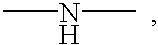

then

Q represents —CH₂ and

Z represents a group of the formula

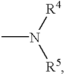

in which

R⁴ represents a hydrogen atom, benzyl or phenyl, the last two radicals being optionally substituted by 1 to 3 radicals selected from fluorine, chlorine, methyl and/or ethyl, and

R⁵ represents formyl,

and with the further proviso that

Z does not represent cyano or a group selected from 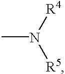 —OR⁶, —S(O)ₘ—R⁷ and

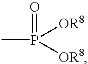

if

A is oxygen or sulphur and

k is o.

If 3,4-dichloro-isothiazole-5-carboxamide and N-benzyl-N-hydroxymethyl-formamide are used as starting materials, process (a) according to the invention can be illustrated by the following reaction scheme.

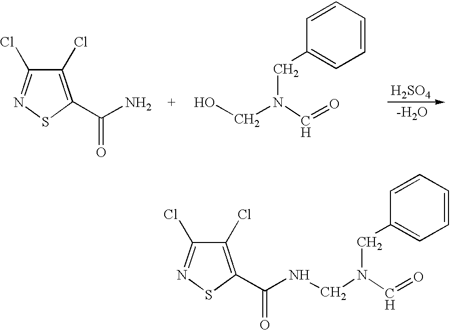

If N-chloromethyl-3,4-dichloroisothiazole-5-carboxamide and sodium salt of 4-chloro-thiophenol are used as starting materials, process (b) according to the invention can be illustrated by the following formula scheme.

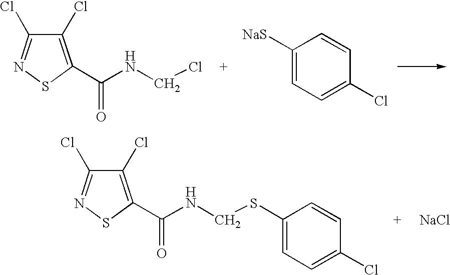

If N-chloromethyl-N-methyl-3,4-dichloro-isothiazole-5-carboxamide and triethylphosphite are used as starting materials, process (c) according to the invention can be illustrated by the following formula scheme.

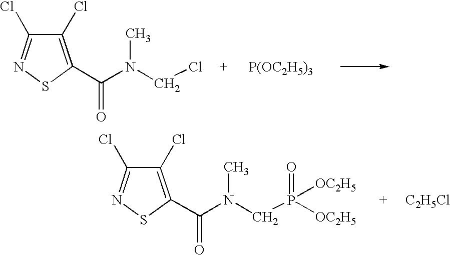

If 3,4-dichloro-isothiazole-5-carboxamide and chloromethyl-methylether are used as starting materials, process (d) according to the invention can be illustrated by the following formula scheme.

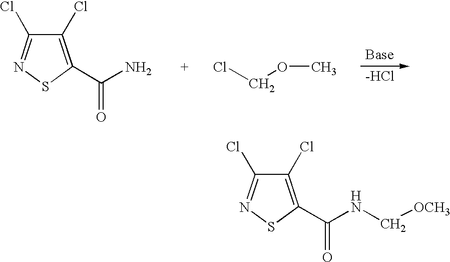

If 3,4-dichloro-isothiazole-5-carboxamide, butyraldehyde and benzotriazole are used as starting materials, process (e) according to the invention can be illustrated by the following formula scheme.

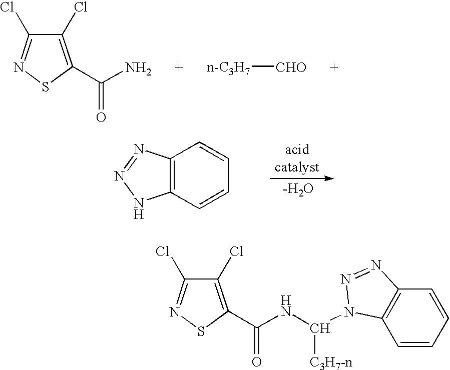

If 3,4-dichloro-isothiazole-5-carbonyl chloride and N-(1-aminobutyl)-acetamide are used as starting materials, process (f according to the invention can be illustrated by the following formula scheme.

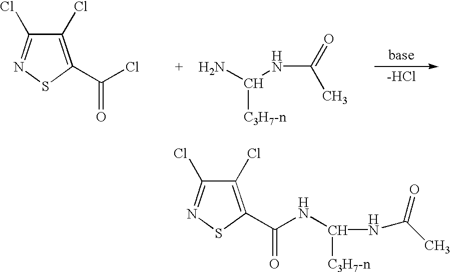

If 3,4-dichloro-isothiazole-5-methyl carboxylate and 3-amino-1,2-propandiol are used as starting materials, process (g) according to the invention can be illustrated by the following formula scheme.

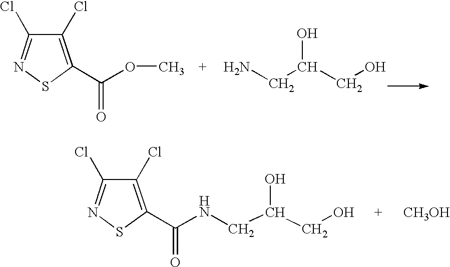

If 3,4-dichloro-isothiazole-5-carbohydrazide and diethyl (4-chloro-benzylidene)malonate are used as starting materials, process (h) according to the invention can be illustrated by the following formula scheme.

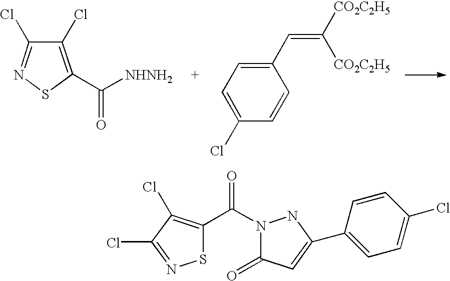

If 3,4-dichloro-isothiazole-5-carbohydrazide and 2-methyl-3-phenyl-2-propenoyl chloride are used as starting materials, process (i) according to the invention can be illustrated by the following formula scheme.

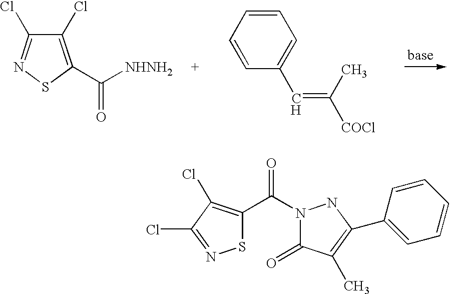

If 3,4-dichloro-isothiazole-5-carbohydrazide and 2-formyl-2-phenylacetonitrile are used as starting materials, process (j) according to the invention can be illustrated by the following formula scheme.

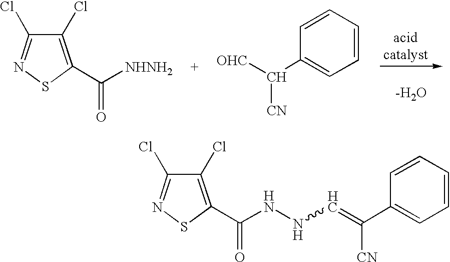

If N-phenylthiomethyl-3,4-dichloro-isothiazole-5-carboxamide and m-chloroperbenzoic acid are used as starting materials, process (k) according to the invention can be illustrated by the following formula scheme.

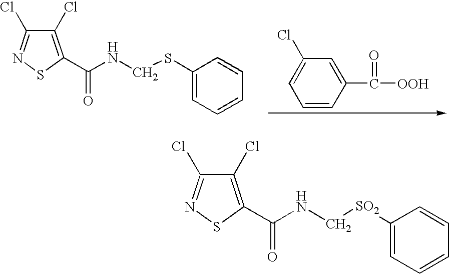

If 3,4-dichloro-isothiazole-5-carboxamide and N,N-dimethylformamide dimethylacetal are used as starting materials, process (l) according to the invention can be illustrated by the following formula scheme.

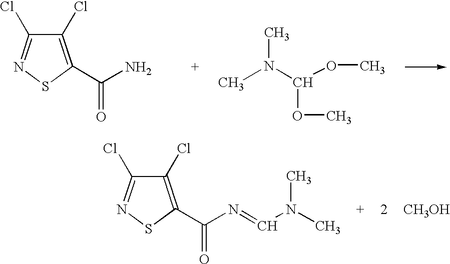

If N-alkyl-N-phenyl-3,4-dichloroisothiazole-5-carboxamide is used as starting materials and osmium (VIII) oxide is used as oxidizing agent, process (m) according to the invention can be illustrated by the following formula scheme.

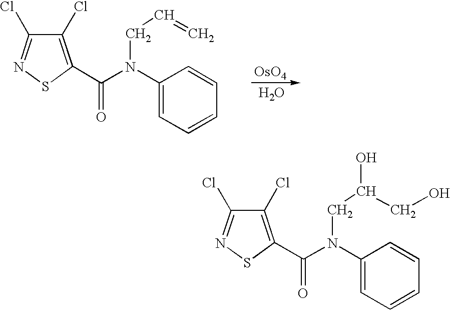

If N-(2,3-dihydroxypropyl)-3,4-dichloro-isothiazole-5-caboxamide and acetone dimethylacetal are used as starting materials, process (n) according to the invention can be illustrated by the following formula scheme.

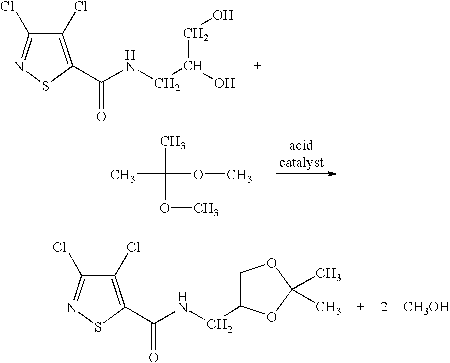

If 3,4-dichloro-isothiazole-5-carboxamide and 2-chloro-2-(4-trifluoromethyl-phenylimino)-acetonitrile are used as starting materials, process (o) according to the invention can be illustrated by the following formula scheme.

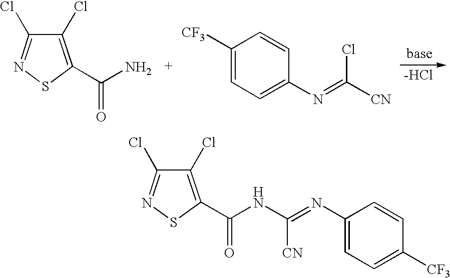

If 3,4-dichloro-isothiazole-5-carboxamide and trifluoroacetaldehyde hemi-ethylacetal are used as starting materials, process (p) according to the invention can be illustrated by the following formula scheme.

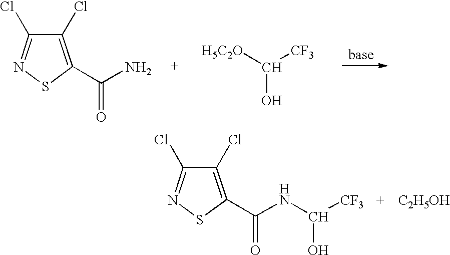

If N-hydroxymethyl-3,4-dichloro-isothiazole-5-carboxamide and 4-trifluoromethyl-benzoyl chloride are used as starting materials, process (q) according to the invention can be illustrated by the following formula scheme.

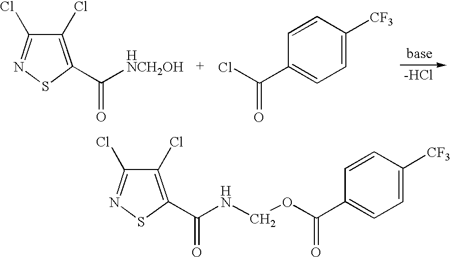

If N-hydroxymethyl-3,4-dichloro-isothiazole-5-carboxamide and 4-trifluoromethylphenyl isocyanate are used as starting materials, process (r) according to the invention can be illustrated by the following formula scheme.

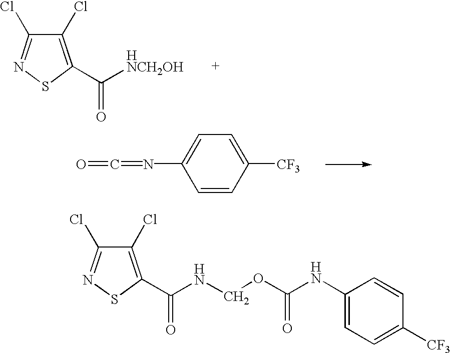

Formula (II) characterizes the 3,4-dichloro-isothiazole-5-carboxamide, which is required as starting material for carrying out processes (a), (e), (l), (o) and (p) according to the invention. The 3,4-dichloro-isothiazole-5-carboxamide is known (see U.S. Pat. No. 5,240,951).

Formula (III) provides a definition of the formylamine, which is also required as starting material for carrying out process (a) according to the invention. This compound is already known (see Synth. Commun. 18 (1988), 425-432). The chemical name of the compound of the formula (III) is N-benzyl-N-hydroxymethylformamide.

Formula (IV) provides a general definition of the isothiazole derivatives, which are required as starting materials for carrying out process (b) according to the invention. In this formula, R^{1b} preferably represents a hydrogen atom or C₁₋₃ alkyl, R^{2b} preferably represents a hydrogen atom or haloalkyl with 1 to 3 carbon atoms and 1 to 5 fluorine and/or chlorine atoms, and X preferably represents chloro.

The isothiazole derivatives of the formula (IV) have not yet been described in the literature. They can be prepared by reacting isothiazole derivatives of the formula

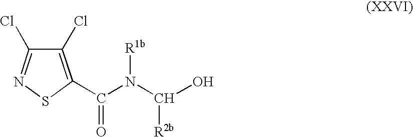

wherein

R^{1b} and R^{2b} have the above-mentioned meanings

with halogenating agents, such as thionyl chloride, phosphorus oxychloride, thionyl bromide, phosphorus oxybromide and so on.

The compounds of the above-mentioned formula (XXVI) are compounds included in the aforementioned formula (I) of the present invention and can be easily prepared from the known compounds of the aforementioned formula (XXII) according to the above-mentioned preparation process (p).

The isothiazole derivatives of the formula (IV), in which R^{1b} and R^{2b} both represent a hydrogen atom can be easily prepared from the compound of the aforementioned formula (XXII) according to the process described in Tetrahedron Letters Vol. 38, p. 7107-7110 (1994).

The following compounds may be mentioned as examples of isothiazole derivatives of the formula (IV):

N-Chloromethyl-3,4-dichloro-5-isothiazolecarboxamide,

N-chloromethyl-N-methyl-3,4-dichloro-5-isothiazolecarboxamide,

N-(1-chloromethyl-2,2,2-trifluoroethyl)-3,4-dichloro-5-isothiazolecarboxamide.

Formula (V) provides a general definition of the compounds, which are required as reaction components for carrying out process (b) according to the invention. In this formula, Z^{b} represents a group of the formula

—OR⁶, —SR⁷, —SO₂—R⁷ and

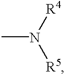

wherein

R⁴, R⁵, R⁶ and R⁷ preferably have those meanings, which have already been mentioned as preferred for these radicals.

M preferably represents a hydrogen atom, lithium or sodium.

The following compounds may be mentioned as examples of substances of the formula (V):

4-chlorophenol,

thiophenol,

piperidine,

sodium benzenesulfinate.

The compounds of the formula (V) are known.

Formula (VIa) provides a general definition of the isothiazole derivatives, which are required as starting materials for carrying out process (c) according to the invention. The compounds of this type have already been described in conjuncture with process (b) according to the invention.

Formula (VI) provides a general definition of the phosphorous compounds, which are required as reaction components for carrying out process (c) according to the invention. In this formula, R⁸ preferably has those meanings, which have already been mentioned as preferred for this radical.

The phosphorus compounds of the formula (VI) are already known. Triethyl phosphite may be mentioned as an example of a phosphorous compound of the formula (VI).

Formula (VII) provides a general definition of the isothiazole derivatives, which are required as starting materials for carrying our process (d) according to the invention.

In this formula,

A^{d} represents a sulphur atom or a group of the formula

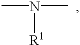

wherein

R¹ preferably has those meanings, which already been mentioned as preferred for this radical.

The compound of the formula (VII) in which A^{d} represents a sulphur atom, is novel and is included in formula (I). It can be prepared by process (f) according to the invention.

The compounds of the formula (VII), in which A^{d} represents

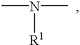

are known or can be prepared by known methods (see U.S. Pat. No. 5,240,951).

The following compounds may be mentioned as examples of isothiazole derivatives of the formula (VII):

3,4-Dichloro-5-isothiazolecarboxamide,

N-methyl-3,4-dichloro-5-isothiazolecarboxamide.

Formula (VIII) provides a general definition of the chloromethyl compounds, which are required as reaction components for carrying out process (d) according to the invention. In this formula,

Z^{d} preferably represents a 5-7-membered heterocyclic ring comprising 1 to 4 nitrogen atoms, wherein the heterocyclic ring may be substituted by up to 3 substituents selected from fluorine, chlorine, bromine, C₁₋₃ alkyl, methoxy, ethoxy, haloalkyl with 1 to 3 carbon atoms and 1 to 5 fluorine, chlorine, and/or bromine atoms, cyclopropyl, cyclopentyl, C₃₋₄ alkenyl, phenyl and/or halophenyl comprising 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z^{d} preferably represents a 5 or 6-membered heterocyclic ring comprising at least one nitrogen atom and one oxygen atom, or comprising at least one nitrogen atom and one sulphur atom, wherein each of the heterocyclic rings may be substituted by one or more substituents selected from C₁₋₃ alkyl and/or phenyl, and may also be substituted by 1 or 2 oxo groups, or

Z^{d} represents cyano or a group selected from 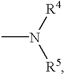 —OR⁶ and —SR⁷,

wherein

R⁴, R⁵, R⁶ and R⁷ preferably have those meanings, which have already been mentioned as preferred for these radicals.

Particularly preferred are the chloromethyl compounds of the formula (VIII) in which

Z^{d} represents a 5- or 6-membered heterocyclic ring comprising 1 to 4 nitrogen atoms, wherein the heterocyclic ring may be substituted up to 3 substituents selected from fluorine, chlorine, methyl, ethyl, propyl, methoxy, trifluoromethyl, cyclopropyl, cyclopentyl, 2-methyl-1-propenyl and/or phenyl, the latter radical being optionally substituted by 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z^{d} represents a 5 or 6-membered heterocyclic ring comprising at least one nitrogen atom and one oxygen atom, or comprising at least one nitrogen atom and one sulphur atom, wherein each of the heterocyclic rings may be substituted by 1 to 3 substituents selected from methyl and/or phenyl, and may also be substituted by 1 or 2 oxo groups, or

Z^{d} represents a group selected from 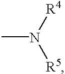 —OR⁶ and —SR⁷,

wherein

R⁴, R⁵, R⁶ and R⁷ particularly preferably have those meanings, which have already been mentioned as particularly preferred for these radicals.

The following compounds may be mentioned as examples of chloromethyl compounds of the formula (VIII):

N-Chloromethyl-N-methylacetamide,

chloromethyl methyl ether,

chloromethyl methyl thioether.

The chloromethyl compounds of the formula (VII) are known or can be prepared according to known processes (see Tetrahedron Letters 38-(1994) 7107-7110).

Formula (IX) provides a general definition of the formyl compounds, which are required as starting materials for carrying our process (e) according to the invention. In this formula, R² preferably has those meanings, which have already been mentioned as preferred for this radical.

The following compounds may be mentioned as examples of formyl compounds of the formula (IX):

Formaldehyde

n-butyraldehyde

The formyl compounds of the formula (IX) are known.

1H-Benzotriazole of the formula (X) is required as reaction component for carrying out process (e) according to the invention. This compound is known too.

3,4-Dichloro-isothiazole-5-carbonylchloride of the formula (XI) is required as starting material for carrying out process (f) according to the invention. This compound is also known (see JP-A 59024-1993).

Formula (XII) provides a general definition of the compounds, which are also required as starting materials for carrying out process (f) according to the invention. In this formula, M preferably has those meanings, which have already been mentioned as preferred for this radical.

Y¹ represents —SH or a group selected from

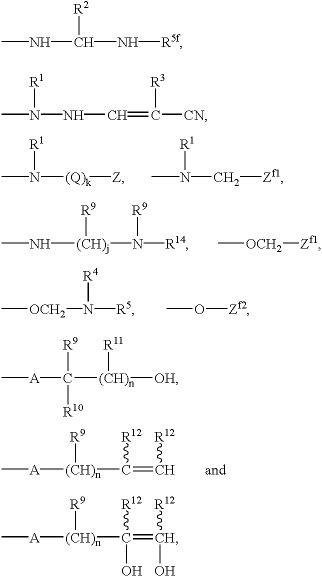

in which

A, Q, Z, j, k, n, R¹, R², R³, R⁴, R⁵, R⁹, R¹⁰, R¹¹, R¹² and R¹⁴ preferably have those meanings, which have already been mentioned as preferred for these radicals and indices.

Z^{f1} preferably represents a 5-7-membered heterocyclic ring comprising 1 to 4 nitrogen atoms, wherein the heterocyclic ring may be substituted by up to 3 substituents selected from fluorine, chlorine, bromine, C₁₋₃ alkyl, methoxy, ethoxy, haloalkyl with 1 to 3 carbon atoms and 1 to 5 fluorine, chlorine and/or bromine atoms, cyclopropyl, cyclopentyl, C₃₋₄ alkenyl, phenyl and/or halophenyl comprising 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z^{f1} preferably represents a 5 to 6-membered heterocyclic ring comprising at least one nitrogen atom and one oxygen atom, or comprising at least one nitrogen atom and one sulphur atom, wherein each of the heterocyclic rings may be substituted by 1 to 3 substituents selected from C₁₋₃ alkyl and/or phenyl, and may also be substituted by 1 or 2 oxo groups.

Z^{f2} preferably represents a 5-membered heterocyclic group comprising 1 or 2 nitrogen atoms, which heterocycle may be substituted by up to 3 radicals selected from C₁₋₃ alkyl and/or oxo,

R^{5f} preferably represents formyl, C₁₋₄ alkylcarbonyl or represents phenylcarbonyl, optionally substituted by 1 to 3 radicals selected from fluorine, chlorine and/or C₁₋₄ alkyl.

Z^{f1} particularly preferably represents a 5- or 6-membered heterocyclic ring comprising 1 to 4 nitrogen atoms, wherein the heterocyclic ring may be substituted by up to 3 substituents selected from fluorine, chlorine, methyl, ethyl, propyl, methoxy, trifluoromethyl, cyclopropyl, cyclopentyl, 2-methyl-1-propenyl and/or phenyl, the latter radical being optionally substituted by 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or

Z^{f1} particularly preferably represents a 5 or 6-membered heterocylic ring comprising at least one nitrogen atom and one oxygen atom, or comprising at least one nitrogen atom and one sulphur atom, wherein each of the heterocyclic rings may be substituted by 1 to 3 substituents selected from methyl and/or phenyl, and may also be substituted by 1 or 2 oxo groups.

Z^{f2} particularly preferably represents a 5-membered heterocyclic group comprising 1 or 2 nitrogen atoms, which heterocycle may be substituted by up to 3 radicals selected from methyl, ethyl, n-propyl and/or oxo.

R^{5f} particularly preferably represents formyl, acetyl, pivaloyl, or

represents phenylcarbonyl, optionally substituted by 1 to 3 radicals selected from fluorine, chlorine and/or methyl.

The following compounds may be mentioned as examples of compounds of the formula (XII):

Hydrogen sulfide,

N-(1-aminobutyl)acetamide,

N-(2-cyano-2-phenylvinyl)-N-methylhydrazine,

5-phenyl-2,3-dihydro-1H-pyrazolone,

2-aminomethyl-1-methylpyrrolidine,

N-allylaniline,

N,N-dimethylethylenediamine,

1-hydroxymethyl-3,5-dimethylpyrazole,

1-hydroxymethyl-4-methyl-3-trifluoromethyl-4,5-dihydro-1H-1,2,4-triazol-5-one,

3-hydroxy-1-methylpyrrolidin-2,5-dione,

b is (2-hydroxyethyl)amine,

allylamine,

3-amino-1,2-propanediol.

The compounds of the formula (XII) are known or can be prepared by known processes (see J. Org. Chem. 55 (1990), 2206-2214).

Formula (XIII) provides a general definition of the 3,4-dichloro-isothiazole-5-carboxylic acid esters, which are required as starting materials for carrying out process (g) according to the invention. In this formula R⁹ preferably represents methyl or ethyl. Thus, the following compounds may be mentioned as examples of esters of the formula (XIII):

Methyl 3,4-dichloro-5-isothiazolecarboxylate,

ethyl 3,4-dichloro-5-isothiazolecarboxylate.

The esters of the formula (XIII) ate known (see JP-A 59024-1993).

Formula (XIV) provides a general definition of the compounds, which are required as reaction components for carrying out process (g) according to the invention. In this formula

Y² represents a group selected from

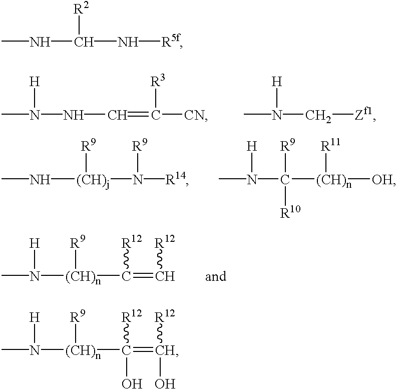

in which

Z^{f1}, j, n, R², R³. R⁹, R¹⁰, R¹¹, R¹², R¹⁴ and R^{5f} preferably have the meanings, which have already been mentioned as preferred for these radicals and indices.

The following compounds may be mentioned as examples of compounds of the formula (XIV):

2-Aminomethyl-1-methylpyrrolidine,

N-methylethylenediamine,

2-aminopropanol,

3-amino-1,2-propanediol,

allylamine.

As already mentioned above, the compounds of the formula (XIV) are known or can be prepared by known processes.

3,4-dichloro-isothiazole-5-carbohydrazide of the formula (XV) is required as starting material for carrying out processes (h), (i) and (j) according to the invention. This compound is already known (see DE-A 2 634 053).

Formula (XVI) provides a general definition of the compounds, which are required as reaction components for carrying out process (h) according to the invention. In this formula

R^{h1} preferably represents phenyl, which may be substituted by 1 to 3 radicals selected from fluorine, chlorine and/or C₁₋₃ alkyl,

R^{h2} preferably represents methyl or ethyl and

R^{h3} preferably represents cyano, methoxycarbonyl or ethoxycarbonyl.

Diethyl 4-chlorophenyl-ethylidene-malonate may be mentioned as an example of a compound of the formula (XVI).

The compounds of the formula (XVI) are known or can be prepared by known processes (see Organic Reactions 15, 204-599).

Formula (XVII) provides a general definition of the compounds, which are required as reaction components for carrying out process (i) according to the invention. In this formula

Rⁱ¹ preferably represents a hydrogen atom C₁₋₄ alkyl or represents phenyl, which may be substituted by 1 to 3 radicals selected from fluorine, chlorine and C₁₋₃ alkyl, and

Rⁱ² preferably represents a hydrogen atom or C₁₋₄ alkyl.

2-Methyl-3-phenyl-2-propenoyl chloride may be mentioned as an example of the compounds of the formula (XVII).

The compounds of the formula (XVII) are known or can be prepared by known processes (see SHIN JIKKEN KAGAKU KOUZA (New Lecture of Experimental Chemistry), Vol. 14, p. 1104-1120, published by Maruzen).

Formula (XVIII) provides a general definition of the compounds, which are required as reaction components for carrying out process (j) according to the invention. In this formula, R³ preferably has those meanings, which have already been mentioned as preferred for this radical.

2-Formyl-2-phenylacetonitrile may be mentioned as an example of the compounds of the formula (XVIII).

The compounds of the formula (XVIII) are known or can be prepared by known processes (see U.S. Pat. No. 4,209,621).

For carrying out process (k) according to the invention, isothiazolecarboxylic acid derivatives of the formula (Ia) are required as starting materials. In this formula, R¹, R² and R⁷ preferably have those meanings, which have already been mentioned as preferred for these radicals.

N-Phenyl-mercaptomethyl-3,4-dichloro-isothiazole-5-carboxamide may be mentioned as an example of the compounds of the formula (Ia).

The compounds of the formula (Ia) can be prepared by processes (b) and (d) according to the invention.

Suitable oxidizing agents for carrying out process (k) according to the invention are hydrogen peroxide and m-chloro-perbenzoic acid.

Formula (XIX) provides a general definition of the compounds, which are required as reaction components for carrying out process (l) according to the invention. In this formula, R¹⁵ preferably has those meanings, which have already been mentioned as preferred for this radical. T¹ preferably represents methoxy or ethoxy.

Dimethylformamide dimethylacetal may be mentioned as an example of the compounds of the formula (XIX).

The compounds of the formula (XIX) are known or can be prepared by known processes (see Chem. Ber. 89 (1956), 2060; Chem. Ber. 96 (1963), 1350; Chem. Ber. 101 (1968), 41; Chem. Ber. 104 (1971), 3475 and Liebigs Ann. Chem. 641 (1961), 1).

For carrying out process (m) according to the invention, isothiazolecarboxylic acid derivatives of the formula (Ib) are required as starting materials. In this formula, A, R⁹, R¹² and n preferably have those meanings, which have already been mentioned as preferred for these radicals and this index.

N-Allyl-N-phenyl-3,4-dichloro-isothiazole-5-carboxamide may be mentioned as an example of the compounds of the formula (Ib).

The compounds of the formula (Ib) can be prepared by process (f) according to the invention.

Suitable oxidizing agents for carrying out process (m) according to the invention are substances, which can provide oxygen to C═C double bonds. A preferred oxidizing agent of this type is osmium (VIII) oxide.

For carrying out process (n) according to the invention, isothiazolecarboxylic acid derivatives of the formula (Ic) are required as starting materials. In this formula, A, R⁹, R¹², and n preferably have those meanings, which have already been mentioned as preferred for these radicals and this index.

N-(2,3-Dihydroxypropyl)-3,4-dichloro-isothiazole-5-carboxamide may be mentioned as an example of the compounds of the formula (Ic).

The compounds of the formula (Ic) can be prepared by processes (f) and (m) according to the invention.

Formula (XX) provides a general definition of the carbonyl derivatives, which are also required as starting materials for carrying out process (n) according to the invention. In this formula, R¹³ preferably has those meanings, which have already been mentioned as preferred for this radical. T² preferably represents methoxy or ethoxy, or the two T²-radicals together represent an oxo group.

The following compounds may be mentioned as examples of carbonyl derivatives of the formula (XX):

Acetone dimethylacetal,

cyclohexanone.

The carbonyl derivatives of the formula (XX) are known.

Formula (XXI) provides a general definition of the cyano compounds, which are required as reaction components for carrying out process (o) according to the invention. In this formula, R³ preferably has those meanings, which have already been mentioned as preferred for this radical.

N-(chloro-cyano-methylidene)-4-trifluoromethyl-aniline may be mentioned as an example of the cyano compounds of the formula (XXI).

The cyano compounds of the formula (XXI) are known or can be prepared by known processes (see J. Chem. Soc., Perkin Trans. 1 (1997), 201).

Formula (XXII) provides a general definition of the 3,4-dichloro-isothiazole derivatives, which are required as starting materials for carrying out process (p) according to the invention. In this formula, R^{1b} preferably has those meanings, which have already been mentioned as preferred for this radical.

The 3,4-dichloro-isothiazole derivatives of the formula (XXII) are known or can be prepared by known processes (see U.S. Pat. No. 5,240,951).

Formula (XXIII) provides a general definition of the compounds, which are required as reaction components for carrying out process (p) according to the invention. In this formula, R^{2p} preferably represents haloalkyl with 1 to 3 carbon atoms and 1 to 5 fluorine and/or chlorine atoms or represents a hydrogen atom,

T³ represents hydroxy and

T⁴ preferably represents methoxy or ethoxy or

T³ and T⁴ together represent an oxo group.

The following compounds may be mentioned as examples of compounds of the formula (XXIII):

Paraformaldehyde,

trifluoroacetaldehyde hemiethylacetal.

The compounds of the formula (XXIII) are known or can be prepared by known processes.

For carrying out processes (q) and (r) according to the invention, 3,4-dichloroisothiazole derivatives of the formula (Id) are required as starting materials. In this formula, R^{1b} preferably has those meanings, which have already been mentioned as preferred for this radical.

The compounds of the formula (Id) can be prepared by process (d) according to the invention.

Formula (XXIV) provides a general definition of the compounds, which are required as reaction components for carrying our process (q) according to the invention. In this formula, R^{6q} preferably represents alkylcarbonyl with 1 to 3 carbon atoms in the alkyl group or represents benzoyl, which can be substituted by 1 to 3 substituents selected from haloalkyl with 1 to 3 carbon atoms and 1 to 5 fluorine and/or chlorine atoms.

The chloro-substituted compounds of the formula (XXIV) are known or can be prepared by known processes.

Formula (XXV) provides a general definition of the isocyanates, which are required as reaction components for carrying out process (r) according to the invention. In this formula, R^{r} preferably represents phenyl, which may be substituted by 1 to 3 substituents selected from haloalkyl with 1 to 3 carbon atoms and 1 to 5 fluorine and/or chlorine atoms.

The isocyanates of the formula (XXV) are known or can be prepared by known processes.

Suitable diluents for carrying out process (a) according to the invention are aliphatic carboxylic acids, such as acetic acid etc.

Suitable catalysts for carrying out process (a) according to the invention are all commonly used acid catalysts. As examples of such catalysts there may be mentioned mineral acids, such as sulfuric acid.

When carrying out process (a) according to the invention, the reaction temperatures can be varied within a substantially wide range. The reaction is generally carried out at a temperature between about 0° C. and about +150° C., preferably between about 10° C. and about 130° C.

Process (a) according to the invention is generally carried out under atmospheric pressure but, if desired, can also be carried out under elevated or reduced pressure.

When carrying out process (a) according to the invention, in general 1 mole of 3,4-dichloro-isothiazole-5-carboxamide of the formula (II) is reacted with 1 to 1.5 moles of the compound of the formula (III) in the presence of a diluent, such as acetic acid, and in the presence of a catalyst, such as sulfuric acid.

Process (b) according to the invention can be carried out in the presence of a diluent. Suitable diluents are all costomary inert organic solvents. The following can preferably be used: aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitrites, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for carrying out process (b) according to the invention are all customary inorganic and organic bases. Preferred as inorganic bases are hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide etc.; inorganic alkali metal amides, for example, lithium amide, sodium amide, potassium amide etc., and preferred organic bases are alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

Process (b) according to the invention can also be conducted in the presence of a phase-transfer catalyst. Suitable diluents in this case are water; aliphatic, alicyclic and aromatic hydrocarbons (which may optionally be chlorinated), for example, pentane, hexane, cyclohexane, petroleum ether, ligroine, benzene, toluene, xylene, dichloromethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, propionitrile, acrylonitrile etc.

As examples of phase-transfer catalysts there can be mentioned quaternary ions, for example, tetramethylammonium bromide, tetrapropylammonium bromide, tetrabutylammonium bromide, tetrabutylammonium hydrogen sulfate, tetrabutylammonium iodide, trioctylmethylammonium chloride, benzyltriethylammonium bromide, butylpyridinium bromide, heptylpyridinium bromide, benzyltriethylammonium chloride etc.; crown ethers, for example, dibenzo-18-crown-6, dicyclohexyl-18-crown-6,18-crown-6 etc.; cryptands, for example, [2.2.2]-cryptate, [2.1.1]-cryptate, [2.2.1]-cryptate, [2.2.B]-cryptate, [20202S]-cryptate, [3.2.2]-cryptate etc.

When carrying out process (b) according to the invention, the reaction temperatures can be varied within a substantially wide range. The reaction is generally carried out at a temperature between about −80° C. and about +200° C., preferably between about −10° C. and about +130° C.

Process (b) according to the invention is generally carried out under atmospheric pressure but, if desired, can also be carried out under elevated or reduced pressure.

When carrying out process (b) according to the invention, in general 1 mole of an isothiazole derivative of the formula (IV) is reacted with 1 to 1.5 moles of a compound of the formula (V) in the presence of a diluent, such as dimethylformamide, and in the presence of 1 to 1.5 moles of an acid-binding agent, such as sodium hydride.

In a particular variant, process (b) according to the invention can also be carried out by starting form a compound of the formula (XXVI), converting same into a compound of the formula (IV) and reacting it without prior isolation with a compound of the formula (V).

Suitable diluents for conducting process (c) according to the invention are all customary inert organic solvents. The following can preferably be used: aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitrites, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

When carrying out process (c) according to the invention, the reaction temperatures can be varied within a substantially wide range. The reaction is generally carried out at a temperature between about 0° C. and about 200° C., preferably between about 20° C. and about 150° C.

Process (c) according to the invention is generally carried out under atmospheric pressure but, if desired, can also be carried out under elevated or reduced pressure.

When carrying out process (c) according to the invention, in general 1 mole of an isothiazole derivative of the formula (IVa) is reacted with 1 to 50 moles of a phosphorous compound of the formula (VI).

Suitable diluents for conducting process (d) according to the invention are all customary inert organic solvents. The following can preferably be used:

aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for conducting process (d) according to the invention are all customary inorganic and organic bases. The following can preferably be used: Inorganic bases, such as, hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide etc.; inorganic alkali metal amides, for example, lithium amide, sodium amide, potassium amide etc.; organic bases, such as alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying our process (d) according to the invention, the reaction temperatures can be varied within a substantially wide range. The reaction is generally carried out at a temperature between about −80° C. and about +150° C., preferably between about −20° C. and about +100° C.

Process (d) according to the invention is generally carried out under atmospheric pressure but, if desired, can also be carried out under elevated or reduced pressure.

When carrying out process (d) according to the invention, in general 1 mole of an isothiazole derivative of the formula (VII) is reacted with 1 to 1.5 moles of a chloromethyl compound of the formula (VIII) in the presence of a diluent, such as dimethylformamide, and in the presence of an acid-binding agent, such as sodium hydride.

Suitable diluents for conducting process (e) according to the invention are all customary inert organic solvents. The following can preferably be used:

aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable catalysts for conducting process (e) according to the invention are all customary acid catalysts.

Preferred catalysts of this type are mineral acids, for example, hydrochloric acid, sulfuric acid, nitric acid, hydrobromic acid, sodium hydrogen sulfite etc.; organic acids, for example, formic acid, acetic acid, trifluoroacetic acid, propionic acid, methanesulfonic acid, benzenesulfonic acid, p-toluenesulfonic acid etc.; organic amine hydrochlorides, for example, pyridine hydrochloride, triethylamine hydrochloride etc.; amine sulfonates, for example, pyridine p-toluenesulfonate, triethylamine p-tolenesulfonate etc.

When carrying out process (e) according to the invention, the reaction temperatures can be varied within a substantially wide range. The reaction is generally carried out at a temperature between about −20° C. and about +200° C., preferably between about 20° C. and about 150° C.

Process (e) according to the invention is generally carried out under atmospheric pressure but, if desired, can also be carried out under elevated or reduced pressure.

When carrying out process (e) according to the invention, in general 1 mole of 3,4-dichloro-isothiazole-5-carboxamide of the formula (11) is reacted with 1 to 1.5 moles of a formyl compound of the formula (IX) and 1 to 1.5 moles of 1H-benzotriazole of the formula (X) in the presence of a diluent, such as toluene, and in the presence of an acid catalyst, such as p-toluenesulfonic acid monohydrate.

Suitable diluents for conducting process (f) according to the invention are all customary inert organic solvents and water. The following can preferably be used: water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable acid-binding agents for conducting process (f) according to the invention are all customary inorganic and organic bases. The following can preferably be used: Inorganic bases, such as, hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide etc.; inorganic alkali metal amides, for example, lithium amide, sodium amide, potassium amide etc.; organic bases, such as, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying out process (f) according to the invention, the reaction temperatures can be varied within a substantially wide range. The reaction is generally carried out at a temperature between about −80° C. and about +200° C., preferably between about −300° C. and about +100° C.

Process (f) according to the invention is generally carried out under atmospheric pressure but, if desired, can also be carried out under elevated or reduced pressure.

When carrying out process (f) according to the invention, in general 1 mole of 3,4-dichloro-isothiazole-5-carbonylchloride of the formula (XI) is reacted with a compound of the formula (XII) in the presence of a diluent, such as methylene chloride and in the presence of an acid-binding agent, such as triethylamine.

In a particular variant, process (f) according to the invention can also be carried out by preparing a compound of the formula (XII), in which

Y¹ represents a group of the formula

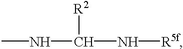

by the process described in J. Chem. Soc., Perkin Trans. 1, 2339-2344 (1988), and reacting same without prior isolation with 3,4-dichloro-isothiazole-5-carbonylchloride.

Suitable diluents for conducting process (g) according to the invetnion are all customary inert organic solvents and water. The following can preferably be used: Water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable acid-binding agents for conducting process (g) according to the invention are all customary inorganic and organic bases. The following can preferably be used: Inorganic bases, such as, hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide etc.; inorganic alkali metal amides, for example, lithium amide, sodium amide, potassium amide etc.; organic bases, such as, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying out process (g) according to the invention, the reaction temperatures can be varied within a substantially wide range. The reaction is generally carried out at a temperature between about −80° C. and about +150° C., preferably between about −20° C. and about +100° C.

Process (g) according to the invention is generally carried out under atmospheric pressure but, if desired, can also be carried out under elevated or reduced pressure.

When carrying out process (g) according to the invention, in general 1 mole of a 3,4-dichloro-isothiazole-5-carboxylic acid ester of the formula (XIII) is reacted with 1 to 1.5 moles of a compound of the formula (XIV) in the presence of a diluent, such as methanol.

Suitable diluents for conducting process (h) according to the invention are all customary inert organic solvents and water. The following can preferably be used: Water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for conducting process (h) according to the invention are all customary inorganic and organic bases. The following can preferably be used: Inorganic bases, such as, hydroxides, carbonates, bicarbonates, acetates etc. of alkali metals and alkaline earth metals, for example, sodium hydrogen carbonate, potassium hydrogen carbonate, sodium carbonate, potassium carbonate, lithium hydroxide, sodium hydroxide, potassium hydroxide, calcium hydroxide, sodium acetate etc.; organic bases, such as, tertiary amines, dialkylaminfoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.

Suitable catalysts for conducting process (h) according to the invention are all customary acid catalysts. Preferred catalysts of this type are mineral acids, for example, hydrochloric acid, sulfuric acid, nitric acid, hydrobromic acid, sodium hydrogen sulfite etc.; organic acids, for example, formic acid, acetic acid, trifluoroacetic acid, propionic acid, methanesulfonic acid, benzenesulfonic acid, p-toluenesulfonic acid etc.; organic amine hydrochlorides, for example, pyridine hydrochloride, triethylamine hydrochloride etc.; amine sulfonates, for example, pyridine p-toluenesulfonate, triethylamine p-toluenesulfonate etc.

When carrying our process (h) according to the invention, the reaction temperatures can be varied within a substantially wide range. The reaction is generally carried out at a temperature between about −20° C. and about +150° C., preferably between about 0° C. and about 120° C.

Process (h) according to the invention is generally carried out under atmospheric pressure but, if desired, can also be carried out under elevated or reduced pressure.

When carrying out process (h) according to the invention, in general 1 mole of 3,4-dichloro-isothiazole-5-carbohyrazide of the formula (XV) is reacted with 1 to 1.5 moles of a compound of the formula (XVI) in the presence of a diluent, such as ethanol, and in the presence of an acid-binding agent, such as sodium acetate.

Suitable diluents for conducting process (i) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable acid-binding agents for conducting process (i) according to the invention are all customary inorganic and organic bases. The following can preferably be used:

Hydrides of alkali metals and alkaline earth metals, for example, sodium hydride, lithium hydride etc.; inorganic alkali metal amides, for example, lithium amide, sodium amide, potassium amide etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying out process (i) according to the invention, the reaction temperatures can be varied within a substantially wide range. The reaction is generally carried out at a temperature between about −80° C. and about +150° C., preferably between about −20° C. and about +50° C.

Process (i) according to the invention is generally carried out under atmospheric pressure but, if desired, can also be carried out under elevated or reduced pressure.

When carrying out process (i) according to the invention, in general 1 mole of 3,4-dichloro-isothiazole-5-carbohydrazide of the formula (XV) is reacted with 1 to 1.2 moles of a compound of the formula (XVII) in the presence of a diluent, such as N,N-dimethylformamide, and in the presence of a acid-binding agent, such as sodium hydride.

Suitable diluents for conducting process (i) according to the invention are all customary inert organic solvents and water. The following can preferably be used: Water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable catalysts for conducting process (j) according to the invention are all customary acid catalysts. Preferred catalysts of this type are mineral acids, for example, hydrochloric acid, sulfuric acid, nitric acid, hydrobromic acid, sodium hydrogen sulfite etc.; organic acids, for example, formic acid, acetic acid, trifluoroacetic acid, propionic acid, methanesulfonic acid, benzenesulfonic acid, p-toluenesulfonic acid etc.; organic amine hydrochlorides, for example, pyridine hydrochloride, triethylamine hydrochloride etc.; amine sulfonates, for example, pyridine p-toluenesulfonate, triethylamine p-toluenesulfonate etc.

When carrying out process (j) according to the invention, the reaction temperatures can be varied within a substantially wide range. The reaction is generally carried out at a temperature between about −20° C. and about +150° C., preferably between about 0° C. and about 1° C.

Process (j) according to the invention is generally carried out under atmospheric pressure but, if desired, can also be carried out under elevated or reduced pressure.

When carrying out process (j) according to the invention, in general 1 mole of 3,4-dichloro-isothiazole-5-carbohydrazide of the formula (XV) is reacted with 1 to 1.2 moles of a compound of the formula (XVIII) in the presence of a diluent, such as ethanol.

Suitable diluents for conducting process (k) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

When carrying out process (k) according to the invention, the reaction temperatures can be varied within a substantially wide range. The reaction is generally carried out at a temperature between about −20° C. and about +150° C., preferably between about 0° C. and about 100° C.

Process (k) according to the invention is generally carried out under atmospheric pressure but, if desired, can also be carried out under elevated or reduced pressure.

When carrying out process (k) according to the invention, in general 1 mole of an isothiazolecarboxylic acid derivative of the formula (Ia) is reacted with 1 to 2 moles of an oxydizing agent, such as m-chloro-perbenzoic acid in the presence of a diluent, such as methylene chloride.

Suitable diluents for conducting process (I) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable catalysts for conducting process (l) according to the invetion are all customary acid catalysts.

Preferred catalysts of this type are mineral acids, for example, hydrochloric acid, sulfuric acid, nitric acid, hydrobromic acid, sodium hydrogen sulfite etc.; organic acids, for example, formic acid, acetic acid, trifluoroacetic acid, propionic acid, methanesulfonic acid, benzenesulfonic acid, p-toluenesulfonic acid etc.; organic amine hydrochlorides, for example, pyridine hydrochloride, triethylamine hydrochloride etc.; amine sulfonates, for example, pyridine p-toluenesulfonate, triethylamine p-toluenesulfonate etc.

When carrying out process (I) according to the invention, the reaction temperatures can be varied within a substantially wide range. The reaction is generally carried out at a temperature between about 0° C. and about 200° C., preferably between about 20° C. and about 150° C.

Process (l) according to the invention is generally carried out under atmospheric pressure but, if desired, can also be carried out under elevated or reduced pressure.

When carrying out process (l) according to the invention, in general 1 mole of 3,4-dichloro-isothiazole-5-carboxamide of the formula (II) is reacted with 1 to 100 moles of a compound of the formula (XIX).

Suitable diluents for conducting process (m) according to the invention are all customary inert organic solvents and water. The following can preferably be used: Water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitrites, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

When carrying out process (m) according to the invention, the reaction temperatures can be varied within a substantially wide range. The reaction is generally carried out at a temperature between about −20° C. and about +100° C., preferably between about 0° C. and about 50° C.

Process (m) according to the invention is generally carried out under atmospheric pressure but, if desired, can also be carried out under elevated or reduced pressure.

When carrying out process (m) according to the invention, in general 1 mole of an isothiazolecarboxylic acid derivative of the formula (Ib) is reacted with 1 to 2 moles of an oxidizing agent, such as trimethylamine N-oxide in the presence of osmium (VIII) oxide, and in the presence of a diluent, such as aqueous tetrahydrofuran (water:tetrahydrofuran=1:30).

Suitable diluents for conducting process (n) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitrites, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable catalysts for conducting process (n) according to the invention are all customary acid catalysts. Preferred catalysts of this type are mineral acids, for example, hydrochloric acid, sulfuric acid, nitric acid, hydrobromic acid, sodium hydrogen sulfite etc.; organic acids, for example, formic acid, acetic acid, trifluoroacetic acid, propionic acid, methanesulfonic acid, benzenesulfonic acid, p-toluenesulfonic acid etc.; organic amine hydrochlorides, for example, pyridine hydrochloride, triethylamine hydrochloride etc.; amine sulfonates, for example, pyridine p-toluenesulfonate, triethylamine p-toluenesulfonate etc.

When carrying out process (n) according to the invention, the reaction temperatures can be varied within a substantially wide range. The reaction is generally carried out at a temperature between about −20° C. and about +200° C., preferably between about 0° C. and about 150° C.

Process (n) according to the invention is generally carried out under atmospheric pressure but, if desired, can also be carried out under elevated or reduced pressure.

When carrying out process (n) according to the invention, in general 1 mole of an isothiazolecarboxylic acid derivative of the formula (Ic) is reacted with 1 to 2 moles of a compound of the formula (XX) in the presence of diluent and in the presence of a catalyst, such as p-toluenesulfonic acid.

Suitable diluents for conducting process (o) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.

Suitable acid-binding agents for conducting process (o) according to the invention are all customary inorganic and organic bases. The following can preferably be used: Hydrides of alkali metals and alkaline earth metals, for example, sodium hydride, lithium hydride etc.; inorganic alkali metal amides, for example, lithium amide, sodium amide, potassium amide etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying out process (o) according to the invention, the reaction temperatures can be varied within a substantially wide range. The reaction is generally carried out at a temperature between about −80° C. and about +100° C., preferably between about −20° C. and about +80° C.

Process (o) according to the invention is generally carried out under atmospheric pressure but, if desired, can also be carried out under elevated or reduced pressure.

When carrying out process (o) according to the invention, in general 1 mole of 3,4-dichloro-isothiazole-5-carboxamide of the formula (II) is reacted with 1 to 1.2 moles of a cyano compound of the formula (XXI) in the presence of a diluent, such as tetrahydrofuran, and in the presence of an acid-binding agent, such as sodium hydride.

Suitable diluents for conducting process (p) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, propionitrile, acrylonitrile etc.; alcohols, for example, methanol, ethanol, isopropanol, butanol, ethylene glycol etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for conducting process (p) according to the invention are all customary inorganic and organic bases. The following can preferably be used: Inorganic bases, such as hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide etc.; inorganic alkali metal amides, for example, lithium amide, sodium amide, potassium amide etc.; organic bases, such as, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-DBU, n-butyl lithium-TMEDA etc.

When carrying out process (p) according to the invention, the reaction temperatures can be varied within a substantially wide range. The reaction is generally carried out at a temperature between about −20° C. and about +200° C., preferably between about 0° C. and about 150° C.

Process (p) according to the invention is generally carried out under atmospheric pressure but, if desired, can also be carried out under elevated or reduced pressure.

When carrying out process (p) according to the invention, in general 1 mole of a 3,4-dichloro-isothiazole derivative of the formula (XXII) is reacted with 1 to 2 moles of a compound of the formula (XIII) in the presence of a diluent, such as toluene, and in the presence of 1 to 1.2 moles of an acid-binding agent, such as 4-dimethylaminopyridine.

Suitable diluents for conducting process (q) according to the invention are all customary inert organic solvents and water. The following can preferably be used: Water; aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for conducting process (q) according to the invention are all customary inorganic and organic bases. The following can preferably be used: Inorganic bases, such as, hydrides, hydroxides, carbonates, bicarbonates etc. of alkali metals and alkaline earth metals, for example, sodium hydride, lithium hydride, sodium hydrogen carbonate, potassium hydrogen carbonate, sodium carbonate, potassium carbonate, organic bases, such as, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-diazabicyclo-[5,4,0]undec-7-ene (DBU) etc.; organolithium compounds, for example, methyl lithium, n-butyl lithium, sec-butyl lithium, tert-butyl lithium, phenyl lithium, dimethyl copper lithium, lithium diisopropylamide, lithium cyclohexylsopropylamide, lithium dicyclohexylamide, n-butyl lithium-DABCO, n-butyl lithium-DBU, n-butyl lithium-TMEDA etc.

When carrying out process (q) according to the invention, the reaction temperatures can be varied within a substantially wide range. The reaction is generally carried out at a temperature between about −80° C. and about +150° C., preferably between about −10° C. and about +100° C.

Process (q) according to the invention is generally carried out under atmospheric pressure but, if desired, can also be carried out under elevated or reduced pressure.

When carrying out process (q) according to the invention, in general 1 mole of a 3,4-dichloro-isothiazole derivative of the formula (Id) is reacted with 1 to 1.2 moles of a chloro-substituted compound of the formula (XXIV) in the presence of a diluent, such as methylene chloride, and in the presence of an acid-binding agent, such as triethylamine.

Suitable diluents for conducting process (r) according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; bases, for example, pyridine etc.

Suitable acid-binding agents for conducting process (r) according to the invention are inorganic bases, such as hydrides, carbonates and bicarbonates of alkali metals and alkaline earth metals, for example, sodium hydride, lithium hydride, sodium carbonate etc.

Suitable catalysts for conducting process (r) according to the invention are tertiary amines, dialkylaminoanilines and pyridines, for example, triethylamine, N,N-dimethylaniline, N,N-diethylaniline, pyridine, 4-dimethylaminopyridine (DAMP) etc.

When carrying out process (r) according to the invention, the reaction temperatures can be varied within a substantially wide range. The reaction is generally carried out at a temperature between about −80° C. and about +150° C., preferably between about −10° C and about +100° C.

Process (r) according to the invention is generally carried out under atmospheric pressure but, if desired, can also be carried out under elevated or reduced pressure.

When carrying out process (r) according to the invention, in general 1 mole of a 3,4-dichloro-isothiazole derivative of the formula (Id) is reacted with 1 to 1.2 moles of an isocyanate of the formula (XXV) in the presence of a diluent, such as methylene chloride, and in the presence of a catalytic amount of a catalyst, such as pyridine.

The compounds of the formula (I) prepared by the above-mentioned processes can in each case be isolated from the reaction mixtures by customary procedures and can be purified by known methods, such as crystallization, chromatography etc.

The compounds according to the present invention exhibit a strong microbicidal activity. Thus, they can be used for combating undesired microorganisms, such as phytopathogenic fungi and bacteriae, in agriculture and horticulture. The compounds are suitable for the direct control of undesired microorganisms as well as for generating resistance in plants against attack by undesired plant pathogens.

Resistance-inducing substances in the present context are to be understood as those substances which are capable of stimulating the defence system of plants such that the treated plants, when subsequently inoculated with undesirable microorganisms, display substantial resistance to these microorganisms.

Undesirable microorganisms in the present case are to be understood as phytopathogenic fungi and bacteriae. The substances according to the invention can thus be employed to generate resistance in plants against attack by the harmful organisms mentioned within a certain period of time after the treatment. The period of time within which resistance is brought about in general extends from 1 to 10 days, preferably 1 to 7 days, after treatment of the plants with the active compounds.

Generally, the compounds according to the invention can be used as fungicides for combating phytopathogenic fungi, such as Plasmodiophoromycetes, Oomycetes, Chytridiomycetes, Zygomycetes, Ascomycetes, Basidiomycetes and Deuteromycetes, and can also be used as bactericides for combating bacteriae, such as Pseudomonadaceae, Rhizobiaceae, Enterobacteriaceae, Corynebacteriaceae and Streptomycetaceae.

The compounds according to the present invention are particularly suitable for causing resistance against infection of plants by plant pathogens, such as Pyricularia oryzae, Phythophthora infestans etc.

The good toleration, by plants, of the active compounds, at the concentrations required for combating plants diseases, permits treatment of above-ground parts of plants, of vegetative propagation stock and seeds, and of the soil.

The compounds according to the present invention have a low toxicity against warm-blooded animals and therefore can be used safely.

The active compounds can be converted into the customary formulations, such as solutions, emulsions, wettable powders, suspensions, powders, foams, pastes, granules, tablets, aerosols, natural and synthetic materials impregnated with active compound, very fine capsules in polymeric substances, coating compositions for use on seed, and formulations used with burning equipment, such as fumigating cartridges, fumigating cans and fumigating coils, as well as ULV cold mist and warm mist formulations.

These formulations may be produced in known manner, for example by mixing the active compounds with extenders, that is to say liquid or liquefied gaseous or solid diluents or carriers, optionally with the use of surface-active agents, that is to say emulsifying agents and/or dispersing agents and/or foam-forming agents. In the case of the use of water as an extender, organic solvents can, for example, also be used as auxiliary solvents.

As liquid solvents diluents or carriers, there are suitable in the main, aromatic hydrocarbons such as xylene, toluene or alkyl naphthalenes, chlorinated aromatic or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl-isobutyl ketone or cyclohexanone, or strongly polar solvents, such as dimethylformamide and dimethyl-sulphoxide, as well as water.

By liquefied gaseous diluents or carriers are meant liquids which would be gaseous at normal temperature and under normal pressure, for example aerosol propellants, such as halogenated hydrocarbons as well as butane, propane, nitrogen and carbon dioxide.

As solid carriers there may be used ground natural minerals, such as kaolings, clays, talc, chalk, quartz, attapulgite, montmorillonite or diatomaceous earth, and ground synthetic minerals, such as highly-dispersed silicic acid, alumina and silicates. As solid carriers for granules there may be used crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks.

As emulsifying and/or foam-forming agents there may be used non-ionic and anionic emulsifiers, such as polyoxyethylene-fatty acid esters, polyoxyethylene-fatty alcohol ethers, for example alkylaryl polyglycol ethers, alkyl sulphonates, alkyl sulphates, aryl sulphonates as well as albumin hydrolysis products.

Dispersing agents include, for example, lignin sulphite waste liquors and methylcellulose.

Adhesives such as carboxymethylcellulose and natural and synthetic polymers in the form of powders, granules or latices, such as gum arabic, polyvinyl alcohol and polyvinyl acetate, can be used in the formulation.

It is possible to use colorants such as inorganic pigments, for example iron oxide, titanium oxide and Prussian Blue, and organic dyestuffs, such as alizarin dyestuffs, azo dyestuffs or metal phthalocyanine dyestuffs, and trace nutrients, such as salts of iron, manganese, boron, copper, cobalt, molybdenum and zinc.

The formulations in general contain from 0.1 to 95 percent by weight of active compound, preferably from 0.5 to 90 percent by weight.

The active compounds according to the invention can be present in the formulations or in the various use forms as a mixture with other known active compounds, such as fungicides, bactericides, insecticides, acaricides, nematicides, herbicides, bird repellents, growth factors, plant nutrients and agents for improving soil structure.

In many cases, synergistic effects are achieved, i.e. the activity of the mixture exceeds the activity of the individual components.

Examples of co-components in mixtures are the following compounds:

Fungicides:

aldimorph, ampropylfos, ampropylfos potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamide,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorpb, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, fuircarbonil, furconazole, furconazolecis, funrnecyclox, fenhexamide,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione, iprovalicarb,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclarn, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur,

quinconazole, quintozene (PCNB), quinoxyfen,

sulphur and sulphur preparations, spiroxamine,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole, trifloxystrobin,

uniconazole,

validamycin A, vinclozolin, viniconazole,

zarilamide, zineb, ziram and also

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone O-(phenylmethyl)-oxime,

1-(2-methyl-1-naphthalenyl)-1H-pyrrol-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′,6′-dibromo-2-methyl-4′-trifluoromethoxy-4′-trifluoro-methyl-1,3-thiazole-5-carboxanilide,

2,6-dichloro-5-(methylthio)-4-pyrimidinyl thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-p-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrol-2,5-dione,

3,5-dichloro-N-[cyano[(1-methyl-2-propinyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-hydroxyquinoline sulphate,

9H-xanthene-2-[(phenylamino)-carbonyl]-9-carboxylic hydrazide,

b is-(1-methylethyl)-3-methyl-4-[(3-methylbenzoyl)-oxy] 2,5-thiophenedicarboxylate,

c is-1-(4-chlorophenyl)-2-(1H-1,2,4-triazol-1-yl)-cycloheptanol,

c is-4-[3-[4-(1,1-dimethylpropyl)-phenyl-2-methylpropyl]-2,6-dimethylmorpholinehydrochloride,

ethyl [(4-chlorophenyl)-azo]-cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)-acetaride,

N-(6-methoxy)-3-pyridinyl)-cyclopropanecarboxamide,

N-[2,2,2-trichloro-1-[(chloroacetyl)-amino]-ethyl]-benzamide,

N-[3-chloro-4,5-bis(2-propinyloxy)-phenyl]-N′-methoxy-methanimidamide,

N-formyl-N-hydroxy-DL-alanine-sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran]-3′-one,

Bactericides:

bronopol, dichlorophen, nitrapyrin, nickel dimethyldithiocarbamate, kasugarnycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, cis-resmethrin, cispennethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, diflubenzuron, dimethoat, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimphos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, isazophos, isofenphos, isoxathion, ivermectin,

lambda-cyhalothrin, lufenuron,

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron, nuclear polyhedrosis viruses,

omethoat, oxamyl, oxydemethon M,

Paecilomyces filmosoroseus, parathion A, parathion M, permethrin, phenthoat, phorat, phosalone, phosmet, phosphamidon, phoxim, pirimicarb, pirimiphos A, pirimiphos M, profenofos, promecarb, propoxur, prothiofos, prothoat, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb, thiacloprid,

vamidothion, vaniliprole, Verticillium lecanii,

YI 5302,

zeta-cypermethrin, zolaprofos,

(1R-cis)-[5-(phenylmethyl)-3-furanyl]-methyl-3-[(dihydro-2-oxo-3(2H)furanylidene)-methyl] 2,2-dimethylcyclopropanecarboxylate,

(3-phenoxyphenyl)-methyl 2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate.

The active compounds can be used as such or in the form of their formulations or the use forms prepared therefrom by further dilution, such as ready-to-use solutions, emulsions, suspensions, powders, tablets, pastes, microcapsules and granules. They are used in the customary manner, for example by watering, immersion, spraying, atomising, misting, vaporizing, injecting, forming a slurry, brushing on, dusting, scattering, dry dressing, moist dressing, wet dressing, slurry dressing or encrusting.

In the treatment of parts of plants, the active compounds concentration in the use forms can be varied within a substantial range. They are, in general, from 1 to 0.0001% by weight, preferably from 0.5 and 0.001%.

For the treatment of seed, amounts of active compound of 0.001 to 50 g, especially 0.01 to 10 g, are generally employed per kilogram of seed.

For the treatment of soil, active compound concentrations, at the point of action, of 0.00001 to 0.1% by weight, especially of 0.0001 to 0.02%, are generally employed.

As already mentioned above, all plants and parts of plants can be treated according to the invention. In a preferred embodiment naturally occurring plant species and plant varieties or those obtained by conventional biological breeding methods, such as crossbreeding or protoplast fusion as well as parts of such plants are treated. In an additional preferred embodiment transgenic plants and plant varieties which have been obtained by genetic engineering methods, possibly in combination with conventional methods (genetically modified organisms) and parts of such plants are treated. The term “parts” or “parts of plants” or “plant parts” is explained above.

According to the invention plants of the plant varieties commercially available or used at any particular time are very preferably treated. Plant varieties are understood to be plants with specific properties (“traits”) which have been obtained both by conventional breeding, by mutagenesis or by recombinant DNA techniques. They can be varieties, biotypes or genotypes.

Depending on the species or varieties of plants, their location and growth conditions (the types of soil, climate, vegetation period and feed concerned), superadditive (“synergistic”) effects can occur as a result of the treatment according to the invention. Effects such as for example reduced application rates and/or broadening of the activity spectra and/or increased activity of the compounds and compositions usable according to the invention, improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops are possible, which are greater than those actually expected.

Preferred transgenic plants or plant varieties (obtained by genetic engineering) to be treated according to the invention include all plants which as a result of the genetic modification concerned have received genetic material which provides them with particularly advantageous valuable properties (“traits”). Examples of such properties are improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops. Additional and particularly noteworthy examples of such properties are increased resistance of the plants to animal and microbial pests, such as to insects, mites, phytopathogenic fungi, bacteria and/or viruses as well as increased tolerance by the plants of certain herbicidal active compounds. Examples which may be mentioned of transgenic plants are the important crop plants such as cereals (wheat and rice), corn, soybeans, potatoes, cotton, rape and fruit plants (producing apples, pears, citrus fruits and grapes), the crop plants corn, soybeans, potatoes, cotton and rape being particularly noteworthy. Particularly significant properties (“traits”) are increased resistance of the plants to insects due to the toxins forming in the plants, and in particular those which are produced in the plants (hereinafter referred to as “Bt plants”) by the genetic material obtained from Bacillus Thuringiensis (e.g. by the genes CryIA(a), CryIA(b), CryIA(c), CryIIA, CryIIIB2, Cry9c Cry2Ab, Cry3Bb and CryIF and combinations thereof). Particularly significant properties (“traits”) are the increased resistance of plants to fungi, bacteria and viruses due to systemically acquired resistance (SAR), systemin, phytoalexins, elicitors and resistance genes and correspondingly expressed proteins and toxins. Particulary significant properties (“traits”) are also increased tolerance by the plants of certain herbicidal active compounds, such as for example imidazolinones, sulphonylureas, glyphosate or phosphinotricine (e.g. the “PAT” gene). The corresponding genes imparting the required properties (“traits”) can also occur in the transgenic plants in combination with each other. Examples which may be mentioned of “Bt plants” are varieties of corn, cotton, soybeans and potatoes which are sold under the trade names YIELD GARD° (e.g. corn, cotton, soybeans), KnockOut® (e.g. corn), StarLink® (e.g. corn), Bollgard® (cotton), Nucotn® (cotton) and NewLeaf® (potatoes). Examples which may be mentioned of herbicide-tolerant plants are varieties of corn, cotton and soybeans which are sold under the trade names Roundup Ready® (tolerance of glyphosate, e.g. corn, cotton, soybeans), Liberty Link® (tolerance of phosphinotricine, e.g. rape), IMI® (tolerance of imidazolinones) and STS® (tolerance of sulphonylureas, e.g. corn). Herbicide-resistant plants (bred for herbicide tolerance in the conventional manner) which may be mentioned are also the varieties (e.g. corn) sold under the name Clearfield®. The above statements do of course also apply to any plant varieties which may be developed in the future or launched onto the market in the future and which have the genetic properties (“traits”) described above or developed in the future.

According to the invention the abovementioned plants can be particularly advantageously treated with the compounds of the general formula I or the active compound mixtures according to the invention. The preferred ranges mentioned above for the active compounds or mixtures also apply to the treatment of these plants. Particularly advantageous is the treatment of plants with the compounds or mixtures specifically listed in the present text.

Then the present invention will be described more specifically by the following examples. However, the present invention should not be restricted to them in any way.

### EXAMPLES

### Synthesis Example 1

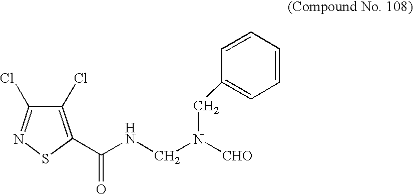

Process (a):

To a suspension of 3,4-dichloro-5-isothiazolecarboxamide (2.0 g) and N-benzyl-N-hydroxymethylformamide (1.7 g) in acetic acid (50 ml) sulfuric acid (2.2 g) was added and the mixture was stirred at room temperature for 16 hours. The reaction mixture was poured into water and extracted with methylene chloride. The organic layer was successively washed with saturated aqueous sodium hydrogen carbonate solution, water and then dried over anhydrous magnesium sulfate. The residue, obtained by distilling off the solvent under reduced pressure, was purified by silica gel column chromatography (eluant: hexane/ethyl acetate=2/3) to obtain N-[(benzylformamido)-methyl]-3,4-dichloro-5-isothiazolecarboxamide (1.6 g).

mp 102-103° C.

### Synthesis Example 2

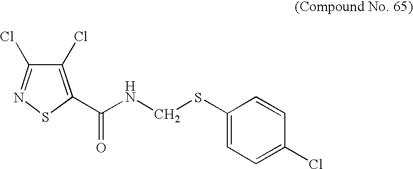

Process (b):

To a suspension of 60% sodium hydride (0.16 g) in tetrahydrofuran (50 ml) 4-chlorothiophenol (0.53 g) was added and the mixture was stirred for 15 minutes. N-Chloromethyl-3,4-dichloro-5-isothiazolecarboxamide (0.90 g) was then added and the mixture was stirred at room temperature for 16 hours. The reaction mixture was poured into water and extracted with methylene chloride. The organic layer was successively washed with diluted hydrochloric acid and a saturated solution of sodium chloride in water, and then it was dried over with anhydrous magnesium sulfate. The residue, obtained by distilling off the solvent under reduced pressure, was purified by silica gel column chromatography (eluant: chloroform/ethyl acetate=4/1) to obtain N-(4-chlorophenylthiomethyl)-3,4-dichloro-5-isothiazolecarboxamide (0.42 g).

mp 106-107° C.

### Synthesis Example 3

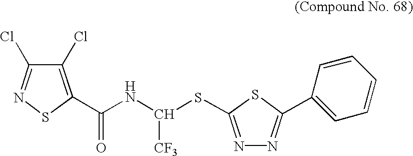

Process (b):

To a solution of N-(2,2,2-trifluoro-1-hydroxyethyl)-3,4-dichloro-5-isothiazolecarboxamide (1.00 g) in thionyl chloride (10 ml) a drop of N,N-dimethylformamide was added. The mixture was refluxed for 4 hours by heating and then thionyl chloride was distilled off under reduced pressure. The residue was added to a solution, obtained by adding 5-phenyl-1,3,4-thiadiazole-2-thiol (0.62 g) to a suspension of 60% sodium hydride (0.14 g) in N,N-dimethylformamide (30 ml) and stirring for 15 minutes, and stirred at room temperature for 16 hours. After distilling off the solvent under reduced pressure, water was added to residue and the resulting mixture was extracted with ethyl acetate. The organic layer was washed with a saturated solution of sodium chloride in water and then dried over anhydrous magnesium sulfate. The residue, obtained by distilling off the solvent under reduced pressure, was purified by silica gel column chromatography (eluant: hexane/ethyl acetate=2/1) to obtain N-[2,2,2-trifluoro-1-(5-phenyl-1,3,4-thiadiazol-2-ylthio)-ethyl]-3,4-dichloro-5-isothiazolecarboxamide (0.09 g).

mp 124-127° C.

### Synthesis Example 4

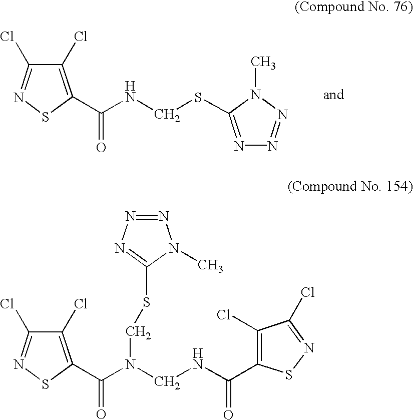

Process (b):

To a suspension of 60% sodium hydride (0.18 g) in N,N-dimethylformamide (30 ml) 5-mercapto-1-methyltetrazole (0.47 g) was added and the mixture was stirred for 15 minutes. Then N-chloromethyl-3,4-dichloro-5-isothiazolecarboxamide (1.00 g) was added and the mixture was stirred at room temperature for 16 hours. The reaction solution was poured into water and extracted with ethyl acetate. The organic layer was washed with a saturated solution of sodium chloride, water and dried over an, hydrous magnesium sulfate. The residue, obtained by distilling off the solvent under reduced pressure, was purified by silica gel column chromatography (eluant: hexane/ethyl acetate=2/1) to obtain N-(1-methyl-5-tetrazolethiomethyl)-3,4-dichloro-5-isothiazolecarboxamide (0.40 g, mp 176-177° C.) and N-[(3,4-dichloro-5-isothiazolecarboxamido)-methyl]-N-(1-methyl-5-tetrazolethiomethyl)-3,4-dichloro-5-isothiazolecarboxamide (0.26 g, mp 185-188° C.).

### Synthesis Example 5

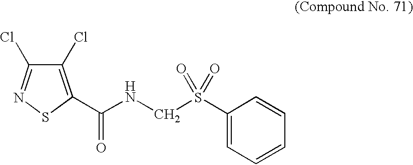

Process (b):

To a suspension of N-chloromethyl-3,4-dichloro-5-isothiazolecarboxamide (1.00 g) and sodium benzenesulfinate dihydrate (0.90 g) in dimethoxyethane, tetrabutylammonium bromide (0.05 g) was added, and the mixture was refluxed for 6 hours by heating. The reaction mixture was poured into water and the deposited crystals were filtered off to obtain N-phenylsulfonylmethyl-3,4-dichloro-5-isothiazolecarboxamide (1.00 g).

mp 175-176° C.

### Synthesis Example 6

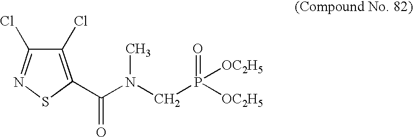

Process (c):

A mixed solution of N-chloromethyl-3,4-dichloro-5-isothiazolecarboxamide (1.2 g) and triethyl phosphite (10 ml) was stirred at 80° C. for 4 hours. The solvent was distilled off under reduced pressure and the residue was purified by silica gel column chromatography (hexane/ethyl acetate=1/1 to 1/4 gradient elution) to obtain diethyl N-methyl-N-(3,4-dichloro-5-isothiazolecarbonyl)-aminomethylphosphonate (1.1 g).

n_{D}²⁰ 1.5292

### Synthesis Example 7

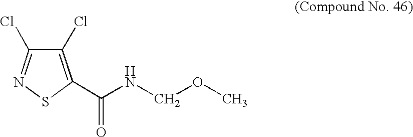

Process (d):

To a suspension of 60% sodium hydride (0.45 g) in tetrahydrofuran (100 ml) 3,4-dichloro-5-isothiazolecarboxamide (2.00 g) was added under ice cooling and the mixture was stirred for 15 minutes. Chloromethyl methyl ether (0.82 g) was added and the resulting mixure was stirred at room temperature for 16 hours. The reaction mixture was poured into water and extracted with methylene chloride. The organic layer was washed with diluted hydrochloric acid and then with a saturated solution of sodium chloride in water, and then dried over anhydrous magnesium sulfate. The residue, obtained by distilling off the solvent under reduced pressure, was purified by silica gel column chromatography (eluant: chloroform/ethyl acetate=4/1) to obtain N-methoxymethyl-3,4-dichloro-5-isothiazolecarboxamide (1.35 g).

mp 79-87° C.

### Synthesis Example 8

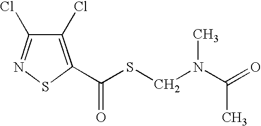

Process (d):

3,4-Dichloro-5-isothiazolecarbothioic O-acid (1.0 g) was added to a suspension of 60% sodium hydride (0.2 g) in tetrahydrofuran (50 ml) and the mixture was stirred for 15 minutes. N-Chloromethyl-N-methylacetamide (0.6 g) was then added and the resulting mixture was stirred for 16 hours. The reaction mixture was poured into water and extracted with methylene chloride. The organic layer was washed with a saturated solution of sodium chloride in water, and then dried over anhydrous magnesium sulfate. The solvent was distilled off under reduced pressure and the residue was purified by silica gel column chromatography (eluant: hexane/ethyl acetate=2/1) to obtain N-methylacetamidomethyl (3,4-dichloro-5-isothiazolecarbothioate (0.7 g).

n_{D}²⁰ 1.6012.

### Synthesis Example 9

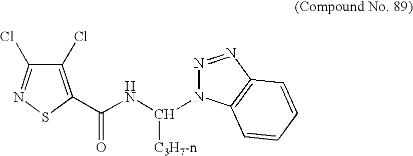

Process (e):

To a solution of 3,4-dichloro-5-isothiazolecarboxamide (1.0 g), butyraldehyde (0.4 g) and benzotriazole (0.6 g) in toluene a catalytic amount of p-toluenesulfonic acid monohydrate was added the mixture was refluxed for 5 hours by heating, eliminating the generated water. The residue, obtained by distilling off the solvent under reduced pressure, was purified by silica gel column chromatography (eluant: chloroform) to obtain N-(1-benzotriazol-1-yl)butyl)-3,4-dichloro-5-isothiazolecarboxamide (1. 1 g).

n_{D}²⁰ 1.5759.

### Synthesis Example 10

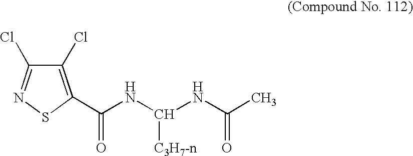

Process (f):

To a solution of acetamide (3.0 g), butyraldehyde (3.6 g) and benzotriazole (6.0 g) in toluene a catalytic amount of p-toluenesulfonic acid monohydrate was added and the mixture was refluxed for 5 hours by heating, eliminating the generated water. The residue, obtained by distilling off the solvent under reduced pressure, was purified by silica gel column chromatography (eluant: hexane/ethyl acetate=2/1) to obtain N-(1-benzotriazol-1-ylbutyl)-acetamide (4.2 g). The obtained solution of N-(1-benzotriazol-1-ylbutyl)-acetamide (4.2 g) and potassium carbonate (10.0 g) in methanol (50 ml) was saturated with ammonia gas under ice cooling and stirred at room temperature for 16 hours. The residue, obtained by filtering off the solid and distilling off the solvent under reduced pressure, (1.6 g) was dissolved in methylene chloride (10 ml). Triethylamine (2 ml) and 3,4-dichloro-5-isothiazolecarbonyl chloride (2.0 g) were then added at room temperature. After the reaction solution was washed with water and dried over anhydrous magnesium sulfate, the residue, obtained by distilling off the solvent under reduced pressure, was purified by silica gel column chromatography (eluant: hexane/ethyl acetate=1/1) to obtain N-[1-(acetamido)butyl]-3,4-dichloro-5-isothiazolecarboxamide (0.5 g).

mp 162-165° C.

### Synthesis Example 11

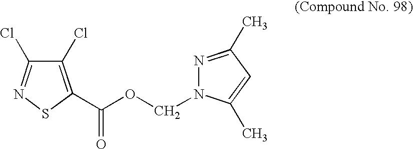

Process (f):

To a solution of 3,5-dimethyl-1-pyrazolylmethanol (0.58 g) and triethylamine (0.47 g) in methylene chloride 3,4-dichloro-5-isothiazolecarbonyl chloride (1.00 g) was added and the mixture was stirred at room temperature for 16 hours. The reaction mixture was poured into water and extracted with methylene chloride. The organic layer was washed with water and dried over anhydrous magnesium sulfate. The residue, obtained by distilling off the solvent under reduced pressure, was purified by silica gel column chromatography (eluant: ethyl acetate/hexane=1/2) to obtain 3,5-dimethylpyrazol-1-ylmethyl 3,4-dichloro-5-isothiazolecarboxylate (1.05 g).

mp 80-81° C.

### Synthesis Example 12

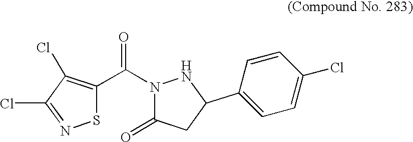

Process (h):

To a solution of 5-(4-chlorophenyl)-pyrazolidin-3-one (1.1 g) and 60% sodium hydride (0.2 g) in N,N-dimethylformamide (20 ml), after stirring for 1 hour, a solution of 3,4-dichloroisothiazol-5-ylcarbonyl chloride (1.00 g) in tetrahydrofuran (10 ml) was added at 0° C. and the mixture was stirred at 70° C. for 5 hours. After the solvent was distilled off under reduced pressure, the residue was purified by silica gel column chromatography (eluant: methylene chloride/ethanol=99/1) to obtain 2-(3,4-dichloro-3-isothiazolecarbonyl)-5-(4-chlorophenyl)-pyrazolidin-3-one (0.4 g).

mp 164-167° C.

### Synthesis Example 13

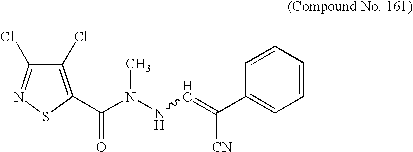

Process (f):

To a solution of N′-(2-cyano-2-phenylvinyl)-N-methylhydrazine (1.0 g) and triethylamine (0.6 g) in methylene chloride (20 ml), 3,4-dichloroisothiazol-5-ylcarbonyl chloride (1.1 g) was added and the mixture was stirred at room temperature for 4 hours. The solvent was distilled off under reduced pressure and the residue was purified by silica gel column chromatography (eluant: chloroform) to obtain N′-(2-cyano-2-phenylvinyl)-N-methyl-3,4-dichloro-5-isothiazolecarbohydrazide (0.6 g).

mp 130-139° C.

### Synthesis Example 14

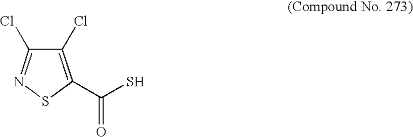

Process (f):

To a solution of potassium hydroxide (6.1 g) in ethanol (50 ml), saturated with hydrogen sulfide at 0° C., 3,4-dichloro-5-isothiazolecarbonyl chloride (10.0 g) was added at a temperature lower than 15° C. within more than 1 hour and the mixture was stirred for further 2 hours. The deposit was filtered off and the residue, obtained by distilling off the solvent under reduced pressure, was dissolved in cold water and washed with benzene. The aqueous solution was acidified with concentrated hydrochloric acid, extracted with ether, dried over anhydrous magnesium sulfate and the solvent was distilled off under reduced pressure to obtain 3,4-dichloro-5-isothiazolecarbothioic O-acid (8.1 g)

mp 79-80° C.

### Synthesis Example 15

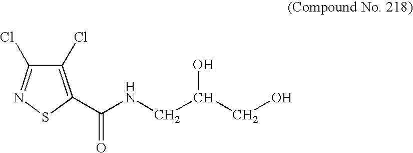

Process (g):

To a solution of 3-amino-1,2-propanediol (3.2 g) in methanol, methyl 3,4-dichloro-5-isothiazolecarboxylate (6.4 g) was added under ice cooling and the mixture was stirred at room temperature for 18 hours. Ethyl acetate was added to the residue, obtained by distilling off methanol under reduced pressure, and the organic layer was successively washed with diluted hydrochloric acid, a saturated solution of sodium chloride in water, saturated aqueous sodium hydrogen carbonate solution and then with a solution of sodium chloride in water. After the organic layer was dried over anhydrous magnesium sulfate, the solvent was distilled off under reduced pressure to obtain N-(2,3-dihydroxypropyl)-3,4-dichloro-5-isothiazolecarboxamide (6.6 g).

mp 68-72° C.

### Synthesis Example 16

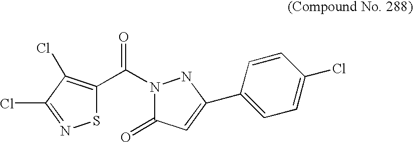

Process (h):

To a solution of diethyl (4-chlorobenzylidene)-malonate (1.33 g) and 3,4-dichloro-5-isothiazolecarbobydrazide (1.00 g) in ethyl acetate (30 ml), sodium acetate (0.39 g) was added and the mixture was refluxed for 6 hours by heating. The crystals, deposited upon adding water to the reaction mixture, were filtered off and washed with ethyl acetate to obtain 2-(3,4-dichloro-5-isothiazolecarbonyl)-5-(4-chlorophenyl)pyrazol-3-one (1.37 g).

mp higher than 250° C.

### Synthesis Example 17

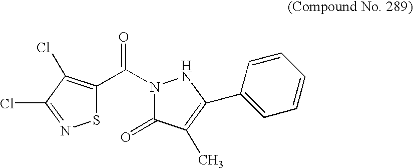

Process (i):

After adding 60% sodium hydride (0.2 g) in N,N-dimethylformamide to a solution of 3,4-dichloro-5-isothiazolecarbohydrazide (1.00 g) in N,N-dimethylformamide and stirring for 10 minutes, 2-methyl-3-phenyl-2-propenoyl chloride (0.9 g) was added under ice cooling and the mixture was stirred for 16 hours. The crystals, deposited upon adding water and ethyl acetate to the reaction mixture, were filtered off and purified by silica gel column chromatography (chloroform to chloroform/ethanol=98/2 gradient elution) to obtain 2-(3,4-dichloro-5-isothiazolecarbonyl)-4-methyl-5-phenylpyrazol-3-one (0.4 g).

mp 190-191° C.

### Synthesis Example 18

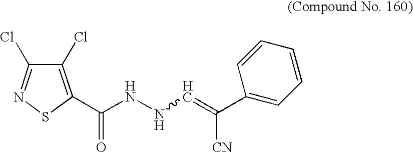

Process (j):

To a solution of 3,4-dichloro-5-isothiazolecarbohydrazide (0.6 g) and 2-formyl-2-phenylacetonitrile (0.4 g) in ethanol (40 ml) a catalytic amount of acetic acid was added and the mixture was refluxed for 3 hours by heating. After natural cooling, the deposited crystals were filtered off and washed with ethanol to obtain N′-(2-cyano-2-phenylvinyl)-3,4-dichloro-5-isothiazolecarbohydrazide (0.9 g).

mp 212-214° C.

### Synthesis Example 19

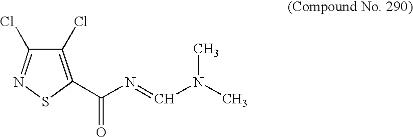

Process (l):

A mixed solution of 3,4-dichloro-5-isothiazolecarboxamide (1.0 g) and N,N-dimethylformamide dimethylacetal (5 ml) was stirred at 100° C. for 6 hours. The deposit was washed with water to obtain N-dimethylaminomethylidene-3,4-dichloro-5-iso thiazolecarboxamide (1. 1 g).

mp 196-197° C.

### Synthesis Example 20

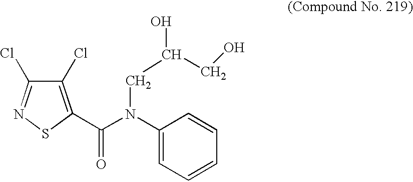

Process (m):

To a solution of N-allyl-N-phenyl-3,4-dichloro-5-isothiazolecarboxamide (0.94 g) and trimethylamine N-oxide dihydrate (0.45 g) in water-tetrahydrofuran (1 ml/30 ml) a catalytic amount of osmium (VIII) oxide was added and the resulting mixture was stirred at room temperature for 18 hours. An aqueous solution of sodium thiosulfate was added to the reaction mixture, which then was extracted with ethyl acetate, washed with a saturated solution of sodium chloride in water and then dried over anhydrous magnesium sulfate. The solvent was distilled off under reduced pressure and the residue was purified by silica gel column chromatography (methylene chloride to ethyl acetate gradient elution) to obtain N-(2,3-dihydroxypropyl)-N-phenyl-3,4-dichloro-5-isothiazolecarboxamide (1.0 g).

n_{D}²⁰ 1.5962

### Synthesis Example 21

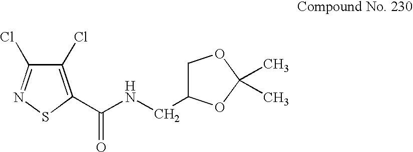

Process (n):

To a solution of N-(2,3-dihydroxypropyl)-3,4-dichloro-5-isothiazolecarboxamide (1.4 g) and acetone dimethylacetal (0.7 g) in 1,2-dichloroethane (30 ml) a catalytic amount of p-toluenesulfonic acid monohydrate was added and the mixture was refluxed for 2 hours by heating. The solvent was distilled off under reduced pressure and the residue was purified by silica gel column chromatography (eluant: methylene chloride/ethanol=96/4) to obtain N-[(2,2-dimethyl-1,3-dioxolan-4-yl)methyl]-3,4-dichloro-5-isothiazolecarboxamide (0.9 g).

mp 132-133° C.

### Synthesis Example 22

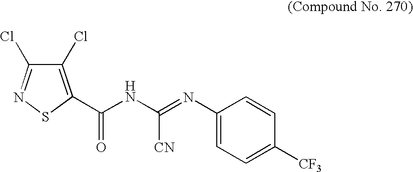

Process (o):

3,4-Dichloro-5-isothiazolecarboxamide (0.8 g) was added to a suspension of 60% sodium hydride (0.2 g) in tetrahydrofuran (30 ml) at 0° C. and the mixture was stirred for 30 minutes. 2-Chloro-2-(trifluoromethylphenyl)imino-acetonitrile (1.0 g) was then added at 0° C., and the mixture was stirred at room temperature for 16 hours.

The reaction mixture was poured into a mixture of ice and diluted hydrochloric acid, extracted with methylene chloride, and dried over anhydrous magnesium sulfate.

The solvent was distilled off under reduced pressure and the residue was purified by silica gel column chromatography (eluant: chloroform/ethyl acetate=98/2) to obtain N-[cyano-(4-trifluoromethylphenylimino)-methyl]-3,4-dichloro-5-isothiazolecarboxamide (0.6 g).

mp 151-153° C.

### Synthesis Example 23

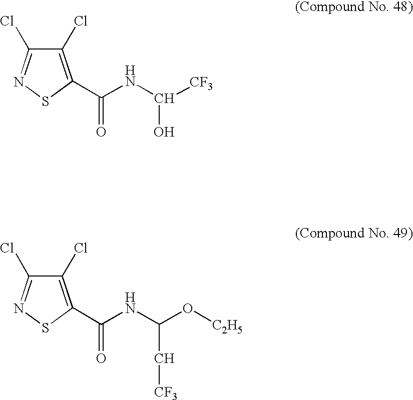

Process (p):

To a solution of 3,4-dichloro-5-isothiazolecarboxamide (2.00 g) and trifluoroacetaldehyde hemiethylacetal (1.63 g) in toluene, 4-dimethylaminopyridine (1.24 g) was added and the mixture was refluxed for 3 hours by heating. The reaction mixture was poured into water and extracted with ethyl acetate. The organic layer was washed with a saturated solution of sodium chloride in water and dried over anhydrous magnesium sulfate. The residue, obtained by distilling off the solvent under reduced pressure, was purified by silica gel column chromatography (eluant: ethyl acetate/hexane=1/2) to obtain N-(2,2,2-trifluoro-1-hydroxyethyl)-3,4-dichloro-5-isothiazolecarboxamide (0.50 g, mp 90-93° C.) and N-(2,2,2-trifluoro-1-ethoxyethyl)-3,4-dichloro-5-isothiazolecarboxamide (1.58 g, mp 99-101° C.).

### Synthesis Example 24

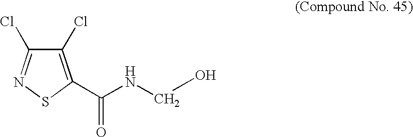

Process (p):

To a solution of 3,4-dichloro-5-isothiazolecarboxamide (10.0 g) and paraformaldehyde (1.80 g) in acetonitrile (300 ml), potassium carbonate (8.0 g) was added and the resulting mixture was stirred at room temperature for 5 hours. The reaction mixture was poured into ice water and the deposited crystals were filtered off, washed with water and dried to obtain N-hydroxymethyl-3,4-dichloro-5-isothiazolecarboxamide (6.3 g).

mp 90-93° C.

### Synthesis Example 25

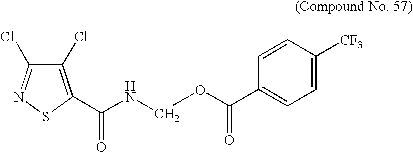

Process (q):

A solution of N-hydroxymethyl-3,4-dichloro-5-isothiazolecarboxamide (1.0 g) and triethylamine (0.5 g) in methylene chloride was cooled to 0° C. 4-Trifluoromethylbenzoyl chloride (0.9 g) was added and the mixture was stirred for a day. After washing the reaction mixture with water, the organic layer was dried over anhydrous magnesium sulfate. The residue, obtained by distilling off the solvent, was purified by silica gel column chromatography (eluant: methylene chloride) to obtain N-(4-trifluoromethylbenzoyloxy)-methyl-3,4-dichloro-5-isothiazolecarboxamide (1.2 g).

mp 120-121° C.

### Synthesis Example 26

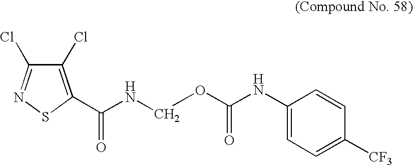

Process (r):

To a solution of N-hydroxymethyl-3,4-dichloro-5-isothiazolecarboxamide (1.0 g) in methylene chloride (50 ml), 3 drops of pyridine and 4-trifluoromethylphenyl isocyanate (0.8 g) were added and the mixture was stirred at 0° C. for a day. After washing the reaction solution with water, the organic layer was dried over anhydrous magnesium sulfate. The residue, obtained by distilling off the solvent, was purified by silica gel column chromatography (eluant: ethyl acetate:hexane=1:3) to obtain N-[N-(4-trifluoromethylphenyl)-carbamoyloxymethyl]-3,4-dichloro-5-isothiazolecarboxamide (0.7 g).

mp 117-119° C.

The compounds obtained in a similar manner as the above-mentioned Synthesis Examples 1-26 are shown, together with the compounds synthesized in Synthesis Examples 1-26, in the following Tables 1-7.

TABLE 1

| | |
|---|---|
| | (I) |
| 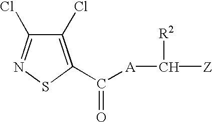 | |

| | | | | Melting point |
|---|---|---|---|---|
| Compound | | | | (mp)(° C.) or |
| No. | A | R² | Z | n₂₀^{D} |
| 1. | NH | H | 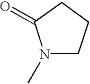 | 168-170 |
| 2. | NCH₃ | H | 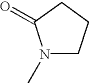 | |
| 3. | NH | H | 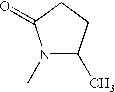 | |
| 4. | NH | H | 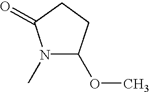 | |
| 5. | NH | H | 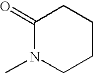 | 134 |
| 6. | NCH₃ | H | 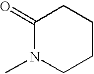 | |
| 7. | NH | H | 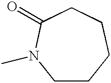 | 163-164 |
| 8. | NCH₃ | H | 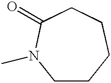 | |
| 9. | NH | H | 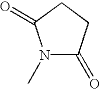 | |
| 10. | NCH₃ | H | 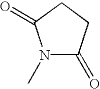 | |
| 11. | NH | H | 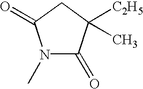 | |
| 12. | NH | H | 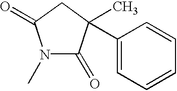 | |
| 13. | NH | H | 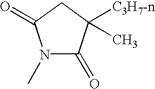 | |
| 14. | NH | H | 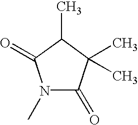 | |
| 15. | NH | H | 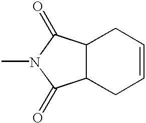 | |
| 16. | NH | H | 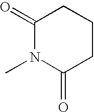 | |
| 17. | NH | H | 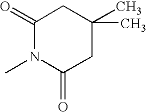 | |
| 18. | NH | H | 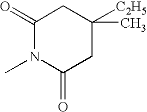 | |
| 19. | NH | H | 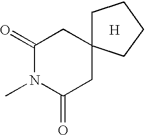 | |
| 20. | NH | H | 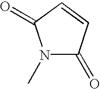 | |
| 21. | NCH₃ | H | 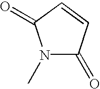 | |
| 22. | NH | H | 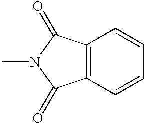 | 151-154 |
| 23. | NCH₃ | H | 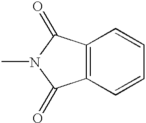 | 1.6108 |
| 24. | NH | H | 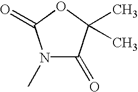 | |
| 25. | NH | H | 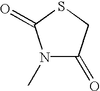 | |
| 26. | NH | H | 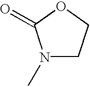 | 132-136 |
| 27. | NCH₃ | H | 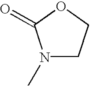 | |
| 28. | NH | H | 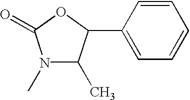 | |
| 29. | NH | H | 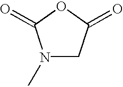 | |
| 30. | NH | H | 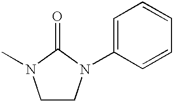 | 166-167 |
| 31. | NCH₃ | H | 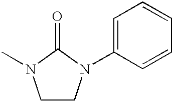 | |
| 32. | NH | H | 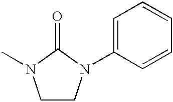 | 175-177 |
| 33. | NCH₃ | H | 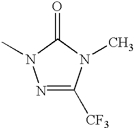 | |
| 34. | NH | H | 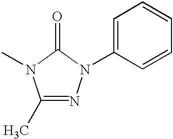 | 152-160 |
| 35. | NCH₃ | H | 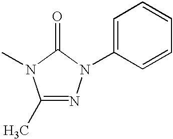 | |
| 36. | NH | H | 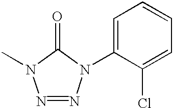 | 62-66 |
| 37. | NCH₃ | H | 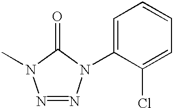 | 1.5989 |
| 38. | NH | H | 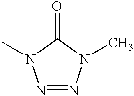 | 149-150 |
| 39. | NCH₃ | H | 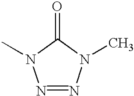 | |
| 40. | NH | H | 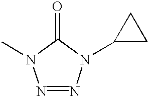 | 173-174 |
| 41. | NCH₃ | H | 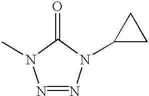 | |
| 42. | NH | H | 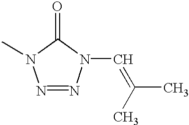 | |
| 43. | NCH₃ | H | 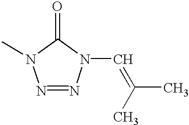 | |
| 44. | NH | H | 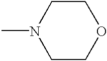 | 1.5742 |
| 45. | NH | H | OH | 137-140 |
| 46. | NH | H | OCH₃ | 79-87 |
| 47. | NH | H | 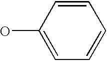 | |
| 48. | NH | CF₃ | OH | 90-93 |
| 49. | NH | CF₃ | OC₂H₅ | 99-101 |
| 50. | NCH₃ | H | OCH₃ | |
| 51. | NCH₃ | H | 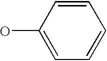 | |
| 52. | NCH₃ | H | 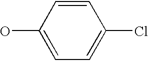 | |
| 53. | NH | H | 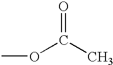 | 86-88 |
| 54. | NH | H | 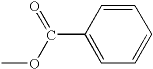 | |
| 55. | NCH₃ | H | OH | |
| 56. | NCH₃ | H | 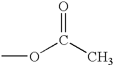 | |
| 57. | NH | H | 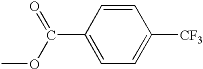 | 120-121 |
| 58. | NH | H | 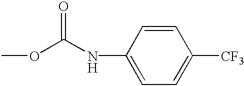 | 117-119 |
| 59. | NH | H | SCH₃ | 74-77 |
| 60. | NH | CF₃ | SCH₃ | |
| 61. | NH | H | 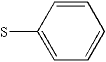 | |
| 62. | NH | CF₃ | 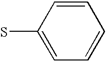 | |
| 63. | NH | H | 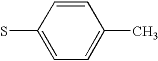 | |
| 64. | NH | CF₃ | 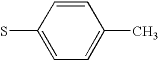 | |
| 65. | NH | H | 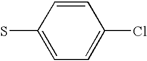 | 106-107 |
| 66. | NH | CF₃ | 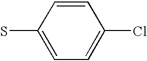 | |
| 67. | NH | H | 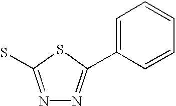 | 170-171 |
| 68. | NH | CF₃ | 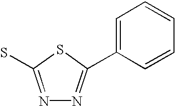 | 124-127 |
| 69. | NH | H | 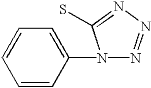 | 137-138 |
| 70. | NH | H | SO₂CH₃ | |
| 71. | NH | H | 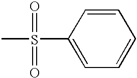 | 175-176 |
| 72. | NH | H | 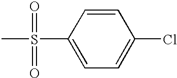 | |
| 73. | NCH₃ | H | SCH₃ | 1.6009 |
| 74. | NCH₃ | H | 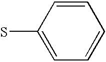 | |
| 75. | NCH₃ | H | 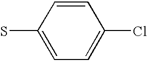 | 1.6310 |
| 76. | NH | H | 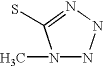 | 176-177 |
| 77. | NH | H | 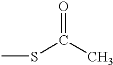 | 1.6049 |
| 78. | NH | H | 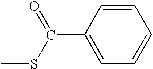 | 84-86 |
| 79. | NCH₃ | H | 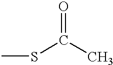 | 1.5935 |
| 80. | NCH₃ | H | 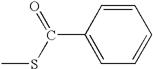 | |
| 81. | NCH₃ | H | PO(OCH₃)₂ | |
| 82. | NCH₃ | H | PO(OC₂H₅)₂ | 1.5292 |
| 83. | NCH₃ | H | 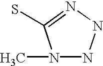 | 100-104 |
| 84. | NCH₃ | H | 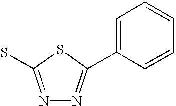 | 1.6712 |
| 85. | NH | H | 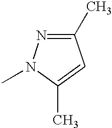 | |
| 86. | NH | H | 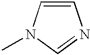 | |
| 87. | NH | H | 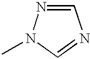 | 142-144 |
| 88. | NH | H | 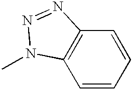 | |
| 89. | NH | C₃H₇-n | 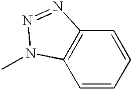 | 1.5759 |
| 90. | NH | C₆H₁₃-n | 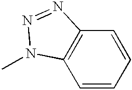 | 1.5720 |
| 91. | NH | C₃H₇-iso | 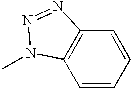 | 152-153 |
| 92. | NH | 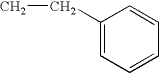 | 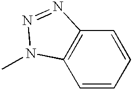 | 1.5720 |
| 93. | NH | 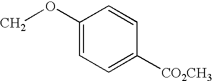 | 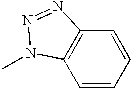 | amorphous |
| 94. | NCH₃ | H | 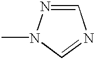 | 98-99 |
| 95. | NH | H | 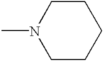 | 75-78 |
| 96. | NH | H | 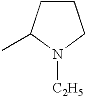 | 1.5553 |
| 97. | O | H | 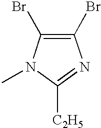 | |
| 98. | O | H | 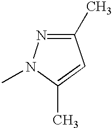 | 80-81 |
| 99. | O | H | 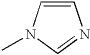 | |
| 100. | O | H | 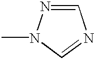 | 106-109 |
| 101. | O | H | 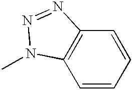 | 112-113 |
| 102. | O | H | 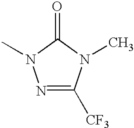 | 90-92 |
| 103. | O | H | 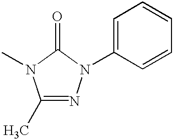 | 102-108 |
| 104. | O | H | 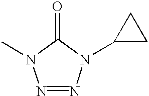 | 1.5640 |
| 105. | O | H | 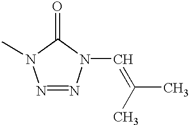 | 75-76 |
| 106. | O | H | 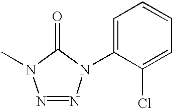 | 104-105 |
| 107. | O | H | 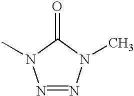 | 81-82 |
| 107a.. | NH | H | 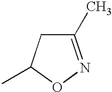 | |
| 107b | 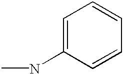 | H | 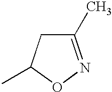 | |
| 107c | NH | CF₃ | 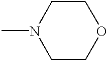 | |
| 107d | NH | CCl₃ | 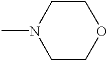 | |
| 107e | NH | CF₃ | OC₃H₇-iso | |
| 107f | NH | CCl₃ | OC₃H₇-iso | |
| 107g | NH | CF₃ | 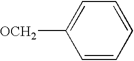 | |
| 107h | NH | CCl₃ | 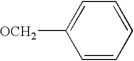 | |
| 107i | NH | CF₃ | OCH₂CF₂CHF₂ | |
| 107j | NH | CCl₃ | OCH₂CF₂CHF₂ | |
| 107k | NH | CF₃ | 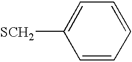 | |
| 107l | NH | H | 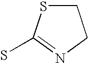 | |

TABLE 2

| | |
|---|---|
| | (Ie) |
| 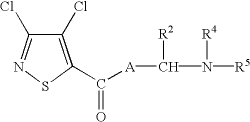 | |

| Com- | | | | | Melting |
|---|---|---|---|---|---|
| pound | | | | | point ° C. or |
| No. | A | R² | R⁴ | R⁵ | n₂₀^{D} |
| 108. | NH | H | 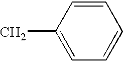 | CHO | 102-103 |
| 109. | NH | H | 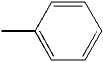 | CHO | 1.6206 |
| 110. | NH | H | 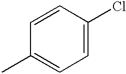 | CHO | 137-138 |
| 111. | NH | H | 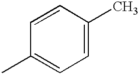 | CHO | |
| 112. | NH | C₃H₇-n | H | COCH₃ | 162-165 |
| 113. | NH | H | CH₃ | COCH₃ | 1.5740 |
| 114. | NH | H | C₂H₅ | COCH₃ | |
| 115. | NH | H | C₃H₇-n | COCH₃ | |
| 116. | NH | H | C₃H₇-iso | COCH₃ | |
| 117. | NH | H | 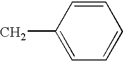 | COCH₃ | |
| 118. | NH | H | 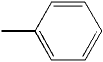 | COCH₃ | |
| 119. | NH | H | 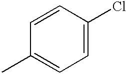 | COCH₃ | |
| 120. | NH | H | 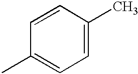 | COCH₃ | |
| 121. | NH | H | CH₃ | COC₄H₉-tert | 100-101 |
| 122. | NH | H | CH₃ | 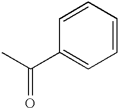 | 1.5921 |
| 123. | NH | H | C₂H₅ | 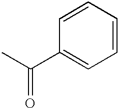 | |
| 124. | NH | H | C₃H₇-n | 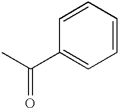 | |
| 125. | NH | H | C₃H₇-iso | 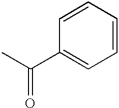 | |
| 126. | NH | H | 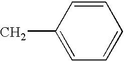 | 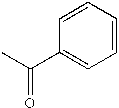 | 98-102 |
| 127. | NH | H | 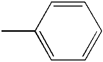 | 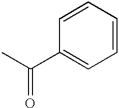 | |
| 128. | NH | H | CH₃ | 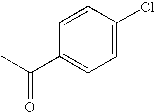 | 1.6040 |
| 129. | NH | H | CH₃ | 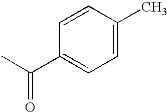 | |
| 130. | NCH₃ | H | CH₃ | CHO | |
| 131. | NCH₃ | H | C₂H₅ | CHO | |
| 132. | NCH₃ | H | C₃H₇-n | CHO | |
| 133. | NCH₃ | H | C₃H₇-iso | CHO | |
| 134. | NCH₃ | H | 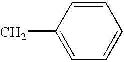 | CHO | |
| 135. | NCH₃ | H | 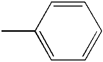 | CHO | |
| 136. | NCH₃ | H | 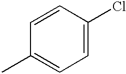 | CHO | |
| 137. | NCH₃ | H | CH₃ | COCH₃ | 1.5552 |
| 138. | NCH₃ | H | C₂H₅ | COCH₃ | |
| 139. | NCH₃ | H | C₃H₇-n | COCH₃ | |
| 140. | NCH₃ | H | C₃H₇-iso | COCH₃ | |
| 141. | NCH₃ | H | 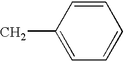 | COCH₃ | |
| 142. | NCH₃ | H | 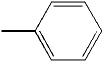 | COCH₃ | |
| 143. | NCH₃ | H | 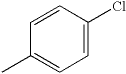 | COCH₃ | |
| 144. | NCH₃ | H | 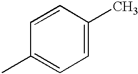 | COCH₃ | |
| 145. | NCH₃ | H | CH₃ | 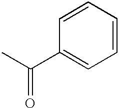 | |
| 146. | NCH₃ | H | C₂H₅ | 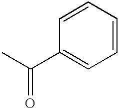 | |
| 147. | NCH₃ | H | C₃H₇-n | 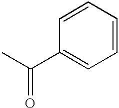 | |
| 148. | NCH₃ | H | C₃H₇-iso | 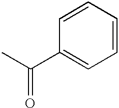 | |
| 149. | NCH₃ | H | 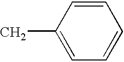 | 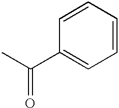 | |
| 150. | NCH₃ | H | 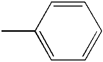 | 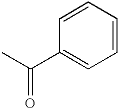 | 45-48 |
| 151. | NCH₃ | H | CH₃ | 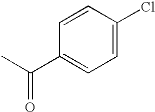 | 36-38 |
| 152. | NCH₃ | H | CH₃ | 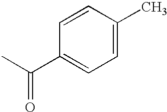 | |
| 153. | NH | H | H | 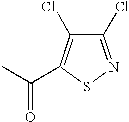 | 175-177 |
| 154. | NH | H | 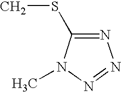 | 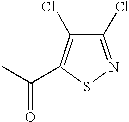 | 185-188 |
| 155. | S | H | CH₃ | COCH₃ | 1.6012 |
| 156. | NH | H | CH₃ | SO₂CH₃ | 113-114 |
| 157. | NH | H | CH₃ | 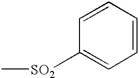 | 111-115 |
| 158. | NCH₃ | H | CH₃ | SO₂CH₃ | |
| 159. | NCH₃ | H | CH₃ | 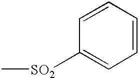 | 76-78 |

TABLE 3

| | |
|---|---|
| | (If) |
| 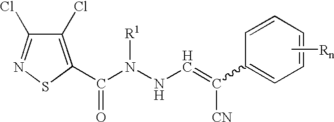 | |

| | | | Melting point (mp)° C. |
|---|---|---|---|
| Compound | | | or |
| No. | R¹ | Rₙ | n₂₀^{D} |
| 160. | H | H | 212-214 |
| 161. | CH₃ | H | 130-139 |
| 162. | H | 2-F | 230-232 |
| 163. | H | 3-F | 218-221 |
| 164. | H | 4-F | 220-222 |
| 165. | H | 2-Cl | 216-218 |
| 166. | H | 3-Cl | 220 |
| 167. | H | 4-Cl | 216 |
| 168. | H | 2-Br | |
| 169. | H | 3-Br | |
| 170. | H | 4-Br | 230-231 |
| 171. | H | 2-I | |
| 172. | H | 2-CN | |
| 173. | H | 2-CH₃ | |
| 174. | H | 3-CH₃ | |
| 175. | H | 4-CH₃ | 192-196 |
| 176. | H | 4-C₂H₅ | |
| 177. | H | 4-C₃H₇-iso | |
| 178. | H | 2-CF₃ | |
| 179. | H | 3-CF₃ | |
| 180. | H | 4-CF₃ | |
| 181. | H | 2-OCH₃ | |
| 182. | H | 3-OCH₃ | |
| 183. | H | 4-OCH₃ | 218-219 |
| 184. | H | 3-OCF₃ | |
| 185. | H | 4-OCF₃ | |
| 186. | H | 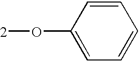 | |
| 187. | H | 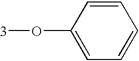 | |
| 188. | H | 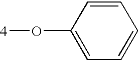 | |
| 189. | H | 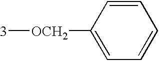 | |
| 190. | H | 2,3,-F₂ | |
| 191. | H | 2,4,-F₂ | >250 |
| 192. | H | 2,5-F₂ | |
| 193. | H | 3,4-F₂ | |
| 194. | H | 3,5-F₂ | |
| 195. | H | 2,4-Cl₂ | 217-219 |
| 196. | H | 3,4-Cl₂ | 237-239 |
| 197. | H | 2,5-(CH₃)₂ | |
| 198. | H | 3,5-(CH₃)₂ | |
| 199. | H | 3,5-(CF₃)₂ | |
| 200. | H | 3,4-(OCH₃)₂ | 212-214 |
| 201. | H | 3,5-(OCH₃)₂ | |
| 202. | H | 3,4-(CH₂OCH₂) | |
| 203. | H | 3,4,5-(OCH₃)₃ | |
| 204. | H | 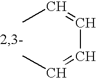 | |
| 205. | H | 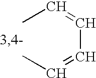 | |

TABLE

| | |
|---|---|
| | (I) |
| 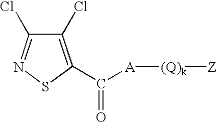 | |

| Com- | | | Melting point (mp) ° C. |
|---|---|---|---|
| pound | | | or |
| No. | A | -(Q)ₖZ | n₂₀^{D} |
| 206. | NH | CH₂CH═CH₂ | 65-66 |
| 207. | NH | CH(CH₃)CH═CH₂ | |
| 208. | NH | CH₂C(CH₃)═CH₂ | |
| 209. | NCH₃ | CH₂CH═CH₂ | |
| 210. | NC₂H₅ | CH₂CH═CH₂ | |
| 211. | 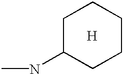 | CH₂CH═CH₂ | |
| 212. | 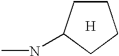 | CH₂CH═CH₂ | |
| 213. | NH | 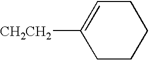 | 1.5723 |
| 214. | 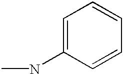 | CH₂CH═CH₂ | 1.6012 |
| 215. | NC₃H₇-iso | CH₂C(CH₃)═CH₂ | 1.5480 |
| 216. | NH | 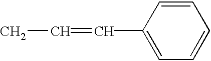 | |
| 217. | NH | CH₂CH═CHCH₃ | |
| 218. | NH | CH₂CH(OH)CH₂OH | 68-72 |
| 219. | 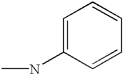 | CH₂CH(OH)CH₂OH | 1.5962 |
| 220. | NC₃H₇-iso | 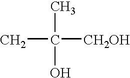 | 126-127 |
| 221. | NH | 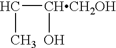 | |
| 222. | NH | 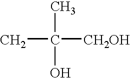 | |
| 223. | NH | 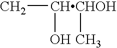 | |
| 224. | NCH₃ | CH₂CH(OH)CH₂OH | |
| 225. | 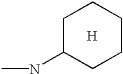 | CH₂CH(OH)CH₂OH | |
| 226. | 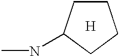 | CH₂CH(OH)CH₂OH | |
| 227. | NC₂H₅ | 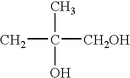 | |
| 228. | NH | 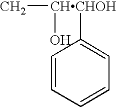 | |
| 229. | NH | 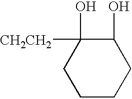 | 107-108 |
| 230. | NH | 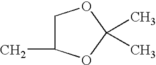 | 132-133 |
| 231. | NH | 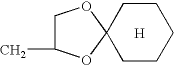 | 1.5526 |
| 232. | NH | 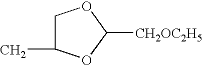 | 1.5570 |
| 233. | NH | 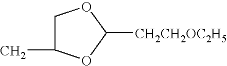 | 1.5268 |
| 234. | NH | 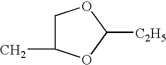 | 1.5452 |
| 235. | NH | 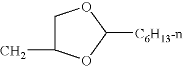 | 1.5247 |
| 236. | NH | 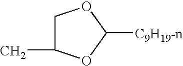 | 1.5155 |
| 237. | NH | 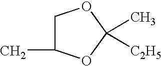 | 1.5440 |
| 238. | NH | 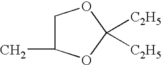 | 1.5375 |
| 239. | NH | 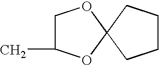 | 1.5560 |
| 240. | NH | 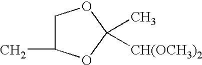 | 1.5340 |
| 241. | NH | 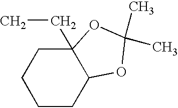 | 1.5382 |
| 242. | NH | 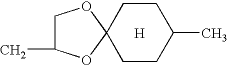 | 1.5526 |
| 243. | NH | 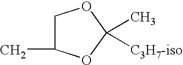 | 1.5381 |
| 244. | NH | 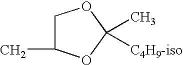 | 1.5332 |
| 245. | O | 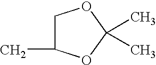 | 1.5285 |
| 245a. | NC₂H₅ | CH₂C(CH₃)═CH₂ | |
| 245b. | O | 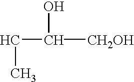 | |
| 245c. | O | 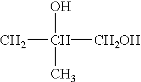 | |
| 245d. | NH | 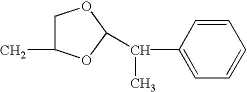 | |
| 245e. | NH | 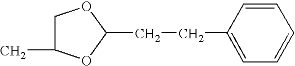 | |
| 245f. | NH | 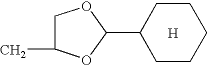 | |
| 245g. | NH | 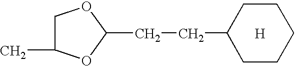 | |

TABLE 5

| | |
|---|---|
| | (I) |
| 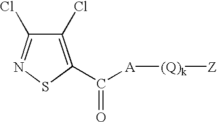 | |

| | | | Melting |
|---|---|---|---|
| Com- | | | point |
| pound | | | (mp)(° C.) or |
| No. | A | (-Q)ₖ-Z | n₂₀^{D} |
| 246. | NH | C(CH₃)₂CH₂OH | 1.5722 |
| 247. | NH | (CH₂)₃OH | 70-71 |
| 248. | NH | CH₂CHCH₃OH | 82-86 |
| 249. | NH | CH₂CH(C₂H₅)OH | 91-33 |
| 250. | NH | 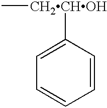 | 83-86 |
| 251. | NC₂H₅ | CH₂CH₂OH | 1.5680 |
| 252. | NC₃H₇-iso | CH₂CH₂OH | 88-91 |
| 253. | NH | 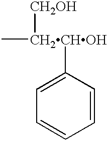 | 1.5843 |
| 254. | NH | 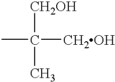 | 93-95 |
| 255. | NH | CH(CH₃)CH₂OH | 108-109 |
| 256. | NH | CH(C₃H₇-iso)CH₂OH | 1.5586 |
| 257. | NCH₃ | CH₂CH(C₄H₉-tert)OH | 83-87 |
| 258. | NH | 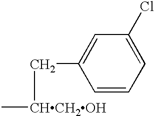 | 122-129 |
| 259. | NCH₂CH₂OH | CH₂CH₂OH | 1.5550 |
| 260. | O | 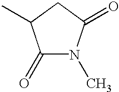 | 156-158 |
| 261. | O | 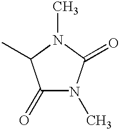 | 115-117 |
| 262. | NH | CH₂CH₂NHCH₃ | 81-83 |
| 263. | NH | CH₂CH₂N(CH₃)₂ | |
| 264. | NH | (CH₂)₃NHCH₃ | 1.5699 |
| 265. | NH | 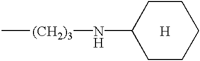 | 63-65 |
| 266. | NH | (CH₂)₃N(CH₃)₂ | |
| 267. | NH | CH(CH₃)(CH₂)₃N(C₂H₅)₂ | |
| 268. | NH | (CH₂)₂NHCH₂CH₂OH | 1.5583 |
| 269. | NH | (CH₂)₂NHCH₂CH(CH₃)OH | 96-99 |
| 270. | NH | 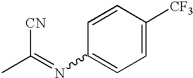 | 151-153 |
| 271. | NH | 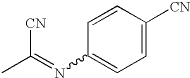 | 168-172 |
| 272. | NH | 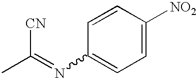 | 169-175 (decomp.) |
| 273. | S | H | 79-80 |
| 273a. | NH | CH₂CH₂N(C₂H₅)₂ | |

TABLE 6
(Ig)

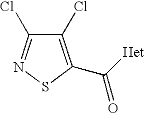

| Com- | | |
|---|---|---|
| pound | | Melting point |
| No. | Het | (mp(° C.) or n₂₀^{D} |
| 274. | 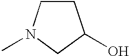 | 1.5622 |
| 275. | 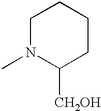 | 1.5831 |
| 276. | 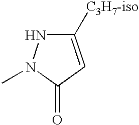 | 181-185 |
| 277. | 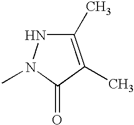 | >250 |
| 278 | 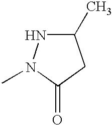 | 211-214 |
| 279. | 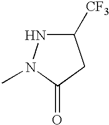 | 198-199 |
| 280. | 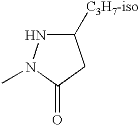 | 163-164 |
| 281. | 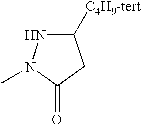 | 160-163 |
| 282. | 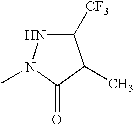 | 191-193 |
| 283. | 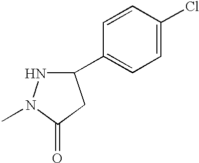 | 164-167 |
| 284. | 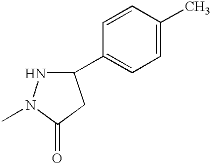 | |
| 285. | 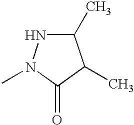 | 193-194 |
| 286. | 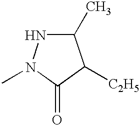 | 190-194 |
| 287. | 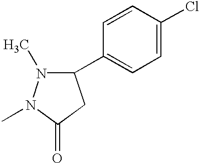 | 140-141 |
| 288. | 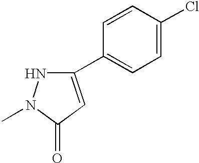 | >250 |
| 289. | 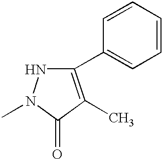 | 190-191 |
| 289a. | 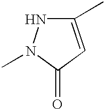 | |

TABLE 7
(Ih)

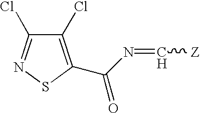

| Compound | | Melting point (mp)(° C.) |
|---|---|---|
| No. | Z | or n₂₀^{D} |
| 290. | N(CH₃)₂ | 196-197 |
| 291. | N(C₂H₅)₂ | |
| 292. | N(C₃H₇-iso)₂ | |
| 293. | N(C₄H₉-n)₂ | |
| 294. | N(C₃H₇-iso)CH₃ | |
| 295. | 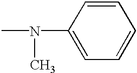 | |
| 296. | 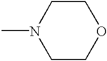 | |
| 297. | 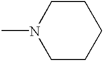 | |

Preparation of Intermediates

### Synthesis Example 27

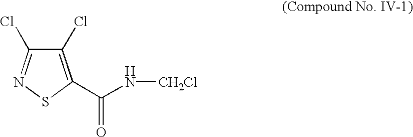

A suspension, obtained by adding 3,4-dichloro-5-isothiazolecarboxamide (2.0 g) and paraformaldebyde (0.3 g) to chlorotrimethylsilane (20 ml) was refluxed in a sealed tube for 3 hours by heating. After the solvent was distilled off, methylene chloride was added and the insoluble product was filtered off. The solvent was distilled off under reduced pressure to obtain N-chloromethyl-3,4-dichloro-5-isothiazolecarboxamide (2.0 g).

mp 98-99.

The intermediate compounds obtained in a similar manner as the above-mentioned Synthesis Example 27 are shown, together with the compound synthesized in Synthesis Example 27, in the following Table 8.

TABLE 8
(IV)

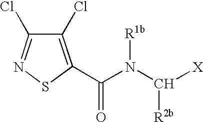

| | | | | Melting point |
|---|---|---|---|---|
| Compound | | | | (mp(° C.) |
| No. | R^{1b} | R^{2b} | X | n₂₀^{D} |
| IV-1 | H | H | Cl | 98-99 |
| IV-2 | CH₃ | H | Cl | 1.5818 |
| IV-3 | H | CF₃ | Cl | |

### BIOLOGICAL TEST EXAMPLES

### Test Example A

Test of foliar spray effect against Pyricularia oryzae

Preparation of Formulations of the Compounds Tested

Active compound: 30-40 parts by weight

Carrier: mixture of diatomaceous earth and kaolin (1:5), 55-65 parts by weight

Emulsifier: polyoxyethylene alkyl phenyl ether, 5 parts by weight

The above-mentioned amounts of active compound, carrier and emulsifier are crushed and mixed to make a wettable powder. A portion of the wettable powder comprising the prescribed amount of active compound is diluted with water and used for testing.

Testing Procedure

Seedlings of paddy rice (cultivar: Kusabue) were cultivated in plastic pots each having a diameter of 6 cm. The previously prepared solution of the prescribed concentration of active compound was sprayed over the seedlings in the 1.5-2 leaf stage, at a rate of 20 ml per 3 pots. 5 days after the application, a suspension of spores of artificially cultured Pyricularia oryzae was sprayed on the test plants once for inoculation, and the plants were kept at 25° C. and 100% relative humidity for infection. 7 days after the inoculation, the infection rate per pot was classified and evaluated according to the following standard and the control value (%) was calculated. Phytotoxicity was tested at the same time. This test is an average of the results of 3 replications. The evaluation of the infection rate and the calculation method of the control value are identical in each of the Test Examples A-D.

| Infection rate | Percentage of lesion area in (%) |
|---|---|
| 0 | 0 |
| 0.5 | less than 2 |
| 1 | 2-less than 5 |
| 2 | 5-less than 10 |
| 3 | 10-less than 20 |
| 4 | 20-less than 40 |
| 5 | more than 40 |

$$Control value ( \% ) = ( 1 - \frac{Infection rate of treated section}{Infection rate of untreated section} ) \times 100$$

Test Results

Compounds No. 7, 26, 32, 34, 37, 38, 45, 48, 52, 59, 65, 67, 71, 73, 75, 76, 89, 93, 98, 100, 101, 103, 104, 105, 106, 107, 108, 112, 113, 121, 128, 151 and 153 showed control values of more than 80% at an active compound concentration of 500 ppm. No phytotoxicity was observed.

### Test Example B

Test of water surface application effect against Pyricularia oryzae.

Testing Procedure

Seedlings of paddy rice (cultivar: Kusabue) in the 1.5 leaf stage were cultivated in plastic pots each having a diameter of 6 cm. The seedlings were then transplanted into irrigated plastic cups each having a diameter of 10 cm, one seedling per pot, and the water just covering the soil. The solution of the prescribed concentration of the active compound, which had been prepared in the same manner as that of Test Example A, was dropped to the water surface with a pipette at a rate of 5 ml per pot. 7 days after the chemical treatment, a suspension of spores of artificially cultured Pryricularia oryzae was sprayed once on the test plants for inoculation, and the plants were kept at a temperature of 25° C. and a relative atmospheric humidity of 100%. Seven days after the inoculation, the infection rate per pot was classified and evaluated, and further the control value (%) was calculated. Phytotoxicity was tested at the same time.

This test is an average of the results of 3 replications.

Test Results

Compounds No. 1, 7, 22, 26, 30, 38, 48, 49, 67, 73, 75, 76, 92, 98, 100, 101, 103, 104, 105, 106, 107, 108, 112, 113, 121, 122, 128, 137 and 154 showed control values of more than 80% at an active compound rate of 8 kg/ha. No phytotoxicity was observed.

### Test Example C

Test for the effect of seed treatment against Pyricularia oryzae

Testing Procedure

Seeds of paddy rice (cultivar: Kasabue) were soaked in a diluted solution of an active compound having the prescribed concentration. 5 ml of such solution, which had been prepared in the same manner as that of Test Example A, were used per 150 grains of seed. Soaking was conducted at a temperature of 20° C. for 5 days. After the soaking, the air-dried seeds were sown in 2 plastic pots, each having a diameter of 9 cm, and the seeds were germinated by placing the pots in a warmed nursery box (32° C.) for 3 days. After cultivating the seedlings for 2 weeks, the plants reached the 2-2.5 leaf stage. A spore suspension of artificially cultured Pyricularia oryzae was then sprayed on the test plants once, and the plants were kept at a temperature of 25° C. and a relative atmospheric humidity of 100% for infection. Seven days after the inoculation, the infection rate per pot was classified and evaluated and the control value (%) was calculated. Phytotoxicity was tested at the same time.

This test is an average of the results of 2 replications.

Test Results

Compounds No. 1, 7, 26, 30, 38, 49, 58, 76, 89, 92, 93, 106, 108, 109, 110, 112, 113, 121, 126, 128, 137, 150, 153, 154, 288 and 290 showed control values of more than 80% at an active compound concentration of 500 ppm. No phytotoxicity was observed.

### Test Example D

Spraying test against Phytophthora infestans.

Testing Procedure

About 1 seed of tomato (cultivar: Regina) was sown in each plastic pot of a diameter of 6 cm, and raised in a greenhouse at 15-25° C. The solution obtained by diluting the prepared formulation of the test compound to the prescribed concentration as mentioned above, was sprayed at a rate of 20 ml per 3 pots over seedlings which had reached the 4 leaf stage. Zoosporangia formed on the lesion of tomato plants, which previously had been infected with Phytophthora infestans, were washed down with a brush into distilled water to make a suspension. Five days after the tomato plants had been sprayed with the solution of active compound, the suspension was sprayed on the plants once for inoculation, and the treated plants were kept at a temperature of 20° C. and a relative atmospheric humidity of 100%. Four days after the inoculation, the infection rate per pot was classified and the control value (%/0) was calculated. Phytotoxicity was tested at the same time.

This test is an average of the results of 3 replications.

Test Results

Compounds No. 22, 30, 34, 37, 38, 45, 52, 58, 65, 69, 71, 89, 90, 92, 93, 100, 103, 104, 106, 107, 108, 113, 137, 150, 151, 153 and 290 showed control values of more than 80% at an active compound concentration of 500 ppm. No phytotoxicity was observed.

### FORMULATION EXAMPLES

### Formulation Example I

### Granules

25 parts by weight of water were added to a mixture of 10 parts by weight of Compound No. 30 according to the invention, 30 parts by weight of bentonite (montmorillonite), 58 parts by weight of talc and 2 parts by weight of lignin sulphonic acid salt, and the mixture was kneaded thoroughly. The resulting product was granulated by means of an extrusion granulator to form granules having a size of from 10 to 40 meshes. The granules were dried at a temperature between 40 and 50° C.

### Formulation Example II

### Granules

95 parts by weight of a clay mineral having a particle size distribution within a range of from 0.2 to 2 mm were introduced into a rotary mixer. This product was uniformly wetted by spraying thereto under rotation a mixture of 5 parts by weight of Compound No. 38 according to the invention and a liquid diluent. The granules obtained in this manner were dried at a temperature between 40 and 50° C.

### Formulation Example III

### Emulsifiable Concentrate

An emulsifiable concentrate was prepared by mixing 30 parts by weight of Compound No. 106 according to the invention, 5 parts by weight of xylene, 8 parts by weight of polyoxyethylene alkyl phenyl ether and 7 parts by weight of calcium alkylbenzene sulphonate with stirring.

### Formulation Example IV

### Wettable Powder

A wettable powder was prepared by thoroughly mixing 15 parts by weight of Compound No. 108 according to the invention, 80 parts by weight of a mixture (1:5) of White Carbon (fine powder of hydrated non-crystalline silicon oxide) and powdery clay, 2 parts by weight of sodium alkylbenzene sulphonate and 3 parts by weight of a condensate of sodium alkylnaphthalene sulphonate and formaldehyde in powdery state.

### Formulation Example V

### Wettable Granules

20 parts by weight of Compound No. 113 according to the invention, 30 parts by weight of sodium lignin sulphonate, 15 parts by weight of bentonite and 35 parts by weight of calcined diatomaceous earth powder were thoroughly mixed with water. The resulting product was granulated by means of extrusion through a 0.3 mm screen. After drying the product, wettable granules were obtained.

1. Isothiazolecarboxylic acid derivatives of the formula 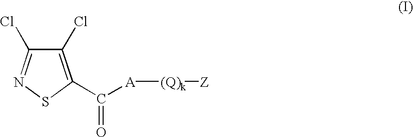
wherein
A represents an oxygen atom, a sulphur atom or a group of the formula 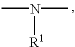
in which
R¹ represents a hydrogen atom, C₁₋₄ alkyl, C₃₋₆ cycloalkyl, phenyl or 2-hydroxyethyl,
Q represents a group selected from 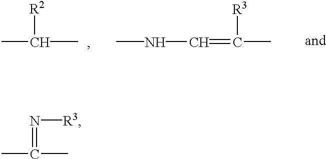
in which
R² represents a hydrogen atom, C₁₋₄ alkyl, C₁₋₄ haloalkyl, C₇₋₉ aralkyl or phenoxymethyl, which may be substituted by C₁₋₄ alkoxy-carbonyl, and
R³ represents phenyl, optionally substituted by halogen, C₁₋₄ alkyl, C₁₋₄ haloalkyl, C₁₋₄ alkoxy, C₁₋₄ haloalkyl, phenoxy, benzyloxy, cyano, oxydimethylene and/or nitro, or represents naphthyl,
k represents 0 or 1, and
Z represents a 5-7-membered heterocyclic ring comprising 1 to 4 nitrogen atoms, wherein the heterocyclic ring may be substituted by one or more substituents selected from halogen, C₁₋₄ alkyl, C₁₋₄ alkoxy, C₁₋₄ haloalkoxy, C₃₋₆ cycloalkoxy, C₂₋₄ alkenyl, phenyl, halophenyl, oxo and/or spiro-bonded C₃₋₆ alicyclic groups, and wherein the heterocyclic ring may be condensed with a benzene or cyclohexene ring, or
Z represents a 5-7-membered heterocyclic ring comprising at least one nitrogen atom and one oxygen atom, or comprising at least one nitrogen atom and one sulphur atom, wherein each of the heterocyclic rings may be substituted by one or more substituents selected from C₁₋₄ alkyl, phenyl and/or oxo, or
Z represents cyano or a group selected from 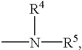
—O—R⁶, —S(O)ₘ—R⁷ or 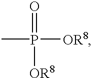
in which
R⁴ represents a hydrogen atom, C₁₋₄ alkyl, benzyl or phenyl, the last two radicals being optionally substituted by one to three radicals selected from halogen and/or C₁₋₄ alkyl, or
R⁴ represents tetrazol-5-yl-thiomethyl, which may be substituted by C₁₋₄ alkyl,
R⁵ represents formyl, C₁₋₄ alkylcarbonyl, 3-4-dichloroisothiazol-5-yl-carbonyl, C₁₋₄ alkylsulphonyl or phenylsulphonyl or
R⁵ represents phenylcarbonyl, optionally substituted by one to three radicals selected from halogen and C₁₋₄ alkyl,
R⁶ represents a hydrogen atom, C₁₋₄ alkyl, C₁₋₄ haloalkyl, benzyl, halogen-substituted benzyl, phenyl, halogen-substituted phenyl, C₁₋₄ alkylcarbonyl, benzoyl, C₁₋₄ haloalkyl-substituted benzoyl, phenylcarbamoyl or C₁₋₄ haloalkyl-substituted phenylcarbamoyl,
R⁷ represents C₁₋₄ alkyl, benzyl or phenyl the last two radicals being optionally substituted by one to three radicals selected from C₁₋₄ alkyl and/or halogen, or
R⁷ represents tetrazol-5-yl or
R⁷ represents thiadiazol-2-yl optionally substituted by C₁₋₄ alkyl or phenyl, or
R⁷ represents 2-thiazoline-2-yl, C₁₋₄ alkylcarbonyl or benzoyl,
m represents 0, 1 or 2, and
R⁸ represents C₁₋₄ alkyl,
or, in case
A represents a 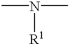
group,
then
R¹, Q and Z may represent a 5- or 6-membered heterocyclic group comprising 1-3 nitrogen atoms and being optionally substituted by one to three radicals selected from C₁₋₄ alkyl, C₁₋₄ haloalkyl, hydroxy, oxo, hydroxymethyl or phenyl, which in turn may be substituted by halogen and/or C₁₋₄ alkyl, or
-(Q)ₖ-Z represents a group selected from 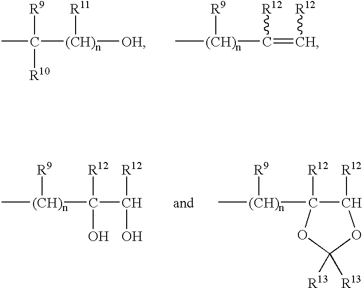
wherein
n represents 1 or 2,
R⁹ represents a hydrogen atom or C₁₋₄ alkyl,
R¹⁰ represents a hydrogen atom, hydroxymethyl or benzyl which may be substituted by 1 to 3 halogen atoms,
R¹¹ represents a hydrogen atom, C₁₋₄ alkyl or phenyl,
R¹² represents a hydrogen atom, C₁₋₄ alkyl or phenyl, or two of the R¹² radicals, together with the carbon atoms to which they are bonded, may form a 5- or 6-membered hydrocarbon ring, and
R¹³ represents a hydrogen atom, C₁₋₉ alkyl, C₃₋₆ cycloalkyl, C₇₋₈ arylalkyl, C₃₋₆ cycloalkyl-C₁₋₄ alkyl, C₁₋₄ alkoxy-C₁₋₄ alkyl or di-(C₁₋₄ alkoxy)-methyl, or the two R¹³ radicals, together with the carbon atom to which they are bonded, form a C₅₋₆ alicyclic ring which is optionally substituted by C₁₋₄ alkyl, or
-A-(Q)ₖ-Z represents —SH or a group of the formula 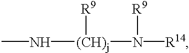
in which
R⁹ has the above-mentioned meanings,
R¹⁴ represents C₁₋₄ alkyl, C₃₋₆ cycloalkyl or hydroxy-substituted C₂₋₄ alkyl, and
j represents 2, 3 or 4,
or, in case
A represents 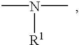
Q represents 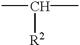
and
Z represents 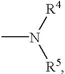
these
radicals together may represent a group of the formula 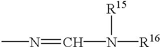
in which
R¹⁵ and R¹⁶ independently of one another represent C₁₋₄ alkyl or phenyl or
R¹⁵ and R¹⁶ together with the nitrogen atom, to which they are bonded, form a 5- or 6-membered heterocyclic group comprising at least one nitrogen atom or comprising at least one nitrogen atom and one oxygen atom,
with the proviso that
in case
Q represents a group of the formula 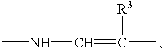
then
A represents 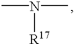
wherein
R¹⁷ represents a hydrogen atom or C₁₋₄ alkyl, and
Z represents cyano,
and in case
Q represents a group of the formula 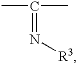
then
A represents —NH and
Z represents cyano
and in case
-(Q)ₖ-Z represents 2,3-dihydroxypropyl, then
A represents a sulphur atom or a group of the formula 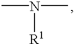
and in case
-(Q)ₖ-Z represents 2-hydroxyethyl and
A represents a group of the formula, then
R¹ represents C₁₋₄ alky, C₃₋₆ cycloalkyl, phenyl or 2-hydroxyethyl,
and in case
A represents a group of the formula 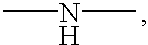
then
Q represents —CH₂— and
Z represents a group of the formula 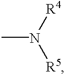
in which
R⁴ represents a hydrogen atom, benzyl or phenyl, the last two radicals being optionally substituted by halogen and/or C₁₋₄ alkyl,
and with the further proviso that
Z does not represent cyano or a group selected from 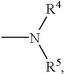
—OR⁶, —S(O)ₘ—R and 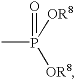
if
A is oxygen or sulphur and
k is o.

2. Isothiazolecarboxylic acid derivatives of the formula (I) according to claim 1, in which
A is an oxygen atom, a sulphur atom or a group of the formula 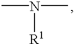
in which
R¹ represents a hydrogen atom, C₁₋₃ alkyl, cyclopentyl, cyclohexyl, phenyl or 2-hydroxyethyl,
Q represents a group selected from 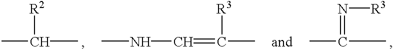
in which
R² represents a hydrogen atom, C₁₋₆ alkyl, haloalkyl with 1 to 3 carbon atoms and 1 to 5 fluorine, chlorine and/or bromine atoms, C₇₋₈ aralkyl or phenoxymethyl, which may be mono-or di-substituted by C₁₋₃ alkoxy-carbonyl, and
R³ represents phenyl, which may be substituted by 1 to 3 radicals selected from, fluoro, chloro, bromo, C₁₋₃ alkyl, haloalkyl with 1 to 3 carbon atoms and 1 to 5 fluorine, chlorine and/or bromine atoms, C₁₋₃ alkoxy, haloalkoxy with 1 to 3 carbon atoms and 1 to 5 fluorine, chlorine and/or bromine atoms, phenoxy, benzyloxy, cyano and/or nitro, or may be mono-substituted by oxydimethylene, or represents naphthyl,
k represents o or 1, and
Z represents a 5-7-membered heterocyclic ring comprising 1 to 4 nitrogen atoms, wherein the heterocyclic ring may be substituted by up to 3 substituents selected from fluorine, chlorine, bromine, C₁₋₃ alkyl, methoxy, ethoxy, haloalkyl with 1 to 3 carbon atoms and 1 to 5 fluorine, chlorine and/or bromine atoms, cyclopropyl, cyclopentyl, C₃₋₄ alkenyl, phenyl and/or halophenyl comprising 1 to 3 fluorine and/or chlorine atoms, and wherein the heterocyclic ring may also be mono- or disubstituted by oxo or spiro-bonded C₃₋₅ alicyclic groups and wherein the heterocyclic ring may be condensed with a benzene or cyclohexene ring,
Z represents a 5- or 6-membered heterocyclic ring comprising at least one nitrogen atom and one oxygen atom, or comprising at least one nitrogen atom and one sulphur atom, wherein each of the heterocyclic rings may be substituted by 1 to 3 substitutents selected from C₁₋₃ alkyl and/or phenyl, and may also be substituted by 1 or 2 oxo groups, or
Z represents cyano or a group selected from 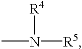
—O—R⁶, —S(O)ₘ—R⁷ and 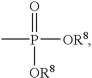
in which
R⁴ represents a hydrogen atom, C₁₋₃ alkyl, benzyl or phenyl, the last two radicals being optionally substituted by 1 to 3 radicals selected from fluorine, chlorine, methyl and/or ethyl, or
R⁴ represents tetrazol-5-yl-thiomethyl, which may be substituted by C₁₋₃ alkyl,
R⁵ represents formyl, C₁₋₄ alkyl, carbonyl, 3,4-dichloroisothiazol-5-ylcarbonyl, C₁₋₂ alkylsuphonyl or phenylsulphonyl, or
R⁵ represents phenylcarbonyl, optionally substituted by 1 to 3 radicals selected from fluorine, chlorine and/or C₁₋₄ alkyl,
R⁶ represents a hydrogen atom, C₁₋₃ alkyl, C₁₋₃ fluoroalkyl, or represents benzyl or phenyl, each of which may be substituted by 1 to 3 radicals selected from fluorine and/or chlorine, or represents acetyl or propionyl, or,
represents benzoyl or phenylcarbamoyl, each of which may be substituted by 1 to 3 radicals selected from haloalkyl with 1 to 3 carbon atoms and 1 to 3 fluorine, chlorine and/or bromine atoms,
R⁷ represents C₁₋₃ alkyl, benzyl or phenyl the last two radicals being optionally substituted by one to three radicals selected from C₁₋₃ alkyl, fluorine and/or chlorine, or
R⁷ represents tetrazol-5-yl or
R⁷ represents thiadiazol-2-yl optionally substituted by C₁₋₃ alkyl or phenyl, or
R⁷ represents 2-thiazoline-2-yl, C₁₋₂ alkylcarbonyl or benzoyl,
m represents 0 or 2, and
R⁸ represents methyl or ethyl,
or, in case
A represents a 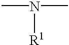
group,
then
R¹, Q and Z together with the nitrogen atom of the 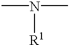
group may represent a 5- or 6-membered heterocyclic group comprising 1 to 3 nitrogen atoms and being optionally substituted by 1 to 3 radicals selected from C₁₋₄ alkyl, haloalkyl, with 1 to 3 carbon atoms and 1 to 5 fluorine, chlorine and/or bromine atoms, hydroxy, oxo, hydroxymethyl and/or phenyl, which in turn may be substituted by 1 to 3 radicals selected from fluorine, chlorine and/or C₁₋₄ alkyl, or
-(Q)ₖ-Z represents a group selected from 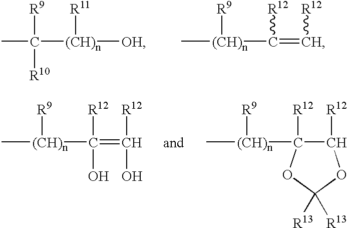
wherein
n represents 1 or 2,
R⁹ represents a hydrogen atom, C₁₋₃ alkyl,
R¹⁰ represents a hydrogen atom, hydroxymethyl or benzyl, which may be substituted by 1 to 3 chlorine atoms,
R¹¹ represents a hydrogen atom, methyl, ethyl, n-propyl, iso-propyl, tert-butyl or phenyl,
R¹² represents a hydrogen atom, C₁₋₃ alkyl or phenyl, or two of the R¹² radicals, together with the carbon atoms to which they are bonded, may form a 5- or 6-membered hydrocarbon ring, and
R¹³ represents a hydrogen atom, C₁₋₆ alkyl, cyclohexyl, 2-phenethyl, α-methylbenzyl, 2-cyclohexylethyl, C₁₋₃ alkoxy-C₁₋₃ alkyl or di(C₁₋₂ alkoxy)methyl, or the two R¹³ radicals, together with the carbon atom to which they are bonded, form a C₅₋₆ alicyclic ring which is optionally substituted by C₁₋₃ alkyl, or
-A-(Q)ₖ-Z represents —SH or a group of the formula 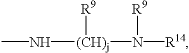
in which
R⁹ has the above-mentioned meanings,
R¹⁴ represents C₁₋₃ alkyl, cyclopentyl, cyclohexyl or hydroxy-substituted C₂₋₃ alkyl, and
j represents 2, 3 or 4,
or, in case
A represents 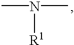
Q represents 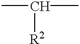
and
Z represents 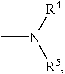
these
radicals together may represent a group of the formula 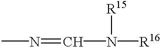
in which
R¹⁵ and R¹⁶ independently of one another represent C₁₋₃ alkyl or phenyl or
R¹⁵ and R¹⁶ together with the nitrogen atom, to which they are bonded, form a 5- or 6-membered heterocyclic group comprising at least one nitrogen atom or comprising at least one nitrogen atom and one oxygen atom,
with the proviso that
in case
Q represents a group of the formula 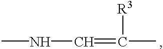
then
A represents 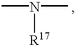
wherein
R¹⁷ represents a hydrogen atom or C₁₋₃ alkyl, and
Z represents cyano,
and in case
Q represents a group of the formula 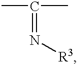
then
A represents —NH and
Z represents cyano
and in case
-(Q)ₖ-Z represents 2,3-dihydroxypropyl, then
A represents a sulphur atom or a group of the formula 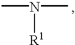
and in case
-(Q)ₖ-Z represents 2-hydroxyethyl and
A represents a group of the formula 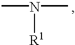
then
R¹ represents C₁₋₃ alkyl, cyclopentyl, cyclohexyl, phenyl or 2-hydroxyethyl
and in case
A represents a group of the formula 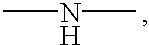
then
Q represents —CH₂— and
Z represents a group of the formula 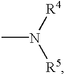
in which
R⁴ represents a hydrogen atom, benzyl or phenyl, the last two radicals being optionally substituted by 1 to 3 radicals selected from fluorine, chlorine, methyl and/or ethyl, and
R⁵ represents formyl,
and with the further proviso that
Z does not represent cyano or a group selected from 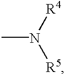
—OR⁶, —S(O)ₘ—R⁷ and 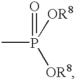
if
A is oxygen or sulphur and
k is o.

3. Isothiazolecarboxylic acid derivatives of the formula (I) according to claim 1,
A represents an oxygen atom, a sulphur atom or a group of the formula 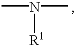
in which
R¹ represents a hydrogen atom, methyl, ethyl, n-propyl, iso-propyl, cyclopentyl, cyclohexyl, phenyl or 2-hydroxyethyl,
Q represents a group selected from 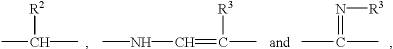
in which
R² represents a hydrogen atom, C₁₋₆ alkyl, trifluoromethyl, trichloromethyl, 2-phenylethyl or phenoxymethyl, which may be substituted by methoxycarbonyl, and
R³ represents phenyl, which may be substituted by 1 to 3 radicals selected from, fluoro, chloro, methyl, ethyl, trifluoromethyl, methoxy, trifluoromethoxy, phenoxy, benzyloxy, cyano and/or nitro, or may be mono-substituted by oxydimethylene,
k represents O or 1, and
Z represents a 5- or 6-membered heterocyclic ring comprising 1 to 4 nitrogen atoms, wherein the heterocyclic ring may be substituted by up to 3 substituents selected from fluorine, chlorine, methyl, ethyl, propyl, methoxy, trifluoromethyl, cyclopropyl, cyclopenthyl, 2-methyl-1-propenyl and/or phenyl, the latter radical being optionally substituted by 1 to 3 fluorine and/or chlorine atoms, and wherein the heterocyclic ring may also be mono- or disubstituted by oxo or spiro-bonded C₃₋₅ alicyclic groups, and wherein the heterocyclic ring may be condensed with a benzene or cyclohexene ring, or
Z represents a 5- or 6-membered heterocyclic ring comprising at least one nitrogen atom and one oxygen atom, or comprising at least one nitrogen atom and one sulphur atom, wherein each of the heterocyclic rings may be substituted by 1 to 3 substitutents selected from methyl and/or phenyl, and may also be substituted by 1 or 2 oxo groups, or
Z represents cyano or a group selected from 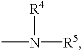
—O—R⁶, —S(O)ₘ—R⁷ and 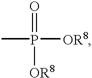
in which
R⁴ represents a hydrogen atom, methyl, ethyl, propyl, benzyl or phenyl, the last two radicals being optionally substituted by 1 to 3 radicals selected from fluorine, chlorine, methyl and/or ethyl, or
R⁴ represents tetrazol-5-yl-thiomethyl, which may be substituted by methyl,
R⁵ represents formyl, acetyl, pivaloyl, 3,4-dichloroisothiazol-5-yl-carbonyl, methylsulphonyl or phenylsulphonyl, or
R⁵ represents phenylcarbonyl, optionally substituted by 1 to 3 radicals selected from fluorine, chlorine and/or methyl,
R⁶ represents a hydrogen atom, methyl, ethyl, 2,2,3,3-tetrafluoropropyl, or represents benzyl or phenyl, each of which may be substituted by 1 to 3 radicals selected from fluorine and or chlorine, or represents benzoyl or phenylcarbamoyl, each of which may be substituted by trifluormethyl, or represents acetyl or propionyl,
R⁷ represents methyl, ethyl, phenyl or benzyl, the last two radicals being optionally substituted by 1 to 3 radicals selected from methyl, fluorine and/or chlorine, or
R⁷ represents tetrazol-5-yl or
R⁷ represents thiadiazol-2-yl optionally substituted by methyl or phenyl, or
R⁷ represents 2-thiazoline-2-yl, methylcarbonyl or benzoyl,
m represents O or 2, and
R⁸ represents methyl or ethyl,
or, in case
A represents a 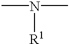
group, then
R¹, Q and Z together with the nitrogen atom of the 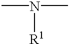
group may represent a 5- or 6-membered heterocyclic group comprising 1 or 2 nitrogen atoms and being optionally substituted by 1 to 3 radicals selected from methyl, ethyl, n-propyl, iso-propyl, tert-butyl, trifluoromethyl, hydroxy, oxo, hydroxymethyl and/or phenyl, which in turn may be substituted by 1 to 3 radicals selected from fluorine, chlorine and/or methyl, or
-(Q)ₖ-Z represents a group selected from 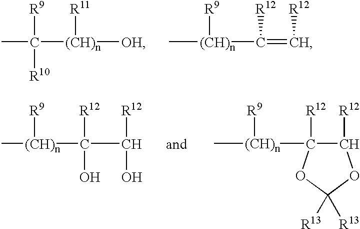
wherein
n represents 1 or 2,
R⁹ represents a hydrogen atom, methyl or ethyl,
R¹⁰ represents a hydrogen atom, hydroxymethyl or benzyl, which may be substituted by chlorine,
R¹¹ represents a hydrogen atom, methyl, ethyl, n-propyl, iso-propyl, tert-butyl or phenyl,
R¹² represents a hydrogen atom, methyl or phenyl, or two of the R¹² radicals, together with the atoms to which they are bonded, may form a 5- or 6-membered hydrocarbon ring, and
R¹³ represents a hydrogen atom, C₁₋₄ alkyl, cyclohexyl, 2-phenethyl, α-methylbenzyl, 2-cyclohexylethyl, ethoxymethyl, 2-ethoxyethyl or dimethoxymethyl, or the two R¹³ radicals, together with the carbon atom to which they are bonded, form a C₅₋₆ alicyclic ring which is optionally substituted by C₁₋₃ alkyl, or
-A-(Q)ₖ-Z represents —SH or a group of the formula 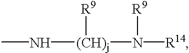
in which
R⁹ has the above-mentioned meanings,
R¹⁴ represents methyl, ethyl, cyclopentyl, cyclohexyl or hydroxyethyl, and
j represents 2 or 3,
or, in case
A represents 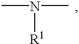
Q represents 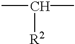
and
Z represents 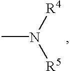
these
radicals together may represent a group of the formula 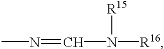
in which
R¹⁵ and R¹⁶ independently of one another represent methyl, ethyl or phenyl or
R¹⁵ and R¹⁶ together with the nitrogen atom, to which they are bonded, form a 5- or 6-membered heterocyclic group comprising at least one nitrogen atom or comprising at least one nitrogen atom and one oxygen atom,
with the proviso that
in case
Q represents a group of the formula 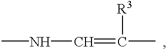
then
A represents —NH— or 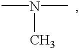
and
Z represents cyano,
and in case
Q represents a group of the formula 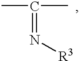
then
A represents —NH and
Z represents cyano
and in case
-(Q)ₖ-Z represents 2,3-dihydroxypropyl, then
A represents a sulphur atom or a group of the formula 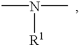
and in case
-(Q)ₖ-Z represents 2-hydroxyethyl and
A represents a group of the formula 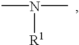
then
R¹ represents methyl, cyclopentyl, cyclohexyl, phenyl or 2-hydroxyethyl
and in case
A represents a group of the formula 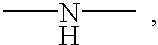
then
Q represents —CH₂— and
Z represents a group of the formula 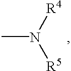
in which
R⁴ represents a hydrogen atom, benzyl or phenyl, the last two radicals being optionally substituted by 1 to 3 radicals selected from fluorine, chlorine, methyl and/or ethyl, and
R⁵ represents formyl,
and with the further proviso that
Z does not represent cyano or a group selected from 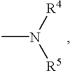
—OR⁶, —S(O)ₘ—R⁷ and 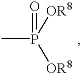
if
A is oxygen or sulphur and
k is o.

4. Process for the preparation of isothiazolecarboxylic acid derivatives of the formula (I), according to claim 1, characterized in that
a) the compound of the formula (I), in which
-A-(Q)ₖ-Z represents a group of the formula 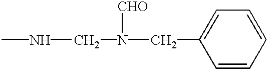
is prepared by reacting 3,4-dichloro-isothiazole-5-carboxamide of the formula 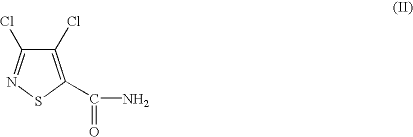
with the formylamine of the formula 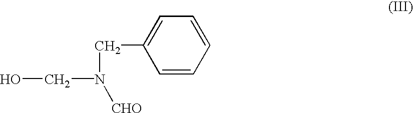
in the presence of an inert diluent and, if appropriate, in the presence of a catalyst, or
b) compounds of the formula (I), in which
-A-(Q)ₖ-Z represents a group of the formula 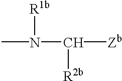
in which
R^{1b} represents a hydrogen atom or C₁₋₄ alkyl,
R^{2b} represents a hydrogen atom or C₁₋₄ haloalkyl and
Z^{b} represents a group selected from
—OR⁶, —SR⁷, —SO₂—R⁷ and 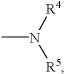
in which
R⁴, R⁵, R⁶ and R⁷ have the above-mentioned meanings,
are prepared by reacting isothiazole derivatives of the formula 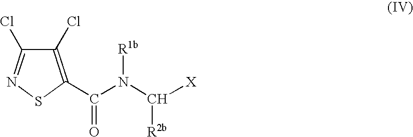
in which
R^{1b} and R^{2b} have the above-mentioned meanings and
X is chloro or bromo,
with compounds of the formula
M-Z^{b} (V)
in which
Z^{b} has the above-mentioned meanings and
M represents a hydrogen atom, lithium, sodium or potassium,
in the presence of an inert diluent and, if appropriate, in the presence of an acid binding agent and, if appropriate, in the presence of a phase-transfer catalyst, or
c) compounds of the formula (I), in which
-A-(Q)ₖ-Z represents a group of the formula 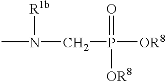
in which
R^{1b} and R⁸ have the above-mentioned meanings,
are prepared by reacting isothiazole derivatives of the formula 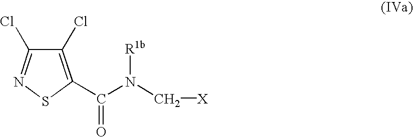
in which
R^{1b} and X have the above-mentioned meanings,
with phosphorous compounds of the formula
P(OR⁸)₃ (VI)
in which
R⁸ has the above-mentioned meanings,
in the presence of an inert diluent, or
d) compounds of the formula (I), in which
-A(Q)ₖ-Z represents a group of the formula
-A^{d}-CH₂-Z^{d},
in which
A^{d} represents 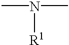
or a sulphur atom, wherein
R¹ has the above-mentioned meanings, and
Z^{d} represents a 5-7-membered heterocyclic ring comprising 1 to 4 nitrogen atoms, wherein the heterocyclic ring may be substituted by one or more substituents selected from halogen, C₁₋₄ alkyl, C₁₋₄ alkoxy, C₁₋₄ haloalkyl, C₃₋₆ cycloalkyl, C₂₋₄ alkenyl, phenyl or
Z^{d} represents a 5-7-membered heterocyclic ring comprising at least one nitrogen atom and one oxygen atom, or comprising at least one nitrogen atom and one sulphur atom, wherein each of the heterocyclic rings may be substituted by one or more substituents selected from C₁₋₄ alkyl, phenyl and/or oxo or
Z^{d} represents a group selected from 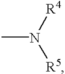
—OR and —SR⁷
in which
R⁴, R⁵, R⁶ and R⁷ have the above-mentioned meanings,
are prepared by reacting isothiazole derivatives of the formula 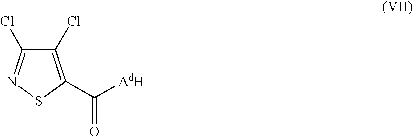
in which
A^{d} has the above-mentioned meaning,
with chloromethyl compounds of the formula
Cl—CH₂-Z^{d} (VIII)
in which
Z^{d} has the above-mentioned meanings,
in the presence of an inert diluent and, if appropriate, in the presence of an acid binding agent, or
c) compounds of the formula (I), in which
-A-(Q)ₖ-Z represents a group of the formula 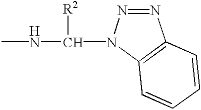
in which
R² has the above-mentioned meanings,
are prepared by reacting 3,4-dichloro-isothiazole-5-carboxamide of the formula 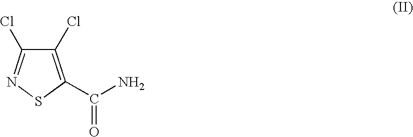
with formyl compounds of the formula
R²—CHO (IX)
in which
R² has the above-mentioned meanings,
and with 1H-benzotriazole of the formula 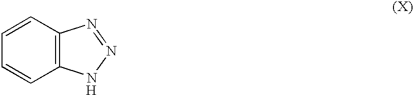
in the presence of an inert diluent and, if appropriate, in the presence of a catalyst, or
f) compounds of the formula (I), in which
-A-(Q)ₖ-Z represents —SH or a group selected from 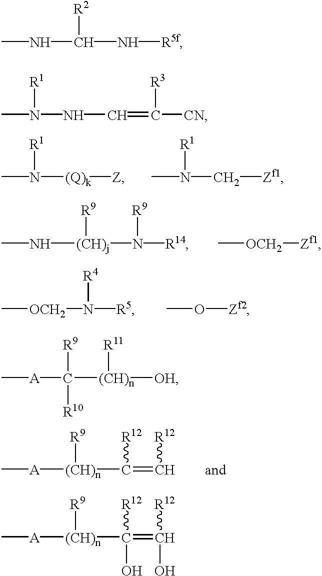
in which
A, Q, Z, j, k, n, R¹, R², R³, R⁴, R⁵, R⁹, R¹⁰, R¹¹, R¹² and R¹⁴ have the above-mentioned meanings,
Z^{f1} represents a 5-7-membered heterocyclic ring comprising 1 to 4 nitrogen atoms, wherein the heterocyclic ring may be substituted by one or more substituents selected from halogen, C₁₋₄ alkyl, C₁₋₄ alkoxy, C₁₋₄ haloalkyl, C₃₋₆ cycloalkyl, C₂₋₄ alkenyl, phenyl, halophenyl, oxo and/or spiro-bonded C₃₋₆ alicyclic groups, and wherein the heterocyclic ring may be condensed with a benzene or cyclohexene ring, or
Z^{f1} represents a 5-7-membered heterocyclic ring comprising at least one nitrogen atom and one oxygen, or comprising at least one nitrogen atom and one sulphur atom, wherein each of the heterocyclic rings may be substituted by one or more substituents selected from C₁₋₄ alkyl, phenyl and/or oxo,
Z^{f2} represents a 5-membered heterocyclic group comprising 1 or 2 nitrogen atoms, which heterocycle may be substituted by C₁₋₄ alkyl and/or oxo, and
R^{5f} represents formyl, C₁₋₄ alkylcarbonyl or phenylcarbonyl, this latter radical being optionally substituted by 1 to 3 radicals selected from halogen and C₁₋₄ alkyl,
are prepared by reacting 3,4-dichloro-isothiazole-5-carbonyl chloride of the formula 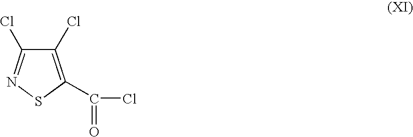
with compounds of the formula
M-Y, (XII)
in which
M has the above-mentioned meanings and
Y¹ represents —SH or a group selected from 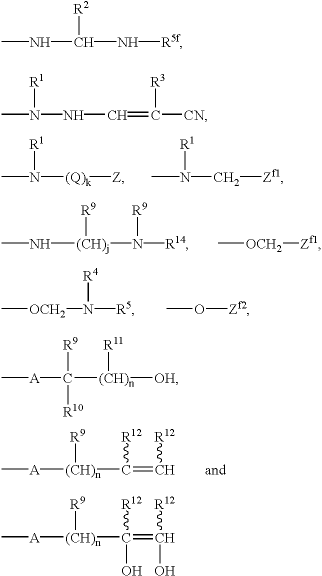
in which
A, Q, Z, j, k, n, R¹, R², R³, R⁴, R⁵, R⁹, R¹⁰, R¹¹, R¹², R¹⁴, Z^{f1}, Z^{f2}, and R^{5f} have the above-mentioned meanings,
in the presence of an inert diluent and, if appropriate, in the presence of an acid binding agent, or
g) compounds of the formula (I), in which
-A-(Q)ₖ-Z represents a group selected from 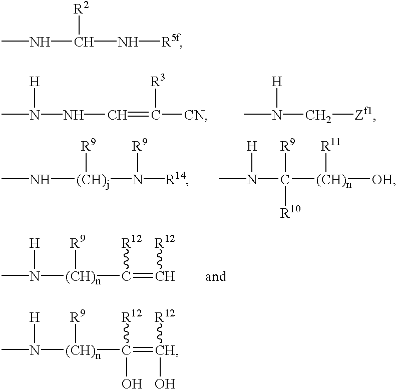
in which
Z^{f1}, j, n, R², R³, R⁹, R¹⁰, R¹¹, R¹², R¹⁴ and R^{5f} have the above-mentioned meanings,
are prepared by reacting 3,4-dichloro-isothiazole-5-carboxylic acid esters of the formula 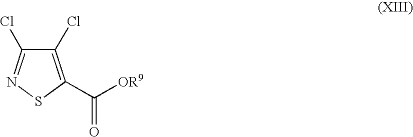
in which
R^{g} represents C₁₋₄ alkyl
with compounds of the formula
H—Y² (XIV)
in which
Y² represents a group selected from 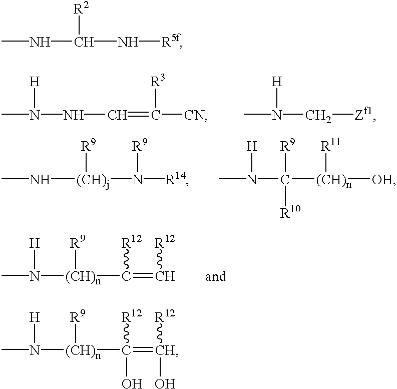
in which
Z^{f1}, j, n, R², R³, R⁹, R¹⁰, R¹¹, R¹², R¹⁴, and R^{5f} have the above-mentioned meanings,
in the presence of an inert diluent and, if appropriate, in the presence of an acid binding agent, or
h) compounds of the formula (I), in which
-A-(Q)ₖ-Z represents a group of the formula 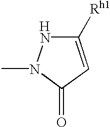
in which
R^{h1} represents phenyl optionally substituted by halogen and/or C₁₋₄ alkyl,
are prepared by reacting 3,4-dichloro-isothiazole-5-carbohydrazide of the formula 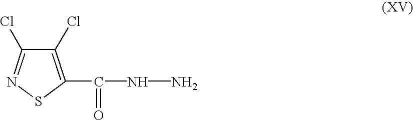
with compounds of the formula 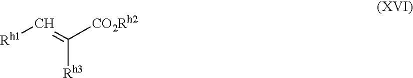
in which
R^{h1} has the above-mentioned meanings,
R^{h2} represents C₁₋₄ alkyl and
R^{h3} represents cyano or —COOR^{h2},
in the presence of an inert diluent and, if appropriate, in the presence of an acid binding agent and, if appropriate, in the presence of a catalyst, or
i) compounds of the formula (I), in which
-A-(Q)ₖ-Z represents a group of the formula 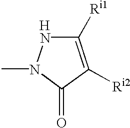
in which
Rⁱ¹ represents a hydrogen atom or C₁₋₄ alkyl or represents phenyl optionally substituted by halogen and/or C₁₋₄ alkyl and
Rⁱ² represents a hydrogen atom or C₁₋₄ alkyl,
can be prepared by reacting 3,4-dichloro-isothiazole-5-carbohydrazide of the formula 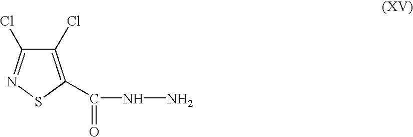
with compounds of the formula 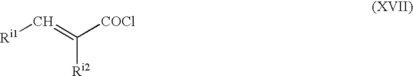
in which
Rⁱ¹ and Rⁱ² have the above-mentioned meanings,
in the presence of an inert diluent and, it appropriate, in the presence of an acid binding agent, or
j) compounds of the formula (I), in which
-A-(Q)ₖ-Z represents a group of the formula 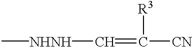
in which
R³ has the above-mentioned meanings,
can be prepared by reacting 3,4-dichloro-isothiazole-5-carbohydrazide of the formula 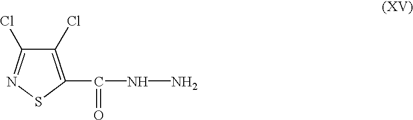
with compounds of the formula 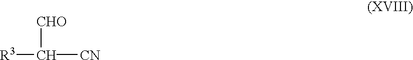
in which
R³ has the above-mentioned meanings,
in the presence of an inert diluent and, if appropriate, in the presence of a catalyst, or
k) compounds of the formula (I), in which
-A-(Q)ₖ-Z represents a group of the formula 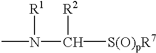
in which
R¹, R² and R⁷ have the above-mentioned meanings and
p denotes 1 or 2,
can be prepared by reacting isothiazolecarboxylic acid derivatives of the formula 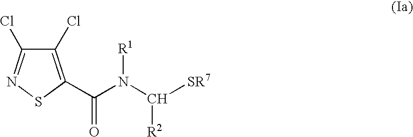
in which
R¹, R² and R⁷ have the above-mentioned meanings,
with oxidizing agents, which are suitable for providing oxygen, in the presence of an inert diluent, or
l) compounds of the formula (I), in which
-A-(Q)ₖ-Z represents a group of the formula 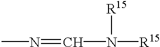
in which
R¹⁵ has the above-mentioned meanings,
are prepared by reacting 3,4-dichloro-isothiazole-5-carboxamide of the formula 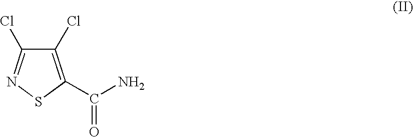
with compounds of the formula 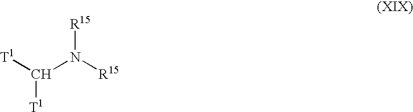
in which
R¹⁵ has the above-mentioned meanings and
T¹ represents C₁₋₄ alkoxy,
in the presence of an inert diluent and, if appropriate, in the presence of a catalyst, or
m) compounds of the formula (I), in which
-(Q)ₖ-Z represents a group of the formula 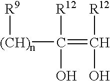
in which
R⁹, R¹² and n have the above-mentioned meanings,
are prepared by reacting isothiazolecarboxylic acid derivatives of the formula 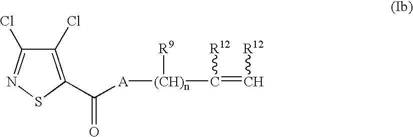
in which
A, R⁹, R¹² and n have the above-mentioned meanings,
with oxidizing agents, which are suitable for providing oxygen, in the presence of water and, if appropriate, in the presence of an inert organic diluent, or
n) compounds of the formula (I), in which
-(Q)ₖ-Z represents a group of the formula 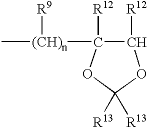
in which
R⁹, R¹², R¹³ and n have the above-mentioned meanings,
are prepared by reacting isothiazolecarboxylic acid derivatives of the formula 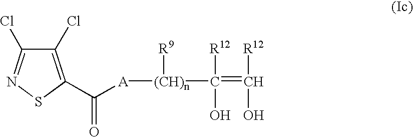
in which
A, n, R⁹ and R¹² have the above-mentioned meanings,
with carbonyl derivatives of formula 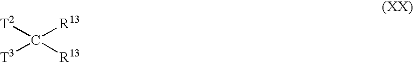
in which
R¹³ has the above-mentioned meanings and
T² represents C₁₋₄ alkoxy or the two T²-radicals together represent and oxo group,
in the presence of an inert diluent and, if appropriate, in the presence of an acid catalyst, or
o) compounds of the formula (I), in which
-A-(Q)ₖ-Z represents a group of the formula 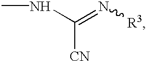
in which
R³ has the above-mentioned meanings,
are prepared by reacting 3,4-dichloro-isothiazole-5-carboxamide of the formula 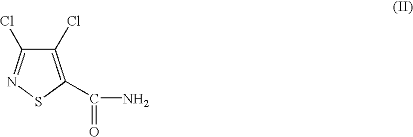
with cyano compounds of the formula 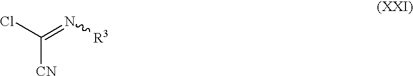
in which
R³ has the above-mentioned meanings,
in the presence of an inert diluent and, if appropriate, in the presence of an acid binding agent, or
p) compounds of the formula (I), in which
-A-(Q)ₖ-Z represents a group of the formula 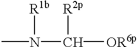
in which
R^{1b} has the above-mentioned meanings,
R^{2p} represents a hydrogen atom or C₁₋₄ haloalkyl and
R^{6p} represents a hydrogen atom or C₁₋₄ alkyl,
can be prepared by reacting 3,4-dichloro-isothiazole derivatives of the formula 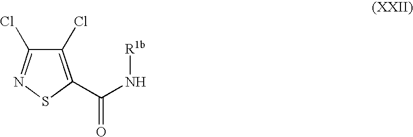
in which
R^{1b} has the above-mentioned meanings,
with compounds of the formula 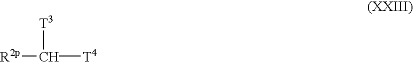
in which
R^{2p} has the above-mentioned meanings,
T³ represents hydroxy and
T⁴ represents C₁₋₄ alkoxy or
T³ and T⁴ together represent and oxo group,
in the presence of an inert diluent and, if appropriate, in the presence of an acid binding agent, or
q) compounds of the formula (I), in which
-A-(Q)ₖ-Z represents a group of the formula 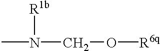
in which
R^{1b} has the above-mentioned meanings and
R^{6q} represents C₁₋₄ alkyl-carbonyl or benzoyl, which may be substituted by C₁₋₄ haloalkyl
are prepared by reacting 3,4-dichloro-isothiazole derivatives of the formula 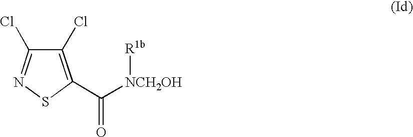
in which
R^{1b} has the above-mentioned meanings,
with chloro-substituted compounds of the formula
Cl—R^{6q} (XXIV)
in which
R^{6q} has the above-mentioned meanings,
in the presence of an inert diluent and, if appropriate, in the presence of an acid binding agent, or
r) compounds of the formula (I), in which
-A-(Q)ₖ-Z represents a group of the formula 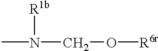
in which
R^{1b} has the above-mentioned meanings and
R^{6r} represents phenylcarbamoyl or C₁₋₄ haloalkyl-substituted phenylcarbamoyl
are prepared by reacting 3,4-dichloro-isothiazole derivatives of the formula 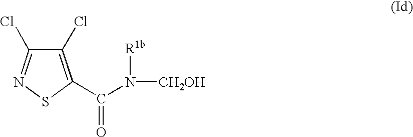
in which
R^{1b} has the above-mentioned meanings,
with isocyanates of the formula
O═C=N—R^{r} (XXV)
in which
R^{r} represents phenyl or C₁₋₄ haloalkyl-substituted phenyl,
in the presence of an inert diluent and, if appropriate, in the presence of an acid binding agent and, if appropriate, in the presence of a base catalyst.

5. Microbicidal compositions, characterized in that they contain at least one isothiazolecarboxylic acid derivative of the formula (I) according to claim 1 plus extenders and/or surface active agents.

6. Process for combating undesired microorganisms, characterized in that isothiazolecarboxylic acid derivatives of the formula (I) according to claim 1 are applied to the microorganisms and/or to their habitat.

7. Use of isothiazolecarboxylic acid derivatives of the formula (I) according to claim 1 for combating undesired microorganisms.

8. Process for the preparation of microbicidal compositions, characterized in that isothiazolecarboxylic acid derivatives of the formula (I) according to claim 1 are mixed with extenders and/or surface-active agents.

9. Isothiazolecarboxylic acid derivatives of the formula 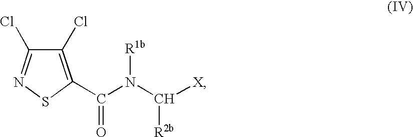
wherein
R^{1b} represents a hydrogen atom or C₁₋₄ alkyl,
R^{1b} represents a hydrogen atom or C₁₋₄ haloalkyl and
X represents chloro or bromo.
